SEQUENCE LISTING (40 sequences)

SEQ ID NO: 1 - DNA, 20 bp, Artificial Sequence
  Synthetic Primer
attgggatcatcttgctggt

SEQ ID NO: 2 - DNA, 19 bp, Artificial Sequence
  Synthetic Primer
cctgctgttcacagttgcc

SEQ ID NO: 3 - DNA, 20 bp, Artificial Sequence
  Synthetic Primer
gtggaatcttccggctgtag

SEQ ID NO: 4 - DNA, 20 bp, Artificial Sequence
  Synthetic Primer
accatgacactctgcaacca

SEQ ID NO: 5 - DNA, 20 bp, Artificial Sequence
  Synthetic Primer
gaaacagcaggaagtgggag

SEQ ID NO: 6 - DNA, 20 bp, Artificial Sequence
  Synthetic Primer
catgaagctctgcgtgtctg

SEQ ID NO: 7 - DNA, 20 bp, Artificial Sequence
  Synthetic Primer
ccacttcttctctgggttgg

SEQ ID NO: 8 - DNA, 20 bp, Artificial Sequence
  Synthetic Primer
gtgcccacgtcaaggagtat

SEQ ID NO: 9 - DNA, 20 bp, Artificial Sequence
  Synthetic Primer
taggcaggtttgatctccgt

SEQ ID NO: 10 - DNA, 21 bp, Artificial Sequence
  Synthetic Primer
cgatccactacaaatccctca

SEQ ID NO: 11 - DNA, 20 bp, Artificial Sequence
  Synthetic Primer
cctatggccctcattctcac

SEQ ID NO: 12 - DNA, 20 bp, Artificial Sequence
  Synthetic Primer
ctcatcctgctgggtctgag

SEQ ID NO: 13 - DNA, 20 bp, Artificial Sequence
  Synthetic Primer
cgcccctgtttgaacataag

SEQ ID NO: 14 - DNA, 21 bp, Artificial Sequence
  Synthetic Primer
ctgctgagatgaacaggaagg

SEQ ID NO: 15 - DNA, 20 bp, Artificial Sequence
  Synthetic Primer
cccagtgctggagaaattgt

SEQ ID NO: 16 - DNA, 20 bp, Artificial Sequence
  Synthetic Primer
ccctatggagatgacggaga

SEQ ID NO: 17 - DNA, 20 bp, Artificial Sequence
  Synthetic primer
tgagctcattgaatgcttgg

SEQ ID NO: 18 - DNA, 20 bp, Artificial Sequence
  Synthetic Primer
acagcaaggcgaaaaaggat

SEQ ID NO: 19 - DNA, 20 bp, Artificial Sequence
  Synthetic Primer
agggtctgggccatagaact

SEQ ID NO: 20 - DNA, 21 bp, Artificial Sequence
  Synthetic Primer
ccaccacgctcttctgtctac

SEQ ID NO: 21 - DNA, 20 bp, Artificial Sequence
  Synthetic Primer
ccagaagaaaatgaggtcgg

SEQ ID NO: 22 - DNA, 19 bp, Artificial Sequence
  Synthetic Primer
agcgctcaaggagaagacc

SEQ ID NO: 23 - DNA, 20 bp, Artificial Sequence
  Synthetic Primer
ggtcaaaggtttggaagcag

SEQ ID NO: 24 - DNA, 20 bp, Artificial Sequence
  Synthetic Primer
tgtgaaatgccaccttttga

SEQ ID NO: 25 - DNA, 23 bp, Artificial Sequence
  Synthetic Primer
accagaggaaattttcaataggc

SEQ ID NO: 26 - DNA, 20 bp, Artificial Sequence
  Synthetic Primer
tgatgcacttgcagaaaaca

SEQ ID NO: 27 - DNA, 20 bp, Artificial Sequence
  Synthetic Primer
gccccgtggctcagtgaagg

SEQ ID NO: 28 - DNA, 20 bp, Artificial Sequence
  Synthetic Primer
ctgactagcggcctgggaca

SEQ ID NO: 29 - DNA, 20 bp, Artificial Sequence
  Synthetic Primer
acttctgcgcgacggttggg

SEQ ID NO: 30 - DNA, 21 bp, Artificial Sequence
  Synthetic Primer
tgggggaacgggcattgcttc

SEQ ID NO: 31 - DNA, 20 bp, Artificial Sequence
  Synthetic Primer
cgtgaacatgttgttgaggc

SEQ ID NO: 32 - DNA, 20 bp, Artificial Sequence
  Synthetic Primer
cgaatctgcaccgtagttga

SEQ ID NO: 33 - DNA, 20 bp, Artificial Sequence
  Synthetic Primer
cggtgtcagagtctagggga

SEQ ID NO: 34 - DNA, 20 bp, Artificial Sequence
  Synthetic Primer
cgaagtcaaagttccaccgt

SEQ ID NO: 35 - DNA, 20 bp, Artificial Sequence
  Synthetic Primer
gccaggatgtcagcgggagc

SEQ ID NO: 36 - DNA, 19 bp, Artificial Sequence
  Synthetic Primer
aaggccgggcttcttgggc

SEQ ID NO: 37 - DNA, 20 bp, Artificial Sequence
  Synthetic Primer
gacccgactccggacccgat

SEQ ID NO: 38 - DNA, 20 bp, Artificial Sequence
  Synthetic Primer
agtcgttcgcattggccgca

SEQ ID NO: 39 - DNA, 18 bp, Artificial Sequence
  Synthetic Primer
aacgaccccttcattgac

SEQ ID NO: 40 - DNA, 19 bp, Artificial Sequence
  Synthetic Primer
tccacgacatactcagcac

(19) United States
(12) Patent Application Publication
(10) Pub. No.: US 2015/0024008 A1
(43) Pub. Date: Jan. 22, 2015

(54) Methods and Compositions Involving Induced Senescent Cells for Cancer Treatment

(71) Applicant: THE UNIVERSITY OF CHICAGO, Chicago, IL (US)

(72) Inventors: Stephen Kron, Chicago, IL (US); Ralph R. Weichselbaum, Chicago, IL (US); Elena Efimova, Chicago, IL (US); Yuru Meng, Chicago, IL (US); Amy Flor, Chicago, IL (US)

(21) Appl. No.: 14/359,745

(22) PCT Filed: Nov. 21, 2012
(86) PCT No.: PCT/US2012/066369

§ 371 Date: May 21, 2014

Related U.S. Application Data
(60) Provisional application No. 61/562,117, filed on Nov. 21, 2011.

(51) Int. Cl. A61K 35/00

(52) U.S. Cl. CPC A61K 35/13; A61K 2035/122
USPC 424/277.1; 435/375; 435/173.1

(57) ABSTRACT

Disclosed are cancer vaccines comprising senescent cells and methods of using and preparing the vaccines.

The application claims priority to U.S. Provisional Patent Application 61/562,117 filed on Nov. 21, 2011, which is hereby incorporated by reference.

The invention was made with government support under Grants No. CA138365, CA164492 and GM60443 awarded by the National Institutes of Health. The government has certain rights in the invention.

### BACKGROUND OF THE INVENTION

1. Field of the Invention

The present invention relates generally to the field of medicine. More particularly, it concerns compositions and methods for evoking an immune response to cancer cells by introducing into a subject induced senescent cells.

2. Description of Related Art

Although patients with advanced cancer may obtain significant benefit from radiotherapy, failure frequently occurs due to local recurrence or distant metastasis. Ongoing advances in radiation delivery and chemical radiosensitizers have improved local control but approaches to preventing and treating metastasis remain elusive. Therefore, the potential for radiation to reliably induce a sustained anti-tumor immune response as a route to preventing relapse or metastasis has yet to be realized.

### SUMMARY OF THE INVENTION

Methods and compositions are provided in embodiments described herein. Methods and compositions concern induced senescent cells for use in treating cancer in a patient. In other embodiments, it concerns using antigen presenting cells exposed to induced senescent cells in treating cancer.

In some embodiments, there are methods for preparing or manufacturing a pharmaceutical composition of cancer cells comprising: a) exposing cancer cells removed from a patient to an effective amount of radiation and/or at least one senescence inducing agent to induce senescence; b) purifying or enriching for induced senescent cells; and c) preparing a pharmaceutical composition of induced senescent cells.

In further embodiments, there are methods for treating a cancer patient comprising administering to the patient a pharmaceutical composition comprising induced senescent cells from the patient, wherein the pharmaceutical composition was prepared according to the methods disclosed herein.

Additional embodiments concern methods for treating a cancer patient comprising administering to the patient induced senescent cells, wherein the induced senescent cells are prepared from cancer cells obtained from the patient.

Other embodiments include methods for treating a cancer patient comprising: a) obtaining or retrieving cancer cells from the patient; b) exposing the cancer cells to an effective amount of radiation and at least one senescence inducing agent to induce senescence; c) purifying the induced senescent cells; and, d) administering the induced senescent cells to the patient.

Other embodiments involve methods for preparing a pharmaceutical composition of senescent cells comprising: a) exposing cancer cells removed from a patient to an effective amount of radiation and at least one senescence inducing agent to induce senescence; b) enriching for induced senescent cells using flow cytometry; and, c) preparing a pharmaceutical composition of induced senescent cells.

More embodiments provide for pharmaceutical compositions comprising induced senescent cells, wherein the senescent tumor cells have a least one of the following characteristics compared to cancer cells not exposed to radiation and/or a senescence inducing agent reduced cell proliferation rate; increased β-galactosidase activity; increased size; reduced expression of p16INK4a; increased expression of p21Cip1p; increased lyosomal mass; nuclear loci of persistent DNA damage response; and, altered expression or secretion of amphiregulin, growth-related oncogene (GRO) γ, interleukin 6 (IL-6), IL-8, VEGF, and/or matrix metalloproteinase.

Pharmaceutical compositions may be made, prepared, or manufactured using any method provided herein.

In further embodiments, there are methods for preparing a pharmaceutical composition comprising antigen presenting cells comprising exposing antigen presenting cells to induced senescent cells that were previously induced from cancer cells; and, preparing a pharmaceutical composition comprising exposed antigen presenting cells.

Furthermore, there are methods for treating a cancer patient comprising administering to the patient induced senescent cells, wherein the induced senescent cells are prepared from cancer cells previously obtained from the patient.

In certain embodiments, there are pharmaceutical compositions comprising antigen presenting cells comprising an antigen from an induced senescent cell. It is contemplated that the antigen presented by the antigen presenting cell is from the induced senescent cell that is derived a cancer cell of a patient. In particular embodiments, the antigen presenting cells are autologous. Embodiments concern antigen presenting cells, or precursors thereof, from the same patient who is the source of the cancer cells that are induced to senesce or become senescent.

A cancer patient may be a patient who has cancer or symptoms of cancer, a patient who previously had cancer, a patient at risk for cancer recurrence, a patient with or at risk for metastatic cancer, or a patient previously treated for cancer. It is further contemplated that in some embodiments, the cancer includes cells determined to be a pre-cancer, hyperplasia, or dysplasia. In some embodiments, the cancer is a semi-solid or solid tumor. In other embodiments, the cancer may be or include cells from inside or from the cell wall of a cyst or other lesion.

The term “individual,” “subject,” or “patient” refers to humans, but embodiments may be extended to other animals including, e.g., other primates, rodents, canines, felines, equines, ovines, porcines, and other mammals.

Embodiments may involve about, at least about, or at most about 10², 10³, 10⁴, 10⁵, 10⁶, 10⁷, 10⁸, 10⁹, 10¹⁰, 10¹¹, or 10¹² (or any range derivable therein) cancer cells that are exposed to an effective amount of radiation and/or at least one senescence inducing agent. In additional embodiments, there are about, at least about, or at most about 10², 10³, 10⁴, 10⁵, 10⁶, 10⁷ 10⁸, 10⁹, 10¹⁰, 10¹¹, or 10¹² (or any range derivable therein) induced senescent cells in methods and compositions.

In some embodiments, cancer cells are exposed to radiation. The cancer cells are exposed to an effective amount of radiation alone or in combination with at least one senescence inducing agent. In some embodiments, the cancer cells are exposed to about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Gy of radiation (and any range derivable therein). It is contemplated that cells may be exposed to radiation more than once. They may be exposed, 1, 2, 3, 4, 5 or more time (or any range derivable therein). The radiation is gamma radiation in some embodiments. In certain embodiments, cancer cells are exposed to radiation and at least one senescing inducing agent. In some cases, cancer cells are exposed to radiation and 1, 2, 3, 4, 5, or more senescence inducing agents.

In some embodiments, the cancer cells are exposed to, contacted with, mixed with, or incubated with an effective amount of at least one senescence inducing agent. A senescence inducing agent refers to a compound or chemical that induces cell senescence. Such senescence inducing agents include those compounds listed in Table 3. It is contemplated that cells may be exposed to a senescence inducing agent more than once. They may be exposed, 1, 2, 3, 4, 5 or more time (or any range derivable therein). In some cases, the cancer cells are exposed to different senescence inducing agents, which may or may not be at the same time.

For a cancer cell, senescence may be qualified by having at least or at most 1, 2, 3, 4, 5, 6, 7, 8, 9, 10, 11, 12 or all of the following characteristics: reduced cell proliferation rate; increased β-galactosidase activity; increased size; reduced expression of p16INK4a; increased expression of p21Cip1p; increased lyosomal mass; nuclear loci of persistent DNA damage response; and, altered expression or secretion of amphiregulin, growth-related oncogene (GRO) γ, interleukin 6 (IL-6), IL-8, VEGF, and/or matrix metalloproteinase. Additional characteristics include, but are not limited to, at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more (or any range derivable therein) of the following: increase in expression or activity of cell cycle inhibitory proteins of p16, p38, p21, or p53; increase in disruption to downstream cell signaling cascades; persistent or increased DNA damage response (DDR); increased reactive oxygen species (ROS); appearance of heterochromatin condensation and rearrangement; altered expression of one or more Senescence-Associated Secretory Phenotype (SASP) cytokine; low energy metabolism; change in morphology (larger, flatter, highly granular); growth arrest in G0/G1; overexpression of a number of genes including, but not limited to, SM22, MMP1, and/or IFN-γ; deletion of mitochondrial DNA; telomere shortening; increase in lysosomal β-Gal activity; and, nuclear accumulation of G-Action and depolymerization of F-actin. In particular embodiments, an increase in expression and/or activity of 1, 2, 3, 4, 5, 6, 7, 8, 9, 10 or more (or any range derivable therein) of the following may be measured or evaluated or be the basis for determining senescence: IL-6, IL-7, IL-1a, IL-1b, IL-13, IL-15, IL-8, GRO-a,GRO-b, GRO-g, MCP-2, MCP-4, MIP-1a, MIP-3a, HCC-4, Eotaxin-3, GM-CSF, MIF, Amphiregulin, Epiregulin, Heregulin, EGF, bFGF, HGF, KGF (FGF7), VEGF, Angiogenin, SCF, SDF-1, PIGF, IGFBP-2, IGFBP-3, IGFBP-4, IGFBP-6, IGFBP-7, MMP-1, MMP-3, MMP-10, MMP-12, MMP-13, MMP-14, TIMP-2, PAI-1, PAI-2, tPA, uPA, Cathepsin B, ICAM-1, ICAM-3, OPG, sTNFRI, TRAIL-R3, Fas, sTNFRII, Fas, uPAR, SGP130, EGF-R, PGE2, Nitric oxide, or Fibronectin. In further embodiments, a decrease in expression and/or activity of TIMP-1 may be measured or evaluated or be the basis for determining senescence. In some embodiments, a change in expression and/or activity of 1, 2, or 3 of the following may be measured or evaluated or be the basis for determining senescence: Reactive oxygen species, Collagen, or Laminin. In some embodiments, one or more of the following are not used as a marker for senescence: TECK, ENA-78, I-309, I-TAC, Eotaxin, G-CSF, IFN-gamma, BLC, and/or NGF.

An quantitative or qualitative difference may be evaluated based on a comparison with a reference or standard, such as a cancer cell not exposed to the same conditions as far as radiation and/or senescence inducing agent(s). Alternatively, the reference or standard may be a normal or a noncancerous cell. A difference may be an increase or decrease of about, at least about, or at most about 5, 10, 15, 20, 25, 30, 35, 40, 45, 50, 55, 60, 65, 70, 75, 80, 85, 90, 95 or 100% (and any range derivable therein) and/or of about, at least about, or at most about 1.5×, 2×, 2.5×, 3×, 3.5×, 4×, 4.5× 5×, 10×, 20×, 30×, 40×, 50×, 60×, 70×, 80×, 90×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 410×, 420×, 430×, 440×, 450×, 460×, 470×, 480×, 490×, 500×, 600×, 700×, 800×, 900×, 1000×, 1100×, 1200×, 1300×, 1400×, 1500×, 1600×, 1700×, 1800×, 1900×, 2000×, 3000×, 4000×, 5000×, 6000×, 7000×, 8000×, 9000×, 10,000× or more, or any range derivable therein.

In some embodiments, 1, 2, 3, 4, 5, 6, 7, 8, 9, or 10 senescence inducing agents (or any range derivable therein) are used with or without radiation. In certain embodiments, one or more senescence inducing agents is in pharmaceutically acceptable formulation. Particular embodiments involve a senescence inducing agent that is a tumor suppressor inducer, mitotic inhibitor, nucleic acid damaging agent, antitumor antibiotic, topoisomerase inhibitor, hormone inhibitor, growth factor inhibitor, or PARP inhibitor. In further embodiments the senescence inducing agent is an inhibitor of histone acetyltransferases (HATs), a histone deacetylase (HDAC), DNA methyltransferase (DNMT), demethylase, histone ubiquitylase, a deubiquitination enzyme, histone chaperone, histone exchange complex, chromatin remodeler, inhibitor of the NAD+ salvage pathway, inhibitor of nicotinamide phosphoribosyltransferase (NAMPT), low glucose cell growth conditions (glucose limitation), a compound targeting glycolytic metabolism, a glucose transporter inhibitor, hexokinase 2, phosphofructokinase 2 inhibitor, phosphofructo-2-kinase/fructose-2,6-bisphosphatase 3 inhibitor, pyruvate kinase (PK) inhibitor, pyruvate kinase M2 inhibitor, lactate dehydrogenase (LDH) inhibitor, LDH5 lactate dehydrogenase 5 inhibitor, carbonic anhydrase-9 inhibitor, activator of oxidative phosphorylation and pyruvate dehydrogenase (PDH) complex activator, pyruvate dehydrogenase kinase inhibitor, membrane-bound V-ATPase inhibitor, monocarboxylate transporter 1 inhibitor, Adenosine Monophosphate-Activated Protein Kinase activator, or a hypoxia-inducible factor-1 inhibitor.

In specific embodiments, a senescence inducing agent is Trazodone, Ketotifen, Cephalexin, Nisoldipine, CGS15943, Clotrimazole, 5-Nonyltryptamine, Doxepin, Pergolide, Paroxetine, Resveratrol, Quercetin, Honokiol, 7-nitroindazole, Megestrol, Fluvoxamine, Etoposide, Veliparib, Rucaparib, Olaparib, Camptothecin, or Terbinafine.

The term “effective amount” refers an amount that achieves the stated goal. In the case of inducing senescence, an effective amount refers to an amount that induces senescence in cells. In certain embodiments, senescence is induced in at least about 10, 15, 20, 25, 30, 35, 40, 45, 50, 55, 60, 65, 70, 75, 80, 85, 90, 95% or more of a cell population (or any range derivable therein). It is contemplated that in some embodiments, cancer cells are exposed to a senescence inducing agent and/or radiation for about, at least about or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, and/or 1, 2, 3, 4, 5 weeks (and any range derivable therein).

In some embodiments, induced senescent cells are enriched or purified by sorting senescent cells from non-senescent cells. A cell population may be enriched or purified such that 50, 55, 60, 65, 70, 75, 80, 85, 90, 95% or more cells in the cell population (and any range derivable therein) are the type being selected for, such as induced senescent cells. In some cases, the cell population is enriched 2×, 3×, 4×, 5×, 6×, 7×, 8×, 9×, 10× or more (or any range derivable therein) for induced senescent cells. In some embodiments, induced senescent cells are enriched or purified using β-galactosidase expression. In further embodiments, sorting comprises using flow cytometry. In specific embodiments, purifying or enriching for induced senescent cells comprises incubating a β-galactosidase substrate with cancer cells exposed to radiation and/or at least one senescence inducing agent and selecting for β-galactosidase activity. In some cases, β-galactosidase activity is detectable upon cleavage of the β-galactosidase substrate by β-galactosidase. In some embodiments, β-galactosidase activity is detectable after cleavage. A label or other detectable moiety may be employed for evaluating whether a cell is been induced into senescence or for sorting, separating, or selecting induced senescent cells and non-senescent cells. In particular embodiments, β-galactosidase activity is detectable by fluorescence. In some cases, a substrate of β-galactosidase is employed and the enzymatic product is detectable, such as by fluorescence.

In certain embodiments, there is also a step of obtaining or retrieving the cancer cells from the patient. The cancer cells may be obtained by surgical resection, by vacuum, by fine needle aspirate, by extracting cystic fluid, by a tissue scrape, or by other means for removal.

In some embodiments, a cytological evaluation may be done on cells. For instance, a cytological evaluation may be done identify and/or select cancer or tumor cells from a patient. Morphology of cells retrieved from the patient may be evaluated to identify cancer or tumor cells. In further embodiments, identifying induced senescent cells may involve performing a cytological evaluation. The size and/or morphology of cells may be evaluated. Moreover, enzymatic activity may be evaluated using labeled substrates, detectable moieties attached to substrates, or enzymatic products that are detectable.

Further embodiments involve culturing cancer cells obtained from the patient before exposing the cancer cells to radiation and/or a senescence inducing agent. The cancer cells may be passaged at least or at most 1, 2, 3, 4, 5, 6, 7, 8, 9, 10 or more times (or any range derivable therein).

In some therapeutic regimens, methods involve administering to the patient induced senescent cells. In some cases, a batch of such cells is administered to the patient at least or at most 1, 2, 3, 4, 5 or more times (or any range derivable therein). In further embodiments, methods also involve administering to the patient radiation and/or chemotherapy. In some embodiments, a patient is administered an immunotherapy as part of a therapeutic regimen. In specific embodiments, the patient is administered radiation.

As discussed above, senescent cells may have at least or at most 1, 2, 3, 4, 5, 6, 7, 8, 9, 10, 11, 12 or all 13 (or any range derivable therein) of the following characteristics: reduced cell proliferation rate; increased β-galactosidase activity; increased size; reduced expression of p16INK4a; increased expression of p21Cip1p; increased lyosomal mass; nuclear loci of persistent DNA damage response; and, altered expression or secretion of amphiregulin, growth-related oncogene (GRO) γ, interleukin 6 (IL-6), IL-8, VEGF, and/or matrix metalloproteinase. Additional characteristics include, but are not limited to, at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more (or any range derivable therein) of the following: increase in expression or activity of cell cycle inhibitory proteins of p16, p38, p21, or p53; increase in disruption to downstream cell signaling cascades; persistent or increased DNA damage response (DDR); increased reactive oxygen species (ROS); appearance of heterochromatin condensation and rearrangement; altered expression of one or more Senescence-Associated Secretory Phenotype (SASP) cytokine; low energy metabolism; change in morphology (larger, flatter, highly granular); growth arrest in G0/G1; overexpression of a number of genes including, but not limited to, SM22, MMP1, and/or IFN-γ; deletion of mitochondrial DNA; telomere shortening; increase in lysosomal β-Gal activity; and, nuclear accumulation of G-Action and depolymerization of F-actin.

Pharmaceutical composition may comprise cells evaluated and/or determined to be senescent. In some cases, the senescent cells are determined to be senescent based on characteristics described herein.

Methods involving antigen presenting cells may further include obtaining the antigen presenting cells or precursors thereof from the patient. In some cases, the antigen presenting cells are dendritic cells, macrophages, or activated epithelial cells. In some cases, methods may involve differentiating precursors of antigen presenting cells into antigen presenting cells. The exposed antigen presenting cells may be administered in 1, 2, 3, 4, 5, 6 or more batches or doses. As discussed above, the patient may receive immunotherapy in conjunction with a composition that includes antigen presenting cells that have been exposed to induced senescent cells produced from cancer cells. In some cases, the immunotherapy is administered at the same time as the antigen presenting cells. In other cases, the immunotherapy is administered before the antigen presenting cells, while in others, immunotherapy is administered after the exposed antigen presenting cells.

Pharmaceutical compositions may have an additional immunotherapeutic agent. In some cases, the additional immunotherapeutic agent is an isolated tumor antigen. In further embodiments, the additional immunotherapeutic agent is an isolated antibody. In some embodiments, a composition comprises about, at least about, or at most about 10², 10³, 10⁴, 10⁵, 10⁶, 10⁷ 10⁸, 10⁹, 10¹⁰, 10¹¹, or 10¹² (or any range derivable therein) antigen presenting cells in methods and compositions.

In some cases there are pharmaceutical compositions made by a process comprising a) exposing cancer cells removed from a cancer patient to an effective amount of radiation and/or at least one senescence inducing agent to induce senescence; b) purifying or enriching for induced senescent cells; and c preparing a pharmaceutical composition of induced senescent cells.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Inhibition of poly(ADP-ribose) polymerase (PARP) combined with ionizing radiation (IR) delays tumor growth via inducing accelerated senescence of the tumor cells. (a) 5×10⁵ B16SIY murine melanoma tumor cells (B16) derived from C57BL/6 mice were inoculated subcutaneously, and after twenty-one days, the established tumors were treated with the PARP inhibitor veliparib (ABT-888, Abbott) twice daily starting 1 day before, and then daily after irradiation with IR at a dose of 6 Gray (Gy) or 12 Gy. Veliparib+IR treated tumors showed significant growth delay when compared to those treated with 6 Gy or 12 Gy IR alone, p=0.033, p=0.004. n=5-25/group. (b) Tumors treated as above were collected at 7 days following IR, either fixed/embedded for H/E staining (upper four images) or snap frozen for senescence-associated betagalactosidase (SA-β-Gal) staining (lower four images). Scale bars, 50 μm. (c) B16 cells were treated with veliparib+12 Gy in vitro and incubated 7 days, and then subjected to sorting via flow cytometry, based on separating populations with distinct forward scatter (size, FSC) and side scatter (granularity, SSC). When mice were injected with large (high FSC, high SSC) senescent cells in comparison to the small (low FSC, low SSC) non-senescent, proliferative cells, the large senescent cells (SC) failed to form tumors, while small non-senescent cells (NC) formed tumors readily. Coinjection of increasing proportions of senescent cells increasingly inhibited the growth of untreated cells. n=5-10/group.

FIG. 2. PARP inhibition modifies immuno-regulatory cytokine components in irradiated B16 tumor cells. (a) Correlation of expression of interferons, chemokines and other immune cell to cell signaling genes with senescent cell cycle arrest associated genes in tumor samples collected from experimental mice analyzed by RT-PCR and normalized with GAPDH. (b) Immunohistochemistry showing IFNβ, CXCL9, CXCL10 and CCL2 staining in large senescent tumor cells present in tumors treated with veliparib+IR. Data are representative of 5 experiments. Scale bars, 50 μm.

FIG. 3. CD8⁺ cells inhibit the growth of bystander non-senescent cells. (a) CD8⁺ cells contribute to irradiation effect and tumor growth delay following veliparib+IR. Mice bearing established tumors were treated with veliparib and 12 Gy and with reagents to deplete CD4⁺ T cell, CD8⁺ T cell, NK or macrophage cells. Depletion of CD8⁺ T cells abrogated the tumor growth delay following veliparib+12 Gy, p=0.003. Depletion of NK cells partially reduced the anti-tumor effect of veliparib+12 Gy, p=0.009. n=5-15/group. (b) CD8⁺ cells contribute to IR effect and tumor growth delay post veliparib+IR treatments. n=6-15/group. (c) CD8⁺ T cells maintain the tumor remission following veliparib+IR treatment, as illustrated by the decreased SA-βGal staining and increasing cellularity in CD8⁺ T cell depleted tumors.

FIG. 4. Senescent B16 tumor cells enhanced murine bone marrow-derived dendritic cell precursor (BMDC) proliferation, maturation and function to stimulate Th1 response. (a) Coculture with veliparib+IR induced senescent B16 tumor cells promoted BMDC proliferation and maturation, demonstrated by the increased expression of MHC-II and CD86 on CD11c⁺ cells. More larger cells were expanded from smaller immature bone marrow cells which gave rise to CD11c⁺ DC. Data are representative of 4 experiments. (b) BMDC cultured with veliparib+IR induced senescent cells stimulated CD8⁺ cell proliferation as detected by CFSE dilution assay and increased IFNγ production. Data are representative of 3 experiments. (c) Veliparib+IR induced senescent B16 cell elicited an antigen specific antitumor response in draining lymph node (DLN) cells as analyzed by ELISA of IFNγ production after exposure to melanoma antigen gp100. Results are means of duplicate culture with DLN cells collected from 3 individual mice.

FIG. 5. PARP inhibition enhanced vaccine potency of irradiated tumor cells. (a) Vaccine effect of B16 cells treated with 6 or 12 Gy alone, veliparib alone or veliparib+6 or 12 Gy compared. Treated B16 cells were injected subcutaneously on the right leg of syngeneic C57BL/6 mice and 7 days later untreated B16 tumor cells were injected in the left leg and tumor formation was followed. Like untreated B16 tumor cells, B16 cells treated with veliparib alone displayed no vaccine effect. While injection of B16 cells treated with 6 or 12 Gy blocked tumor formation in a majority of mice, the veliparib+IR treated B16 cells displayed the strongest vaccine effect. (b) When cells treated with veliparib+IR were subjected to sorting via flow cytometry, based on populations with distinct forward scatter (size, FSC) and side scatter (granularity, SSC), the vaccine effect was specific to the large (high FSC, high SSC) senescent cells and absent from the small (low FSC, low SSC) proliferative cells. (c) Veliparib+IR induced senescent p1048 murine pancreatic tumor cell elicited a more robust vaccine effect compared to p1048 tumor cells IR alone or untreated. (d) Veliparib+IR treated non-senescent TUBO murine mammary tumor cells failed to prevent tumor formation after injection of untreated TUBO cells.

FIG. 6. Senescent tumor cells delay the outgrowth of transplanted tumors and potentiate the effects of irradiation, by delaying tumor relapse after IR. (a) 5×10⁵ B16 tumor cells were inoculated subcutaneously on the right leg of syngeneic C57BL/6 mice. After 7 days, the emerging tumors were treated with injection of sorted large senescent tumor cells on the left leg. Significant growth delay was observed when compared to control (p=0.038). Some tumors were treated with 20 Gy, the addition of senescent tumor cells in a remote site delayed tumor growth following IR (p=0.003, n=5/group). (b) The size of tumors surgically removed from different treatment groups can be visualized. (c) FACS analysis of tumor infiltrating CD8⁺ cells reveals increased proportion of IFNγ positive cells when tumors were treated with senescent cell vaccine or IR, and a compound effect when treated with senescent cell vaccine and then IR.

FIG. 7. Identification of human cells induced to perform accelerated senescence via detection of senescence associated beta-galactosidase (SA-βGal) by DDAO-G red fluorescent substrate. (a) Flow cytometry of viable cells comparing SA-βGal (B-Gal) vs. side scatter (SSC-A), with senescent gate shown (1.6%). (b) Untreated cells; senescent gated cells (grey) overlaid with total cell population (black) showing forward scatter (size, FSC) vs. side scatter (granularity, SSC) distribution. (c) Viable veliparib+IR treated cells; B-Gal vs. SSC, with senescent gate shown (20%). (d) Veliparib+IR cells; senescent gated cells (grey) overlaid with total cell population (black), FSC vs. SSC distribution. Within the region shown by the black rectangle, 41%/of cells are B-Gal^{high} and 59% are B-Gal- or B-Gal^{low}.

FIG. 8. Glucose limitation affects IR-induced foci (IRIF) persistence and senescence in MCF7 cells expressing a GFP fusion to the 53BP1 IRIF binding domain as a reporter (MCF7^{Tet-On} GFP-IBD). Using GFP fluorescence to detect IRIF, cells displayed IRIF at 3 hours after 6 Gy irradiation that resolved more rapidly by 24 hours in cells growing in high glucose (4.5 g/l) media than in low glucose (1 g/l) media. Glucose limitation significantly increased IRIF persistence at 24 hours, based on measuring number of IRIF per cell. Mean IRIF per cell±SEM at 24 h were 8±0.3 for high glucose media and 17±0.9 for low glucose media, P value<0.0001. As shown in left-most images, irradiated cells growing in low glucose media develop senescent morphology and increased SA-βGal activity.

FIG. 9. Glycolysis inhibitors overcame the intrinsic radioresistance and induced IRIF persistence in radiation resistant PANC02 mouse pancreatic and U87 human glioma cell lines. PANC02^{Tet-On} GFP-IBD and U87^{Tet-On} GFP-IBD cells expressing the GFP-53BP1 IRIF reporter show pan-nuclear fluorescence before IR treatment and resolve most of the IRIF at 24 h after 6 Gy irradiation. Treating the cells with small molecule glycolysis inhibitors targeting glucose transport (Glut1i), hexokinase (HXi), pyruvate kinase (PKi), and lactate dehydrogenase (LDHi) markedly increased IRIF persistence at 24 hours in both IR resistant cell lines.

FIG. 10. Glycolysis inhibitor 2-deoxy-D-glucose (2DG) combined with irradiation increases cancer cell senescence in vivo in IR-resistant tumor xenografts. In tumors exposed to irradiation alone we did not observe any SA-βGal positive cells. Irradiation combined with glycolysis inhibitor 2DG induced numerous cells that stained positive for SA-βGal, even more then irradiation combined with PARP inhibitor veliparib (positive control). The strongest induction of SA-βGal was observed in irradiated tumors treated with 2DG and veliparib. These data indicate that glycolysis inhibitors may cooperate with PARP inhibitors to promote accelerated senescence in IR-resistant tumors.

FIG. 11. (a) TUBO murine mammary tumor cells propagated in 1 g/l glucose cell culture media and treated with veliparib+IR prevented tumor growth in mice. (b) TUBO cells growing in 1 g/l glucose media showed enhanced SA-βGal staining when treated with veliparib+IR over cells grown at 4.5 g/l glucose.

FIG. 12. Glucose restriction induced an altered senescence associated secretory phenotype pattern (SASP) and cell surface antigen expression in senescent TUBO cells induced in low (1 g/l) glucose media. (a) TUBO cells cultured in low or high glucose media were treated with veliparib+6 Gy or 6 Gy alone. At day 7 tumor cells were analyzed for senescent marker p21 and cytokine/chemokine expression by qRT-PCR. Relative gene expression was compared. (b) Kinetics of gene expression of TUBO cells treated with veliparib+6 Gy which were cultured in low or high glucose media.

FIG. 13. Irradiated senescent TUBO cell vaccine synergized with synthetic adjuvant CpG and IR to prevent tumor growth post IR in syngeneic Balb/c and autochthonous tumor-forming, tolerized Balb-NeuT mice. (a) TUBO cells cultured in low or high glucose media were treated with veliparib+6 Gy or 6 Gy alone and inoculated subcutaneously on the leg. Cells from draining lymph nodes (DNLs) were isolated and cultured with HER2 peptide or TUBO lysate for 5 days. Culture supernatants were collected and IFNγ secretion was tested using ELISA. (b) TUBO tumors were established in syngeneic mice on the right leg. Senescent TUBO cells were obtained by treatment cells with veliparib+6 Gy in low glucose media. At day 21 and 28 after tumor cell inoculations, 5×10⁵ senescent cells were inoculated in the left leg as vaccine. At day 28, tumors on the right leg also received 15 Gy IR. Tumors were measured and calculated as tumor volume (n=5). Arrows indicated times when vaccine cells and/or IR were given.

FIG. 14. Irradiated senescent TUBO cell vaccine prevents tumor growth in Balb/NeuT mice. (a) Vaccination of young Balb-NeuT mice with senescent TUBO cells propagated in low glucose media and treated with veliparib+IR in mice reduced the number of tumors developed. Combination with CpG further enhanced the vaccine effect in this model. Combination of vaccine cells+CpG with local IR enhanced the tumor growth delay. Ratios of CD8⁺ cytotoxic T cells to CD4⁺CD25⁺FoxP3⁺ regulatory T cells or CD11b⁺Gr1⁺ myeloid derived suppressor cells in CD45⁺ tumor infiltrating lymphocytes were shown. Values shown are sums of individually analyzed mice.

FIG. 15. Enhanced ionizing radiation induced foci (IRIF) formation as detected by immunofluorescence detection of phosphorylated H2AX (γH2AX) and of localization of 53BP1 protein and detection of accelerated senescence by senescence associated beta-galactosidase (SA-βGal) assay in B16SIY murine melanoma cells treated by veliparib and/or 6 Gy ionizing radiation.

FIG. 16. Flow cytometry based sorting of large senescent cells versus small non-senescent cells. B16 cells were treated with veliparib+6 Gy in vitro for 5 days and then subjected to sorting via flow cytometry, based on separating populations with distinct forward scatter (size, FSC) and side scatter (granularity, SSC). Sorted cell were reanalyzed by flow cytometry for their purity.

FIG. 17. Veliparib modifies the SASP in irradiated B16 tumor cells. (a) Kinetics of expression of cell to cell immune signaling mediators IFNβ. CCL5, and CXCL 11 correlated with induction of p21 as an indication of senescence development in B16 tumor cells treated with veliparib+IR. (b), (c) Induced expression of IFNβ and chemokine genes in B16 tumor cells induced by veliparib+IR treatment in vitro. (d) Veliparib accelerated cellular senescence in irradiated p1048 cells visualized by SA-βGal staining. (e) Higher IFNβ and chemokine gene expression in p1048 cells at 7 days after treatment with veliparib+IR.

FIG. 18. Flow cytometry analysis of tumor infiltrating lymphocytes (TILs) from B16 tumors treated with veliparib with or without irradiation. Greater numbers of IFNγ expressing CD8⁺ and NK cells were detected in veliparib+12 Gy treated tumors, suggesting an anti-tumor immune response.

FIG. 19. (a) Veliparib+IR treated senescent B16 tumor cell vaccines provide protection against tumor formation after challenge by injection of untreated B16 tumor cells, compared to vaccines prepared from B16 cells that were treated with either veliparib alone, IR alone or untreated. 5 days following vaccination, mice were injected with B16 tumor cells on the left leg. The percentage of tumor-free mice was followed. (b) Freeze thawed tumor cells have also been used in vaccine trials. To investigate the effect of freeze-thawing, untreated B16 cells, B16 cells treated only with IR and cells treated with veliparib+IR as for (a) were transferred between room temperature and liquid nitrogen for 5 cycles and then injected into the right leg. After 7 days, the mice were challenged with untreated B16 cells. Multiple cycles of freeze-thaw treatment markedly decreased the vaccine effect of both the IR and veliparib+IR treated cells.

FIG. 20. Drugs targeting chromatin modification and DNA repair enhanced radiation induced persistence of GFP-53BP1 foci as a reporter of IRIF in MCF7^{Tet-on} GFP-IBD human breast cancer cell line. (a) PARP inhibitor (PARPi) veliparib, histone deacetylase inhibitor (HDACi) SAHA (vorinostat, suberoylanilide hydroxamic acid), and histone acetyl transferase (Tip60) inhibitor (HATi) anacardic acid enhance radiation induced persistence of GFP-53BP1 foci MCF7 cells. (b) Compared to veliparib or radiation alone, veliparib+6 Gy promotes persistence of GFP-53BP1 foci, induces accelerated senescence and causes growth suppression in MCF7. (c) Veliparib enhances radiation induced senescence in different human cancer cell lines, including breast, prostate, melanoma and head and neck squamous cell cancer cell lines.

FIG. 21. Combining chemotherapy agents with veliparib induced accelerated senescence. (a) Cisplatin induced persistence of GFP-53BP1 foci in MCF7^{Tet-on} GFP-IBD cell line, resulting in accelerated senescence and growth suppression. Veliparib enhances this effect. (b) Fluorouracil (5-FU) enhances IRIF persistence and accelerates senescence in MCF7 cell line.

FIG. 22. Glucose metabolism inhibitors induced senescence in irradiated tumor cells. (a) 2-deoxyglucose induced persistence of GFP-53BP1 foci following senescence in irradiated MCF7^{Tet-on} GFP-IBD cells. (b) Glycolysis inhibitors including Glut1 inhibitor (Glut1i) phloretin (Phlo), hexokinase inhibitor (HXKi), pyryuvate kinase inhibitor (PKi) oxaloacetate, lactate dehydrogenase inhibitor (LDHi) oxamate and TCA cycle inhibitor (TCAi) dichloroacetic acid (DCA) all induced persistence of GFP-53BP1 foci following irradiation and promoted accelerated senescence in MCF7 cells. (c) Adenosine Monophosphate-Activated Protein Kinase (AMPK) activators metformin and compound C induced persistence of GFP-53BP1 foci after irradiation and promoted accelerated senescence in MCF7 cells.

FIG. 23. Senescence in hormone dependent tumors. (a) Tamoxifen induced persistence of GFP-53BP1 foci after irradiation and promoted accelerated senescence in MCF7 cell line. (b) Veliparib overcomes the activity of estrogen by promoting persistence of GFP-53BP 1 foci and inducing accelerated senescence in irradiated MCF7 cells.

FIG. 24. Immunostimolatory effect of senescent TRAMP-C2 cells obtained with combined IR(6Gy)+25 μM veliparib assessed as increased population of Cd11c positive cells—characteristics of differentiated DC.

### DESCRIPTION OF ILLUSTRATIVE EMBODIMENTS

### I. Methods and Compositions Involving Induced Senescent Cells

As detailed in this application, it was discovered that cancer cells treated er vivo to induce accelerated senescence have an anti-tumor vaccine effect and produce a robust adaptive immune anti-tumor response that prevents new tumor growth and potentiates radiation to reduce or eliminate established tumors.

In one embodiment, a cancer cell is obtained from a subject. Optionally, cells obtained from the subject can be expanded to increase their number, by methods known to one of skill in the art. The obtained cells are then treated with a senescence inducing agent, radiation, or a combination thereof to induce senescence in at least some of the cells. After such treatment, in some embodiments, senescent cells are sorted or purified. The treated cells are then reintroduced into the subject. Optionally, radiation therapy, either traditional or stereotactic body radiation therapy (SBRT), is then used at sites of remaining cancer in the subject.

In some embodiments, SBRT is used in combination with the methods and compositions described herein. SBRT delivers highly focused, high-dose radiation treatments in few fractions. SBRT aims to achieve the optimal therapeutic ratio by increasing the dose delivered to the tumor while minimizing normal tissue toxicity by reducing the volume of such tissue irradiated with this high dose. Reconstruction of the tumor volume using high quality images enables 3-D analyses and precise treatment planning. SBRT radiation fields are only slightly larger than the gross tumor volume and steep dose gradients tightly conform to the tumor. Consequently, higher doses of radiation can be delivered to the tumor in a single treatment, and fewer fractions are required to achieve a biologically effective dose. SBRT typically utilizes ablative ranges of radiation doses (≧10Gy/fraction) with a biologically effective dose of ≧45-100Gy. Fowler et al. have compared the theoretical relative biological effectiveness of conventional fractionated dose regimes and SBRT regimes (Fowler 2005). An SBRT schedule in the range of 45-69 Gy in 3-5 fractions was expected to have at least twice the relative biological effectiveness in non-small cell lung cancer as a conventional fractionated schedule of 60-70 Gy in 30-35 fractions.

As described in the examples, B16SIY (B16) murine melanoma in syngeneic mice was irradiated and treated with the poly(ADP-ribose) polymerase inhibitor (PARPi) veliparib to inhibit DNA repair, promote accelerated senescence and modulate inflammatory signaling. Senescent cells induced by radiation and veliparib express immunostimulatory cytokines, which in turn activate CTLs to drive an effective anti-tumor response.

Surprisingly, it was discovered that injecting senescent B16 cells as a therapeutic vaccine into tumor-bearing mice induced an anti-tumor CTL response and potentiated radiation to eliminate tumors in these mice. It is envisioned that this type of radiation-inducible immunotherapy may enhance radiotherapy responses to prevent local recurrence and metastasis in humans.

As demonstrated in the Examples, veliparib combined with IR achieves radiosensitization in a B16 melanoma model through the induction of senescence characterized by a modified, immunostimulatory senescence-associated secretory phenotype (SASP). Inoculation of mice with senescent B16 tumor cells prevented growth of new tumors after injection of untreated B16 cells at distant sites and dramatically sensitized established B16 tumors to radiation.

It was further discovered that treatment of the P1048 murine pancreatic adenocarcinoma with veliparib and radiation resulted in accelerated senescence, and that these cells served as an effective vaccine against subsequent challenge with untreated P0148 cells.

It is envisioned that any cancer cell in which accelerated senescence can be induced by treatment with a PARP inhibitor and/or radiation is suitable for use in the methods and compositions discussed herein. Cancer broadly refers to cellular-proliferation and/or cellular growth disease states. Cancer may also refer to a recurring cancer, a cancer metastasis, any pre-cancerous cell or cell in a pre-cancerous state, a neoplasm, any therapy resistant cancer or any cancer previously treated by chemotherapy, radiotherapy, surgery or gene therapy. The cancer may be breast, prostate, ovarian, brain, melanoma, colorectal, liver, lymphoma, lung, oral, throat, head, neck, nasal or paranasal, spleen, lymph node, small intestine, large intestine, blood cells, esophageal, stomach, pancreatic, endometrial, testicular, prostate, ovarian, skin, esophageal, bone marrow, heart, blood, cervical, bladder, kidney, urethral, thyroid, glioma, and/or gastrointestinal cancers. Cancer also includes but is not limited to: sarcoma, myxoma, rhabdomyoma, fibroma, lipoma and teratoma, bronchogenic carcinoma, alveolar (bronchiolar) carcinoma, bronchial adenoma, tumors of the parotid, chondromatous hamartoma, mesothelioma, squamous cell carcinoma, leiomyosarcoma, carcinoma of the stomach, pancreatic ductal adenocarcinoma, insulinoma, glucagonoma, gastrinoma, pancreatic carcinoid tumors, vipoma, cancers of the small bowel cancers of the large bowel, colorectal adenocarcinoma, kidney adenocarcinoma, renal cell carcinoma, Wilm's tumor, nephroblastoma, bladder and urethra carcinomas, prostate adenocarcinoma and sarcoma, testicular cancers, hepatoma, hepatocellular carcinoma, cholangiocarcinoma, hepatoblastoma, angiosareoma, hepatoceltular adenoma, hemangioma, osteosarcoma, fibrosarcoma, malignant fibrous histiocytoma, chondrosarcoma, malignant lymphoma (reticulum sarcoma), multiple myeloma, Ewing's sarcoma, malignant giant cell tumor chordoma, osteochrondroma (osteocartilaginous exostoses), benign chondroma, chondroblastoma, chondromyxofibroma, osteoid osteoma and giant cell tumors; granuloma, xanthoma, osteitis defornians, meningioma, meningiosarcoma, gliomatosis, astrocytoma, medutloblastoma, glioma, ependymoma, germinoma; pinealoma; glioblastoma, multiformae, oligodendroglioma, schwannoma, retinoblastoma, congenital tumors, neurofibroma, endometrial carcinoma, cervical carcinoma, pre-tumor cervical dysplasia, ovarian carcinoma; serous cystadenocarcinoma, mucinous cystadenocarcinoma, unclassified carcinoma; granulosa-theca cell tumors, Sertoli Leydig cell tumors, dysgerminoma, malignant teratoma, vulvar cancer, vaginal cancer, fallopian tube carcinoma, chronic and acute myeloid leukemia, acute lymphoblastic leukemia, chronic lymphocytic leukemia, myeloproliferative diseases, multiple myeloma, myelodysplastic syndrome, Hodgkin's disease, non-Hodgkin's lymphoma, malignant lymphoma, endothelioma, malignant melanoma, basal cell carcinoma, squamous cell carcinoma, Kaposi's sarcoma, moles dysplastic nevi, lipoma, angioma, dermatofibroma, keloids, psoriasis, germ cell tumors, myelodysplastic and myeloproliferative disorders and neuroblastoma. In other embodiments, the methods and compositions described herein may be used to treat benign tumors, keloid, neoplasia, dysplasia, metaplasia, hyperplasia, preneoplastic cells, transformed cells, precancerous cells, carcinoma in situ, cervical intraepithelial neoplasia (CIN), ductal carcinoma in situ (DCIS) and related conditions. For example, the methods may be used to treat any of the cancers discussed herein, including pre-cancers, as well as other cancers not discussed herein.

Furthermore, it is envisioned that immunologically regulated cancers, such as head and neck cancer, renal cell carcinoma, and melanomas are highly susceptible to senescence induction by treatment with a senescence inducing agent and/or radiation. Head and neck cancer includes a group of biologically similar cancers that originate in the upper respiratory and digestive tracts. Squamous cell carcinomas of the head and neck (SCCHN) originate from the mucosal epithelium and represent approximately 90% of all head and neck cancers. Head and neck cancers are frequently aggressive and often spread to the lymph nodes. These cancers are commonly treated with surgery and potentially combined with chemotherapy and radiation. Renal cell carcinoma (RCC) originates in the lining of the proximal convoluted tubules of kidneys and is the most common type of kidney cancer in adults, representing approximately 80% of all cases. RCC is the most lethal of all genitourinary cancers and is commonly treated with surgery. It is currently resistant to radiation and chemotherapy, while sometimes responsive to immunotherapy. Melanoma is another example of immunoresponsive cancer and it consists of a malignancy of melanin pigment producing melanocytes predominantly found in skin. While melanomas are not the most common type of skin cancer, they cause approximately 75% of all deaths related to skin cancer. The treatment consists of surgery combined with chemotherapy, immunotherapy, and radiation. Whether cells are capable of undergoing accelerated senescence may be assessed using any suitable method, including those described herein. For example, cells may be assessed for accelerated senescence by observing whether the cells exhibit the characteristic morphology, SA-βGal expression, or increased expression of cytokines characteristic of senescence.

In addition to veliparib, it is envisioned that other agents capable of inducing senescence in cancer cells may be used in the methods provided herein. Such senescence inducing agents include, without limitation, tumor-suppressor inducers, such as esophageal cancer-related gene 4 (Ecrg4) inducers, p16 (CDKN2A) inducers, p53 (p53) inducers, Rb (Rb) inducers; mitosis inhibitors, such as discodermalide, taxol, vincristine, and Aurora A kinase inhibitors; nucleic acid damage inducing and interfering agents, such as alkylating agents and antimetabolites (purine and pyrimidine analogues, antifolates); antitumor antibiotics; topoisomerase inhibitors; hormone and growth factor inhibitors (e.g., Tamoxifen) and PARP inhibitors (Xue 2007, Rakhra 2010) Examples of suitable PARP inhibitors include, but are not limited to, BSI-201, olaparib, iniparib, AGO14699, MK4827, KU-0059436, CEP9722, LT-673, and 3-aminobenzamide. Suitably, the PARP inhibitor has a Ki of 1 μM or less with respect to PARP-1 or an IC₅₀ of 100 μM or less. In some embodiments, the PARP inhibitor has a Ki or IC₅₀ in the nanomolar range.

It is envisioned that inhibitors of histone acetyltransferases (HATs), histone deacetylase (HDACs), DNA methyltransferases (DNMTs) and demethylases, poly(ADP-ribose) polymerase (PARP), histone ubiquitylase and deubiquitination enzymes, histone chaperones, histone exchange complexes and chromatin remodelers may be suitable senescence inducing agents. Suitable HDAC inhibitors, include, for example, butyrate, valproic acid, trichostatin A (TSA), and suberoylanilide hydroxamic acid (SAHA). Suitable DNMT inhibitors include, for example, azacytidine, decitabine, disulfiram, and zebularine.

Other potential senescence inducing agents include inhibitors of the NAD+ salvage pathway, including nicotinamide (NAM) and inhibitors of nicotinamide phosphoribosyltransferase (NAMPT), such as [N-[4-(1-benzoyl-4-piperidinyl)butyl]-3-(3-pyridinyl)-2E-propenamide (FK866) and (E)-1-[6-(4-chlorophenoxy)hexyl]-2-cyano-3-(pyridin-4-yl)guanidine (CHS 828).

It is further envisioned that low glucose cell growth conditions (glucose limitation) and compounds targeting glycolytic metabolism of tumors may also be suitable senescence inducing agents, including: glucose transporter inhibitors (e.g., 2-deoxyglucose, phloretin, silybin/silibinin, Glut1 inhibitors, etc.); hexokinase 2 inhibitors (e.g., 2-deoxyglucose, lonidamine, bromopyruvic acid, etc.); phosphofructokinase 2 inhibitors; phosphofructo-2-kinase/fructose-2,6-bisphosphatase 3 inhibitors (3PO 3-(3-pyridinyl)-1-(4-pyridinyl)-2-propen-1-one, etc.); pyruvate kinase (PK) inhibitors (e.g., oxaloacetate, etc.) and pyruvate kinase M2 inhibitors (e.g., TLN-232/CAP-232 (peptidic inhibitor), Shikonin and alkannin, etc.); lactate dehydrogenase (LDH) inhibitors (e.g. oxamate, etc.) and LDH5 lactate dehydrogenase 5 inhibitor (Gossypol/AT-101 (Malarial LDH inhibitor), FX11, etc.); and carbonic anhydrase-9 inhibitors (Indisulam, Girentuximab, etc.); activators of oxidative phosphorylation and pyruvate dehydrogenase (PDH) complex activators (e.g., pyruvate dehydrogenase kinase inhibitors (dichloroacetate (DCA)), etc.); methylpyruvate; membrane-bound V-ATPase inhibitors (e.g., esomeprazole, etc.); monocarboxylate transporter 1 inhibitors (e.g., AZD3965, etc.); Adenosine Monophosphate-Activated Protein Kinase activators (AICAR (5-aminoimidazole-4-carboxamide 1-D-ribonucleoside), Metformin, phenformin, A769662, thia-zolidinediones (TZDs), RSVA314, RSVA405, etc.); and hypoxia-inducible factor-1 inhibitors (e.g., BAY87-2243, EZN-2968 (Antisense oligonucleotide), Compound C, etc.). Additional senescence inducing agents may include glutamine combining with glucose limitation and compounds affecting glutamine metabolism and hexosamine biosynthesis, including: dimethyl 2-oxoglutarate (membrane-permeant alpha-ketoglutarate analog); glutamine:fructose amidotransferase (GFAT) inhibitors (e.g., DON (6-diazo-5-oxo-L-norleucine); uridine diphospho-N-acetylglucosamine:polypeptide beta-N-acetylglucosaminyltransferase (OGT) inhibitors (e.g., alloxan, azaserine, etc.); inhibitors of N-acetyl-glucosamine; and inhibitors of glutamate dehydrogenase (GDH) activity (epigallocathenin gallate (EGCG)). Other suitable senescence inducing agents may include small molecule inhibitors of a SCF-type ligase or its components (e.g., Bortezomib (also known as Velcade or PS-341) the class of general proteasome inhibitor; MLN4924, a small molecule inhibitor of NEDD8-activating enzyme.

Activators of WT p53 or reactivators or inhibitors of mutant p53 may also be used as senescence inducing agents. Examples of WT p53 activators include Nutlin-3, RITA, MI-219, BDA, HL198C, Tenovin-1, JJ78:12. Mutant p53 reactivators include CP31398, PRIMA-1, MIRA-1, Ellipiticine, p53R3, WR1065. Mutant p53 inhibitors (e.g., RETRA) may be used.

In the examples below, treatment of cells with 10 μM veliparib and 6 or 12 Gy radiation or radiation alone was found to induce accelerated senescence. It is envisioned that any dosage of IR capable of inducing accelerated senescence alone or in combination with any suitable concentration of a senescence inducing agent may be used in the methods of the invention provided that the IR or combination of IR and senescence inducing agent is capable of inducing senescence. The dosage of IR and concentration of PARP inhibitor may depend on the cell type and/or on the type of PARP inhibitor. It is expected that IR dosages of at least 2 Gy will be effective to induce senescence. Suitably, the IR dose is at least 6 Gy. In some embodiments the radiation dose used to induce senescence, either alone in or in combination with a senescence inducing agent or compound may have a lower limit of 0.5, 1, 1.5, 2, 2.5, 3, 3.5, 4, 4.5, 5, 5.5, 6, 6.5, 7, 7.5, 8, 8.5, 9, 9.5, or 10 Gy and an upper limit of 10.5, 11, 11.5, 12, 12.5, 13, 13.5, 14, 14.5, 15, 15.5, 16, 16.5, 17, 17.5, 18, 18.5, 19, 19.5 or 20 Gy. However, including an agent that induces senescence, such as a PARP inhibitor, may enhance efficacy, as was shown with the combination of IR and veliparib. Exposure to veliparib at a concentration of at least 100 nM in combination with IR would be expected to induce senescence. It is envisioned that any other treatment that can damage chromosomal DNA or disrupt chromatin integrity or induce other conditions known to those skilled in the art sufficient to promote cellular senescence including accelerated senescence, replicative senescence, stress-induced premature senescence (SIPS), therapy induced senescence (TIS), oncogene induced senescence (OIS) may be satisfactory. Further, it is envisioned that any treatment, such as infection with a virus, transfection with a gene, treatment with a protein, a peptide or a drug, that can alter the secreted proteins and cell surface proteins of cells that are rendered senescent, including danger signals, damage associated molecular pattern (DAMP), “eat-me” signals, “find-me” signals, senescence messaging secretome (SMS), senescence associated secretory phenotype (SASP), may also be used in the preparation of the senescent cells to enhance their vaccine properties.

As described in the Examples, injection of unsorted cancer cells treated with veliparib and IR afforded some protection against tumor regrowth, metastasis, and/or challenge with untreated cells. However, sorting the treated cells to obtain a fraction enriched for senescent cells produces a vaccine with enhanced efficacy and reduces the risk of introducing active cancer cells into the subject. Therefore, in one embodiment, methods may involve the further process of separating treated cells to increase the concentration senescent cells relative to non-senescent cells. In some embodiments the preparation is substantially free of non-senescent cells, i.e., non-senescent cells comprise less than 10%, 5%, 1%, or 0.1% of the total cell population. Suitably, the ratio of senescent to non-senescent cells is in the range of from about 2:1-10,000, and at least 2:1, 3:1, 4:1, 5:1, 6:1, 7:1, 8:1, 9:1, 10:1, 12:1, 15:1, 20:1, 50:1, 100:1, or 1000:1.

In certain aspects it is desirable to expand or maintain cells prior to senescence induction or subsequent to senescence induction. In some embodiments, cancer or tumor cells in which senescence will be induced may be expanded, cultured or maintained for some amount of time prior to induction of senescence. Cells in which senescence has been induced may be maintained for some amount of time before administration to a subject. Standard methods used in tissue culture generally are described in Animal Cell Culture (1987); Gene Transfer Vectors for Mammalian Cells (1987); and Current Protocols in Molecular Biology and Short Protocols in Molecular Biology (1987 & 1995) which are herein incorporated by reference.

It is envisioned that the cells may be sorted and analyzed by any suitable cell isolation and sorting technology, including, and not limited to, manual selection, size-based filtering, antibody-based sorting, magnet-based sorting, microfluidic sorting, micromechanical valve-based chip sorting, dielectrophoretic sorting, laser-capture microdissection, and fluorescence-based sorting.

The senescent sorted cells may be obtained by treating cells with a senescence inducing agent and/or radiation and sorting them according to size or granularity based upon forward and side scatter to isolate populations of large (enriched for senescent) or small (enriched for nonsenescent) cells. Sorted cells may be further analyzed by flow cytometry to demonstrate the enrichment, using fluorescently-labeled antibodies for tumor cell surface antigen markers, DNA damage response markers, danger signals, senescent cell surface antigen markers, cytokine receptors, and/or intracellular cytokines.

Senescent cells can be identified and sorted or purified based upon one of their salient features. It is contemplated that at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more of these characteristics may be evaluated in embodiments discussed herein in order to determine that a cancer cell has been induced into senescence. It is specifically contemplated that one or more of these characteristics may also be excluded as a way to evaluate senescence.

In one embodiment, senescent cells are sorted or purified based upon their increase in size. Senescent cells are enlarged relative to the size of non-senescent counterparts, sometimes enlarging more than twofold relative to the size of nonsenescent counterparts and exhibiting characteristic large, flattened cell shape. In another embodiment, senescent cells are sorted or purified based upon their expression of p16INK4a, which is not commonly expressed by quiescent or terminally differentiated cells. Alternatively, increased p21Cip1 expression may be used as a marker of senescence. In yet other embodiments, senescent cells are sorted or purified based upon their expression of β-galactosidase, or the increase in lysosomal mass. In additional embodiments, senescent cells are sorted or purified using fluorescent β-galactosidase substrate 9H-(1,3-Dichloro-9,9-Dimethylacridin-2-One-7-yl)B-D-Galactopyranoside (DDAO-Galactoside, fluorescent β-galactosidase substrate 5-Dodecanoylaminofluorescein Di-β-D-Galactopyranoside (C12FDG), and colorimetric β-galactosidase substrate 5-Bromo-4-Chloro-β-Indolyl β-D-Galactopyranoside (X-Gal). In a further embodiment, senescent cells are sorted or purified based upon nuclear loci of persistent DNA damage response also called ionizing radiation induced foci (IRIF).

Additional characteristics of senescence include, but are not limited to: an increase in expression or activity of cell cycle inhibitory proteins of p16, p38, p21, or p53 (Campisi 2012, which is hereby incorporated by reference); increase in disruption to downstream cell signaling cascades (Campisi 2012, which is hereby incorporated by reference); persistent or increased DNA damage response (DDR) (Campisi, 2012, which is hereby incorporated by reference); increased reactive oxygen species (ROS) (Campisi, 2012, which is hereby incorporated by reference); appearance of heterochromatin condensation and rearrangement (Campisi, 2012, which is hereby incorporated by reference); altered expression of one or more Senescence-Associated Secretory Phenotype (SASP) cytokine (Coppe, 2010, which is hereby incorporated by reference); low energy metabolism (Toussaint, 2000, which is hereby incorporated by reference); change in morphology (larger, flatter, highly granular) (Toussaint, 2000, which is hereby incorporated by reference); growth arrest in G0/G1 (Toussaint, 2000, which is hereby incorporated by reference); overexpression of a number of genes including, but not limited to, SM22, MMP1, and/or IFN-γ (Toussaint 2000, which is hereby incorporated by reference); deletion of mitochondrial DNA (Toussaint, 2000, which is hereby incorporated by reference); telomere shortening (Toussaint, 2000, which is hereby incorporated by reference); increase in lysosomal β-Gal activity (Lee, 2006, which is hereby incorporated by reference); and, nuclear accumulation of G-Action and depolymerization of F-actin (Kwak, 2004, which is hereby incorporated by reference).

In particular embodiments, an increase in expression and/or activity of 1, 2, 3, 4, 5, 6, 7, 8, 9, 10 or more (or any range derivable therein) of the following may be measured or evaluated or be the basis for determining senescence: IL-6, IL-7, IL-1a, IL-1b, IL-13, IL-15, IL-8, GRO-a,GRO-b, GRO-g, MCP-2, MCP-4, MIP-1a, MIP-3a, HCC-4, Eotaxin-3, GM-CSF, MIF, Amphiregulin, Epiregulin, Heregulin, EGF, bFGF, HGF, KGF (FGF7), VEGF, Angiogenin, SCF, SDF-1, PIGF, IGFBP-2, IGFBP-3, IGFBP-4, IGFBP-6, IGFBP-7, MMP-1, MMP-3, MMP-10, MMP-12, MMP-13, MMP-14, TIMP-2, PAI-1, PAI-2, tPA, uPA, Cathepsin B, ICAM-1, ICAM-3, OPG, sTNFRI, TRAIL-R3, Fas, sTNFRII, Fas, uPAR, SGP130, EGF-R, PGE2, Nitric oxide, or Fibronectin. In further embodiments, a decrease in expression and/or activity of TIMP-1 may be measured or evaluated or be the basis for determining senescence. In some embodiments, a change in expression and/or activity of 1, 2, or 3 of the following may be measured or evaluated or be the basis for determining senescence: Reactive oxygen species, Collagen, or Laminin. In some embodiments, one or more of the following are not used as a marker for senescence: TECK, ENA-78, I-309, I-TAC, Eotaxin, G-CSF, IFN-gamma, BLC, and/or NGF.

In other embodiments, senescent cells are sorted or purified based upon a SASP phenotype that can affect the behavior of neighboring cells. Many SASP factors are secreted by senescent cells, including amphiregulin and growth-related oncogene (GRO) α, interleukin 6 (IL-6) and IL-8, VEGF, and matrix metalloproteinases. Additionally, senescent cells may be sorted or purified by any combination of the foregoing, as well as by any method that would be known to one of ordinary skill in the art.

In the Examples, the senescent cells were administered by intramuscular injection. However, it is envisioned that the vaccine may be administered by any suitable mode, including, for example, any enteral or parenteral mode, such as intravenous, subcutaneous, intratumor, and intraocular injections, or inhalation.

A composition for administration may be formed by combining the treated cancer cells with any suitable pharmaceutical carrier. In certain aspects the senescent cells are not subjected to multiple cycles of freeze-thawing or other treatments such as detergents, heating, hypotonic solutions, or mechanical disruption that cause loss of cell integrity and metabolic activity.

The vaccine may be administered alone, or in combination with IR. The vaccine may be administered before, during, or after administration of IR. In the examples below, the vaccine was administered to mice at a dose of 5×10⁵ cells per animal. It is envisioned that dosages of at least about 10⁴ cells would be needed to treat human subjects. Suitably, at least about 10⁶ to 10⁹ cells would be used. In certain aspects 10³, 10⁴, 10⁵, 10⁶, 10⁷, 10⁸ or 10⁹ cells or any range within would be used.

As can be seen from the results reported herein, combining veliparib with ionizing radiation induces a robust anti-tumor effect in murine cancer models. This anti-tumor effect is mediated by the induction of accelerated senescence and modulation of the senescence-associated secretory phenotype (SASP) (Rodier 2009, Orjalo 2009) to activate an immune response characterized by CD8⁺ and NK cell-dependent tumor cytotoxicity. PARP inhibition by veliparib may have a direct role in both promoting senescence and altering the SASP following therapeutic radiation. Our findings indicate that inhibiting PARP and administering ionizing radiation promotes accelerated senescence and remodels the SASP, which induces an anti-tumor immune response.

Among the SASP components most affected by PARP inhibition, the immunostimulatory cytokine IFNβ was markedly induced by veliparib+IR as compared to IR alone. The significance of immune cell activation and trafficking associated with IFNβ signaling/production in the irradiated tumor microenvironment has been noted (Dunn 2006, Burnette 2011). For example, IFNβ induces expression of multiple cytokine/chemokines and reinforces tumor cell senescence (Novakova 2010). Taken together with prior work on IFNβ in radiation responses (Burnette 2011, Meng 2010) this establishes a link between senescence and increased IFNβ production, leading to enhanced priming and a more efficient host-cell IFNγ-mediated immune response.

A role for the innate immune system in eliminating senescent cells from tumors upon reexpression of p53 has been reported (Xue 2007). CD4+ T cells can mediate anti-tumor effects by inducing senescence in MYC-activated tumor cells (Rakhra 2010). Here, using murine tumors in syngeneic mice, we discovered a key role for CD8⁺ T cells in eliminating senescent tumor cells following irradiation and PARP inhibition. By inducing B16 tumor cell senescence and an altered SASP, veliparib+IR promoted dendritic cell proliferation maturation and function, which led to activation of tumor-specific IFNγ-expressing CD8+ T cells, each implicated as mediators of radiation response (Meng 2010, Lugade 2008, Lee 2009) and determinants of immunogenic tumor regression (Dunn 2006, Zhang 2008).

Importantly, we observed robust immune activation and resulting anti-tumor effects induced by senescent cells, whether are formed in situ by irradiation of tumors in the presence of veliparib, or induced by veliparib and irradiation in vitro and then injected into mice to prevent new tumors or to potentiate irradiation of established tumors. These findings may have direct relevance to treatment of human cancer. Of immediate significance, we propose that the success of ongoing clinical trials of the PARP inhibitors olaparib and veliparib (Penning 2010) in combination with chemotherapy or radiation may depend more on driving accumulation of senescent cells to activate host anti-tumor responses than their effects on DNA repair per se.

It is envisioned that human cancer patients may be inoculated with senescent cells to target anti-tumor immune response to the primary tumor and/or gross metastases. This treatment may optionally be used in conjunction with radiotherapy. It is reasonably expected that using this method, one may obtain improved local control and by activating anti-tumor CTLs, reduced likelihood of new metastases. Success of such a radiation-inducible senescence-mediated immunotherapy would lead a paradigm shift in the use of ionizing radiation in treatment of advanced cancer from local therapy for tumor control to a systemic modality directed at cures.

It is envisioned that, in one embodiment, the invention is directed to a therapeutic composition and method for stimulating an immune response based on adoptive transfer. In such an embodiment, immune cells are obtained from a subject and exposed to senescent cells to induce a response, and such exposed immune cells are administered to the subject to induce an immune response against the cell type used to create the senescent cell. Immune cells appropriate for such an embodiment include a subject's bone marrow derived effector and precursor cells, dendritic cells, and T cells, as well as other cells known in the art.

It is further envisioned that in a particular embodiment, the immune cells are antigen presenting cells, such as dendritic cells (DC), and such cells are stimulated by exposing the cells in vitro to senescent cells prepared from cancer cells obtained from a subject. The exposed antigen presenting cells are then administered to the subject. It is further envisioned that in some embodiments the immune cells used may be T cells, such as CD45RA⁺ CD62L⁺ naive (TN) cells, CD45RO⁺ CD62L⁺ central memory (TCM) cells, and CD62L⁻ effector memory (TEM) cells, macrophages, epithelial cells, other antigen presenting cells, or other cells known in the art. In some embodiments, the immune cells are derived from the circulating blood or derived from the lymph nodes or derived from the bone marrow of the subject. In some embodiments, the immune cells may be derived from other cells such as adult stem cells, inducible pluripotent stem cells or other cells that are derived from the subject. In some embodiments, the immune cells may be expanded after isolation from the subject, and exogenous growth factors may be added. Additionally, the immune cells may be further engineered to enhance their immune activation or effector function.

The cells from a patient's cancer may be obtained from a tissue, such as a primary tumor, a locally spread tumor or a metastasis by surgical excision, by open biopsy, by needle biopsy, or obtained from a fluid including blood, lymph, cerebrospinal fluid, ascites fluid, pleural effusion, pericardial effusion, or by other means known to those skilled in the art. These cells could then be propagated and expanded in vitro or treated to render them senescent immediately. In one embodiment, a few cancer cells or even a single cancer cell might be propagated and expanded in vitro using methods such as conditional reprogramming of epithelial cells using Rho kinase inhibitor and a feeder layer (Liu 2012) or via induced pluripotent cell technology. It is further envisioned that, in one embodiment, the senescent cells derive from a cell line derived from a cell or from cells from a patient's cancer, obtained as above, where the cells have been modified to permit their growth in culture. To allow their growth as a cell line, a cancer cell or cells obtained as above may be infected with a virus, transfected with genes, or treated with proteins, peptides or drugs, to render them capable of growth in vitro. It is further envisioned that cells obtained from the cancer, propagated from these cells, or a cell line derived from these cells might be stored by freezing or other means to provide a means to derive senescent cells at a future time, as might be required to treat recurrence. It is further envisioned that, in one embodiment, an immortalized cell line derived from the patient or from their cancer that can be continuously expanded in vitro would be used. It is further envisioned that, to facilitate repeated treatment, this immortalized cell line would be stored by freezing or other means for repeated use. It is further envisioned that, in one embodiment, an immortalized cell line that can be continuously expanded in vitro while maintaining the specific genotype and phenotype properties required for the universal immune response and ex vivo stimulation of immune cells isolated from any subject. The exposed immune cells are then administered to the subject to induce an immune response against the cancer cells in the subject.

Additionally, it is envisioned that, in certain embodiments, the compositions and methods discussed can be utilized in combination with an immunotherapy. In some embodiments, immunotherapies are antibodies targeting factors involved in regulation of immune cells, including: CD11b, CD25, CD152 (cytotoxic T-lymphocyte antigen-4; CTLA-4), CD137 (4-1BB), CD134 (OX-40), and CD274 (programmed death ligand-1; PD-L1), as well as other targets known in the art. For example, CD137 stimulation results in enhanced expansion, survival, and effector functions of newly primed CD8⁺ T-cells, acting, in part, directly on these cells. While both CD4⁺ and CD8⁺ T-cells have been shown to respond to CD137 stimulation, enhancement of T-cell function is greater in CD8⁺ cells. In some embodiments, immunotherapies are co-stimulators of immune cell function or other immunotherapeutic strategies, such as Treg depletion, or blockade of PD-1 or IDO. It is contemplated that the combination may be administered to the patient concurrently (at the same time) and in the same composition, concurrently but in separate compositions, or serially.

In certain embodiments, the compositions and methods of the present invention involve a therapeutic composition that may be used in combination with other therapeutic strategies to treat cancer, such as surgery or chemotherapy. These combinations would be provided in a combination effective to achieve the desired effect. This process may involve providing chemotherapy in the same composition, concurrently but in separate compositions, or serially, or performing surgery at the same or different time as providing the therapeutic composition discussed herein.

It will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

In certain embodiments, compositions are providing comprising induced senescent cells or antigen presenting cells together with one or more of the following: a pharmaceutically acceptable diluent; a carrier; a solubilizer; and emulsifier; a preservative; and/or an adjuvant. Such compositions may contain an effective amount of induced senescent cells in the preparation of a pharmaceutical composition or medicament. Such compositions may be used in the treatment of cancer, as discussed herein.

The induced senescent cells or antigen presenting cells may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The form depends upon the mode of administration and the type of cancer being targeted. The compositions may also include pharmaceutically acceptable vehicles, carriers or adjuvants, well known in the art. Types of adjuvants include Freund's (complete and incomplete), saponins (e.g., Qui1A, QS21), muramyl dipeptides and derivatives (MTP-PE), copolymers, ISCOMS, cytokines, and oligonucleotides

A “pharmaceutically acceptable” vehicle, carrier or adjuvant is a nontoxic agent that can be tolerated by a recipient patient. Representative non-limiting examples of such agents include human serum albumin, ion exchangers, alumina, lecithin, buffer substances such as phosphates, glycine, sorbic acid, potassium sorbate, and salts or electrolytes such as protamine sulfate. Suitable vehicles are, for example, water, saline, phosphate-buffered saline, dextrose, glycerol, ethanol, or the like, and combinations thereof. Other suitable agents are well-known to those in the art. See, for example, Remington's Pharmaceutical Sciences, Mack Publishing Company, Easton, Pa., 19th edition, 1995. Actual methods of preparing such compositions are also known, or will be apparent, to those skilled in the art. See, e.g., Remington's Pharmaceutical Sciences, 1995, supra.

Acceptable formulation components for pharmaceutical preparations are nontoxic to recipients at the dosages and concentrations employed. In addition to the antibodies and antigen-binding regions that are provided, the compositions may contain components for modifying, maintaining or preserving, for example, the pH, osmolarity, viscosity, clarity, color, isotonicity, odor, sterility, stability, rate of dissolution or release, adsorption or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 1995, supra, hereby incorporated by reference in its entirety for all purposes).

The primary vehicle or carrier in a pharmaceutical composition may be either aqueous or non-aqueous in nature, though specific embodiments concern aqueous formulations containing cells. Suitable vehicles or carriers for such compositions include water for injection, physiological saline solution or artificial cerebrospinal fluid, possibly supplemented with other materials common in compositions for parenteral administration. Neutral buffered saline or saline mixed with serum albumin are further exemplary vehicles. Compositions comprising induced senescent cells or antigen presenting cells may be prepared for storage by mixing the selected composition having the desired degree of purity with optional formulation agents in the form of a lyophilized cake or an aqueous solution.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions may comprise TRIS buffer of about pH 6.5-8.5, or acetate buffer of about pH 4.0-5.5, which may further include sorbitol or a suitable substitute therefor.

The pharmaceutical composition to be used for in vivo administration typically is sterile. The composition for parenteral administration may be in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions may be administered using conventional modes of delivery including, but not limited to, intravenous, intraperitoneal, oral, intralymphatic, subcutaneous administration, intraarterial, intramuscular, intrapleural, intrathecal, and by perfusion through a regional catheter. Local administration to a tumor or tumor bed in question, will also find use in embodiments discussed herein. Eye drops may be used for intraocular administration. When administering the compositions by injection, the administration may be by continuous infusion or by single or multiple boluses. Intravenous injection provides a useful mode of administration due to the thoroughness of the circulation in rapidly distributing antibodies. For parenteral administration, cells may be administered in a pyrogen-free, parenterally acceptable aqueous solution comprising the cells in a pharmaceutically acceptable vehicle. A particularly suitable vehicle for parenteral injection is sterile distilled water in which the cells are formulated as a sterile, isotonic solution, properly preserved.

Once the pharmaceutical composition has been formulated, it may be stored in sterile vials as a solution, suspension, gel, emulsion, solid, or as a dehydrated or lyophilized powder

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

There are also kits for producing multi-dose or single-dose administration units. For example, kits may each contain both a first container having a aqueous diluent, including for example single and multi-chambered pre-filled syringes (e.g., liquid syringes, lyosyringes or needle-free syringes).

For purposes of therapy, cells are administered to a patient in a therapeutically effective amount. A “therapeutically effective amount” is one that is physiologically significant. An agent is physiologically significant if its presence results in a detectable change in the physiology or disease or disorder state of a recipient. A “prophylactically effective amount” refers to an amount that is effective to prevent, hinder or retard the onset of a disease state or symptom.

Therapeutically effective doses will be easily determined by one of skill in the art and will depend on the severity and course of the disease, the patient's health and response to treatment, the patient's age, weight, height, sex, previous medical history and the judgment of the treating physician. Typically, it is desirable to provide the recipient with a dosage of cells which is in the range of from about 1 pg/kg to 10 mg/kg (amount of agent/body weight of patient), although a lower or higher dosage also may be administered as circumstances dictate.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or cells/ml or cells/ml/kg or cells/ml/hour or any range derivable therein. Milligrams and micrograms refer to the weight of cells. Kg refers to the patient's weight. MI refers to the volume of the composition containing the therapeutic agent. Minutes and hours in the context of a weight or volume refers to infusion rate.

In certain embodiments, antigen presenting cells are administered to a patient in an amount sufficient to elicit an effective CTL response to the virus or tumor antigen and/or to alleviate, reduce, cure or at least partially arrest symptoms and/or complications from the disease or infection. An amount adequate to accomplish this is defined as a “therapeutically effective dose.” The dose will be determined by the activity of dendritic cell produced and the condition of the patient, as well as the body weight or surface area of the patient to be treated. The size of the dose also will be determined by the existence, nature, and extent of any adverse side-effects that accompany the administration of a particular cell in a particular patient. In determining the effective amount of the cell to be administered in the treatment or prophylaxis of diseases such as cancer (e.g., metastatic melanoma, prostate cancer, etc.), the physician needs to evaluate circulating plasma levels. CTL toxicity, progression of the disease, and the induction of immune response against any introduced cell type

In a particular aspect, methods are provided for the treatment of various cancers and hyperproliferative diseases. Treatment methods will involve treating an individual with an effective amount of induced senescent cells. An effective amount is described, generally, as that amount sufficient to detectably and repeatedly to ameliorate, reduce, minimize or limit the extent of the disease or its symptoms, including its resistance to one or more therapies. More rigorous definitions may apply, including elimination, eradication or cure of a therapy-resistant disease.

To kill cells, inhibit cell growth, inhibit metastasis, decrease tumor or tissue size and otherwise reverse or reduce the malignant phenotype of cancer or tumor cells, using the methods and compositions described herein, one would generally administer induced senescent cells. This may be combined with compositions comprising other agents effective in the treatment of cancer, tumors or hyperproliferative cells or therapy-resistant cancer, tumors or hyperproliferative cells. These compositions would be provided in a combined amount effective to induce an immune response that can kill or inhibit proliferation of the a cancer cell. This process may involve administering to a subject the combination agent(s) or factor(s) at the same time. This may be achieved by administering to a subject a single composition or pharmacological formulation that includes both agents, or by administering to a subject two distinct compositions or formulations, at the same time, wherein one composition includes the induced senescent cells and the other includes the second agent.

Alternatively, the induced senescent cell therapy may precede or follow the other agent treatment by intervals ranging from minutes to weeks. In embodiments where the other agent and induced senescent cell therapy are applied separately to the cell, one would generally ensure that a significant period of time did not expire between the time of each delivery, such that the agent and induced senescent cell therapy would still be able to exert an advantageously combined effect on the subject. In such instances, it is contemplated that one may contact the subject or individual with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, such as the exemplary case wherein the induced senescent cell is “A” and the other therapy is “B”:

Other combinations particularly contemplated are: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of the induced senescent cells to a patient will follow general protocols for the administration of biotherapeutics. It is expected that the treatment cycles would be repeated as necessary. It also is contemplated that various standard therapies, as well as surgical intervention, may be applied in combination with the described induced senescent cells. A tumor, cancer cell mass or hyperproliferative cell focii may be surgically resected along with, prior to or subsequent to induced senescent cell administration.

Aqueous compositions comprise an effective amount of a compound, dissolved or dispersed in a pharmaceutically acceptable carrier or aqueous medium. Such compositions can also be referred to as inocula. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

In some embodiments, patients will have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl) for administration of a combined cancer therapy.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate or any analog or derivative variant thereof.

In some embodiments, chemotherapy is involved. For example, a subject may be or a subject may become resistant to one or more particular chemotherapies, and/or a chemotherapy may be employed in conjunction with a method such as administration of induced senescent cells. The term “chemotherapy” refers to the use of drugs to treat cancer. A “chemotherapeutic agent” is used to connote a compound or composition that is administered in the treatment of cancer. In certain aspects, a chemotharapeutic agent may also be used to induce senescence in a cancer cell or target cell that is later administered to a subject.

These chemotherapeutic agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM 1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor, vaccines such as gene therapy vaccines and pharmaceutically acceptable salts, acids or derivatives of any of the above.

In certain embodiments a chemotherapeutic agent may be selected from a list of FDA-approved oncology drugs with approved indications and date of approval, which may be obtained on the world wide web address of the U.S. Food and Drug Administration. Such chemotherapeutic agents or oncology drugs include those listed in Table 1.

TABLE 1

| | | | |
|---|---|---|---|
| Aldesleukin | Proleukin | | Chiron Corp |
| Alemtuzumab | Campath | Accel. Approv. (clinical benefit not | Millennium |
| | | established) Campath is indicated for | and ILEX |
| | | the treatment of B-cell chronic | Partners, LP |
| | | lymphocytic leukemia (B-CLL) in | |
| | | patients who have been treated with | |
| | | alkylating agents and who have failed | |
| | | fludarabine therapy. | |
| alitretinoin | Panretin | Topical treatment of cutaneous lesions | Ligand |
| | | in patients with AIDS-related Kaposi's | Pharmaceuticals |
| | | sarcoma. | |
| allopurinol | Zyloprim | Patients with leukemia, lymphoma and | GlaxoSmithKline |
| | | solid tumor malignancies who are | |
| | | receiving cancer therapy which causes | |
| | | elevations of serum and urinary uric | |
| | | acid levels and who cannot tolerate | |
| | | oral therapy. | |
| altretamine | Hexalen | Single agent palliative treatment of | US |
| | | patients with persistent or recurrent | Bioscience |
| | | ovarian cancer following first-line | |
| | | therapy with a cisplatin and/or | |
| | | alkylating agent based combination. | |
| amifostine | Ethyol | To reduce the cumulative renal toxicity | US |
| | | associated with repeated | Bioscience |
| | | administration of cisplatin in patients | |
| | | with advanced ovarian cancer | |
| amifostine | Ethyol | Accel. Approv. (clinical benefit not | US |
| | | established) Reduction of platinum | Bioscience |
| | | toxicity in non-small cell lung cancer | |
| amifostine | Ethyol | To reduce post-radiation xerostomia | US |
| | | for head and neck cancer where the | Bioscience |
| | | radiation port includes a substantial | |
| | | portion of the parotid glands. | |
| anastrozole | Arimidex | Accel. Approv. (clinical benefit not | AstraZeneca |
| | | established) for the adjuvant treatment | |
| | | of postmenopausal women with | |
| | | hormone receptor positive early breast | |
| | | cancer | |
| anastrozole | Arimidex | Treatment of advanced breast cancer | AstraZeneca |
| | | in postmenopausal women with | Pharmaceuticals |
| | | disease progression following | |
| | | tamoxifen therapy. | |
| anastrozole | Arimidex | For first-line treatment of | AstraZeneca |
| | | postmenopausal women with hormone | Pharmaceuticals |
| | | receptor positive or hormone receptor | |
| | | unknown locally advanced or | |
| | | metastatic breast cancer. | |
| arsenic | Trisenox | Second line treatment of relapsed or | Cell |
| trioxide | | refractory APL following ATRA plus an | Therapeutic |
| | | anthracycline. | |
| Asparaginase | Elspar | ELSPAR is indicated in the therapy of | Merck & Co, |
| | | patients with acute lymphocytic | Inc. |
| | | leukemia. This agent is useful primarily | |
| | | in combination with other | |
| | | chemotherapeutic agents in the | |
| | | induction of remissions of the disease | |
| | | in pediatric patients. | |
| BCG Live | TICE BCG | | Organon |
| | | | Teknika Corp |
| bexarotene | Targretin | For the treatment by oral capsule of | Ligand |
| capsules | | cutaneous manifestations of cutaneous | Pharmaceuticals |
| | | T-cell lymphoma in patients who are | |
| | | refractory to at least one prior systemic | |
| | | therapy. | |
| bexarotene | Targretin | For the topical treatment of cutaneous | Ligand |
| gel | | manifestations of cutaneous T-cell | Pharmaceuticals |
| | | lymphoma in patients who are | |
| | | refractory to at least one prior systemic | |
| | | therapy. | |
| bleomycin | Blenoxane | | Bristol-Myers |
| | | | Squibb |
| bleomycin | Blenoxane | Sclerosing agent for the treatment of | Bristol-Myers |
| | | malignant pleural effusion (MPE) and | Squibb |
| | | prevention of recurrent pleural | |
| | | effusions. | |
| busulfan | Busulfex | Use in combination with | Orphan |
| intravenous | | cyclophoshamide as conditioning | Medical, Inc. |
| | | regimen prior to allogeneic | |
| | | hematopoietic progenitor cell | |
| | | transplantation for chronic | |
| | | myelogenous leukemia. | |
| busulfan oral | Myleran | Chronic Myelogenous Leukemia- | GlaxoSmithKline |
| | | palliative therapy | |
| calusterone | Methosarb | | Pharmacia & |
| | | | Upjohn |
| | | | Company |
| capecitabine | Xeloda | Accel. Approv. (clinical benefit | Roche |
| | | subsequently established) Treatment | |
| | | of metastatic breast cancer resistant to | |
| | | both paclitaxel and an anthracycline | |
| | | containing chemotherapy regimen or | |
| | | resistant to paclitaxel and for whom | |
| | | further anthracycline therapy may be | |
| | | contraindicated, e.g., patients who | |
| | | have received cumulative doses of 400 | |
| | | mg/m2 of doxorubicin or doxorubicin | |
| | | equivalents | |
| capecitabine | Xeloda | Initial therapy of patients with | Roche |
| | | metastatic colorectal carcinoma when | |
| | | treatment with fluoropyrimidine therapy | |
| | | alone is preferred. Combination | |
| | | chemotherapy has shown a survival | |
| | | benefit compared to 5-FU/LV alone. A | |
| | | survival benefit over 5_FU/LV has not | |
| | | been demonstrated with Xeloda | |
| | | monotherapy. | |
| capecitabine | Xeloda | Treatment in combination with | Roche |
| | | docetaxel of patients with metastatic | |
| | | breast cancer after failure of prior | |
| | | anthracycline containing chemotherapy | |
| carboplatin | Paraplatin | Palliative treatment of patients with | Bristol-Myers |
| | | ovarian carcinoma recurrent after prior | Squibb |
| | | chemotherapy, including patients who | |
| | | have been previously treated with | |
| | | cisplatin. | |
| carboplatin | Paraplatin | Initial chemotherapy of advanced | Bristol-Myers |
| | | ovarian carcinoma in combination with | Squibb |
| | | other approved chemotherapeutic | |
| | | agents. | |
| carmustine | BCNU, BiCNU | | Bristol-Myers |
| | | | Squibb |
| carmustine with | Gliadel Wafer | For use in addition to surgery to | Guilford |
| Polifeprosan 20 | | prolong survival in patients with | Pharmaceuticals |
| Implant | | recurrent glioblastoma multiforme who | Inc. |
| | | qualify for surgery. | |
| celecoxib | Celebrex | Accel. Approv. (clinical benefit not | Searle |
| | | established) Reduction of polyp | |
| | | number in patients with the rare | |
| | | genetic disorder of familial | |
| | | adenomatous polyposis. | |
| chlorambucil | Leukeran | Chronic Lymphocytic Leukemia- | GlaxoSmithKline |
| | | palliative therapy | |
| chlorambucil | Leukeran | | GlaxoSmithKline |
| cisplatin | Platinol | Metastatic testicular-in established | Bristol-Myers |
| | | combination therapy with other | Squibb |
| | | approved chemotherapeutic agents in | |
| | | patients with metastatic testicular | |
| | | tumors who have already received | |
| | | appropriate surgical and/or | |
| | | radiotherapeutic procedures. An | |
| | | established combination therapy | |
| | | consists of Platinol, Blenoxane and | |
| | | Velbam. | |
| cisplatin | Platinol | Metastatic ovarian tumors - in | Bristol-Myers |
| | | established combination therapy with | Squibb |
| | | other approved chemotherapeutic | |
| | | agents: Ovarian-in established | |
| | | combination therapy with other | |
| | | approved chemotherapeutic agents in | |
| | | patients with metastatic ovarian tumors | |
| | | who have already received appropriate | |
| | | surgical and/or radiotherapeutic | |
| | | procedures. An established | |
| | | combination consists of Platinol and | |
| | | Adriamycin. Platinol, as a single agent, | |
| | | is indicated as secondary therapy in | |
| | | patients with metastatic ovarian tumors | |
| | | refractory to standard chemotherapy | |
| | | who have not previously received | |
| | | Platinol therapy. | |
| cisplatin | Platinol | as a single agent for patients with | Bristol-Myers |
| | | transitional cell bladder cancer which is | Squibb |
| | | no longer amenable to local treatments | |
| | | such as surgery and/or radiotherapy. | |
| cladribine | Leustatin, 2- | Treatment of active hairy cell leukemia. | R. W. Johnson |
| | CdA | | Pharmaceutical |
| | | | Research |
| | | | Institute |
| cyclophosphamide | Cytoxan, | | Bristol-Myers |
| | Neosar | | Squibb |
| cyclophosphamide | Cytoxan | | Bristol-Myers |
| | Injection | | Squibb |
| cyclophosphamide | Cytoxan | | Bristol-Myers |
| | Injection | | Squibb |
| cyclophosphamide | Cytoxan | | Bristol-Myers |
| | Tablet | | Squibb |
| cytarabine | Cytosar-U | | Pharmacia & |
| | | | Upjohn |
| | | | Company |
| cytarabine | DepoCyt | Accel. Approv. (clinical benefit not | Skye |
| liposomal | | established) Intrathecal therapy of | Pharmaceuticals |
| | | lymphomatous meningitis | |
| dacarbazine | DTIC-Dome | | Bayer |
| dactinomycin, | Cosmegen | | Merck |
| actinomycin D | | | |
| dactinomycin, | Cosmegan | | Merck |
| actinomycin D | | | |
| Darbepoetin | Aranesp | Treatment of anemia associated with | Amgen, Inc. |
| alfa | | chronic renal failure. | |
| Darbepoetin | Aranesp | Aranesp is indicated for the treatment | Amgen, Inc. |
| alfa | | of anemia in patients with non-myeloid | |
| | | malignancies where anemia is due to | |
| | | the effect of concomitantly | |
| | | administered chemotherapy. | |
| daunorubicin | DanuoXome | First line cytotoxic therapy for | Nexstar, Inc. |
| liposomal | | advanced, HIV related Kaposi's | |
| | | sarcoma. | |
| daunorubicin, | Daunorubicin | Leukemia/myelogenous/monocytic/ | Bedford Labs |
| daunomycin | | erythroid of adults/remission induction in | |
| | | acute lymphocytic leukemia of children | |
| | | and adults. | |
| daunorubicin, | Cerubidine | In combination with approved | Wyeth Ayerst |
| daunomycin | | anticancer drugs for induction of | |
| | | remission in adult ALL. | |
| Denileukin | Ontak | Accel. Approv. (clinical benefit not | Seragen, Inc. |
| diftitox | | established) treatment of patients with | |
| | | persistent or recurrent cutaneous T- | |
| | | cell lymphoma whose malignant cells | |
| | | express the CD25 component of the | |
| | | IL-2 receptor | |
| dexrazoxane | Zinecard | Accel. Approv. (clinical benefit | Pharmacia & |
| | | subsequently established) Prevention | Upjohn |
| | | of cardiomyopathy associated with | Company |
| | | doxorubicin administration | |
| dexrazoxane | Zinecard | reducing the incidence and severity of | Pharmacia & |
| | | cardiomyopathy associated with | Upjohn |
| | | doxorubicin administration in women | Company |
| | | with metastatic breast cancer who | |
| | | have received a cumulative | |
| | | doxorubicin dose of 300 mg/m2 and | |
| | | who will continue to receive | |
| | | doxorubicin therapy to maintain tumor | |
| | | control. It is not recommended for use | |
| | | with the initiation of doxorubicin | |
| | | therapy. | |
| docetaxel | Taxotere | Accel. Approv. (clinical benefit | Aventis |
| | | subsequently established) Treatment | Pharmaceutical |
| | | of patients with locally advanced or | |
| | | metastatic breast cancer who have | |
| | | progressed during anthracycline-based | |
| | | therapy or have relapsed during | |
| | | anthracycline-based adjuvant therapy. | |
| docetaxel | Taxotere | For the treatment of locally advanced | Aventis |
| | | or metastatic breast cancer which has | Pharmaceutical |
| | | progressed during anthracycline-based | |
| | | treatment or relapsed during | |
| | | anthracycline-based adjuvant therapy. | |
| docetaxel | Taxotere | For locally advanced or metastatic | Aventis |
| | | non-small cell lung cancer after failure | Pharmaceutical |
| | | of prior platinum-based chemotherapy. | |
| docetaxel | Taxotere | | Aventis |
| | | | Pharmaceutical |
| docetaxel | Taxotere | in combination with cisplatin for the | Aventis |
| | | treatment of patients with | Pharmaceutical |
| | | unresectable, locally advanced or | |
| | | metastatic non-small cell lung cancer | |
| | | who have not previously received | |
| | | chemotherapy for this condition. | |
| doxorubicin | Adriamycin, | | Pharmacia & |
| | Rubex | | Upjohn |
| | | | Company |
| doxorubicin | Adriamycin | Antibiotic, antitumor agent. | Pharmacia & |
| | PFS Injection- | | Upjohn |
| | intravenous | | Company |
| | injection | | |
| doxorubicin | Doxil | Accel. Approv. (clinical benefit not | Sequus |
| liposomal | | established) Treatment of AIDS-related | Pharmaceuticals, |
| | | Kaposi's sarcoma in patients with | Inc. |
| | | disease that has progressed on prior | |
| | | combination chemotherapy or in | |
| | | patients who are intolerant to such | |
| | | therapy. | |
| doxorubicin | Doxil | Accel. Approv. (clinical benefit not | Sequus |
| liposomal | | established) Treatment of metastatic | Pharmaceuticals, |
| | | carcinoma of the ovary in patient with | Inc. |
| | | disease that is refractory to both | |
| | | paclitaxel and platinum based | |
| | | regimens | |
| DROMOSTANOLONE | DROMO- | | Eli Lilly |
| PROPIONATE | STANOLONE | | |
| DROMOSTANOLONE | MASTERONE | | SYNTEX |
| PROPIONATE | INJECTION | | |
| Elliott's B | Elliott's B | Diluent for the intrathecal | Orphan |
| Solution | Solution | administration of methotrexate sodium | Medical, Inc. |
| | | and cytarabine for the prevention or | |
| | | treatment of meningeal leukemia or | |
| | | lymphocytic lymphoma. | |
| epirubicin | Ellence | A component of adjuvant therapy in | Pharmacia & |
| | | patients with evidence of axillary node | Upjohn |
| | | tumor involvement following resection | Company |
| | | of primary breast cancer. | |
| Epoetin alfa | epogen | EPOGENB is indicated for the | Amgen, Inc. |
| | | treatment of anemia related to therapy | |
| | | with zidovudine in HIV- infected | |
| | | patients. EPOGENB is indicated to | |
| | | elevate or maintain the red blood cell | |
| | | level (as manifested by the hematocrit | |
| | | or hemoglobin determinations) and to | |
| | | decrease the need for transfusions in | |
| | | these patients. EPOGEND is not | |
| | | indicated for the treatment of anemia in | |
| | | HIV-infected patients due to other | |
| | | factors such as iron or folate | |
| | | deficiencies, hemolysis or | |
| | | gastrointestinal bleeding, which should | |
| | | be managed appropriately. | |
| Epoetin alfa | epogen | EPOGENB is indicated for the | Amgen, Inc. |
| | | treatment of anemic patients | |
| | | (hemoglobin >10 to _<13 g/dL) | |
| | | scheduled to undergo elective, | |
| | | noncardiac, nonvascular surgery to | |
| | | reduce the need for allogeneic blood | |
| | | transfusions. | |
| Epoetin alfa | epogen | EPOGENB is indicated for the | Amgen, Inc. |
| | | treatment of anemia in patients with | |
| | | non-myeloid malignancies where | |
| | | anemia is due to the effect of | |
| | | concomitantly administered | |
| | | chemotherapy. EPOGEND is indicated | |
| | | to decrease the need for transfusions | |
| | | in patients who will be receiving | |
| | | concomitant chemotherapy for a | |
| | | minimum of 2 months. EPOGENB is | |
| | | not indicated for the treatment of | |
| | | anemia in cancer patients due to other | |
| | | factors such as iron or folate | |
| | | deficiencies, hemolysis or | |
| | | gastrointestinal bleeding, which should | |
| | | be managed appropriately. | |
| Epoetin alfa | epogen | EPOGEN is indicated for the treatment | Amgen, Inch |
| | | of anemia associated with CRF, | |
| | | including patients on dialysis (ESRD) | |
| | | and patients not on dialysis. | |
| estramustine | Emcyt | palliation of prostate cancer | Pharmacia & |
| | | | Upjohn |
| | | | Company |
| etoposide | Etopophos | Management of refractory testicular | Bristol-Myers |
| phosphate | | tumors, in combination with other | Squibb |
| | | approved chemotherapeutic agents. | |
| etoposide | Etopophos | Management of small cell lung cancer, | Bristol-Myers |
| phosphate | | first-line, in combination with other | Squibb |
| | | approved chemotherapeutic agents. | |
| etoposide | Etopophos | Management of refractory testicular | Bristol-Myers |
| phosphate | | tumors and small cell lung cancer. | Squibb |
| etoposide, | Vepesid | Refractory testicular tumors-in | Bristol-Myers |
| VP-16 | | combination therapy with other | Squibb |
| | | approved chemotherapeutic agents in | |
| | | patients with refractory testicular | |
| | | tumors who have already received | |
| | | appropriate surgical, chemotherapeutic | |
| | | and radiotherapeutic therapy. | |
| etoposide, | VePesid | In combination with other approved | Bristol-Myers |
| VP-16 | | chemotherapeutic agents as first line | Squibb |
| | | treatment in patients with small cell | |
| | | lung cancer. | |
| etoposide, | Vepesid | In combination with other approved | Bristol-Myers |
| VP-16 | | chemotherapeutic agents as first line | Squibb |
| | | treatment in patients with small cell | |
| | | lung cancer. | |
| exemestane | Aromasin | Treatment of advance breast cancer in | Pharmacia & |
| | | postmenopausal women whose | Upjohn |
| | | disease has progressed following | Company |
| | | tamoxifen therapy. | |
| Filgrastim | Neupogen | | Amgen, Inc. |
| Filgrastim | Neupogen | NEUPOGEN is indicated to reduce the | Amgen, Inc. |
| | | duration of neutropenia and | |
| | | neutropenia-related clinical sequelae, | |
| | | eg, febrile neutropenia, in patients with | |
| | | nonmyeloid malignancies undergoing | |
| | | myeloablative chemotherapy followed | |
| | | by marrow transplantation. | |
| Filgrastim | Neupogen | NEUPOGEN is indicated to decrease | Amgen, Inc. |
| | | the incidence of infection, as | |
| | | manifested by febrile neutropenia, in | |
| | | patients with nonmyeloid malignancies | |
| | | receiving myelosuppressive anticancer | |
| | | drugs associated with a significant | |
| | | incidence of severe neutropenia with | |
| | | fever. | |
| Filgrastim | Neupogen | NEUPOGEN is indicated for reducing | Amgen, Inc. |
| | | the time to neutrophil recovery and the | |
| | | duration of fever, following induction or | |
| | | consolidation hemotherapy treatment | |
| | | of adults with AML. | |
| floxuridine | FUDR | | Roche |
| (intraarterial) | | | |
| fludarabine | Fludara | Palliative treatment of patients with B- | Berlex |
| | | cell lymphocytic leukemia (CLL) who | Laboratories |
| | | have not responded or have | Inc. |
| | | progressed during treatment with at | |
| | | least one standard alkylating agent | |
| | | containing regimen. | |
| fluorouracil, | Adrucil | prolong survival in combination with | ICN Puerto |
| 5-FU | | leucovorin | Rico |
| fulvestrant | Faslodex | the treatment of hormone receptor- | IPR |
| | | positive metastatic breast cancer in | |
| | | postmenopausal women with disease | |
| | | progression following antiestrogen | |
| | | therapy | |
| gemcitabine | Gemzar | Treatment of patients with locally | Eli Lilly |
| | | advanced (nonresectable stage II or | |
| | | III) or metastatic (stage IV) | |
| | | adenocarcinoma of the pancreas. | |
| | | Indicated for first-line treatment and for | |
| | | patients previously treated with a 5- | |
| | | fluorouracil-containing regimen. | |
| gemcitabine | Gemzar | For use in combination with cisplatin | Eli Lilly |
| | | for the first-line treatment of patients | |
| | | with inoperable, locally advanced | |
| | | (Stage IIIA or IIIB) or metastatic (Stage | |
| | | IV) non-small cell lung cancer. | |
| gemtuzumab | Mylotarg | Accel. Approv. (clinical benefit not | Wyeth Ayerst |
| ozogamicin | | established) Treatment of CD33 | |
| | | positive acute myeloid leukemia in | |
| | | patients in first relapse who are 60 | |
| | | years of age or older and who are not | |
| | | considered candidates for cytotoxic | |
| | | chemotherapy. | |
| goserelin | Zoladex | Palliative treatment of advanced breast | AstraZeneca |
| acetate | Implant | cancer in pre- and perimenopausal | Pharmaceuticals |
| | | women. | |
| goserelin | Zoladex | | AstraZeneca |
| acetate | | | Pharmaceuticals |
| hydroxyurea | Hydrea | | Bristol-Myers |
| | | | Squibb |
| hydroxyurea | Hydrea | Decrease need for transfusions in | Bristol-Myers |
| | | sickle cell anemia | Squibb |
| Ibritumomab | Zevalin | Accel. Approv. (clinical benefit not | IDEC |
| Tiuxetan | | established) treatment of patients with | Pharmaceuticals |
| | | relapsed or refractory low-grade, | Corp |
| | | follicular, or transformed B-cell non- | |
| | | Hodgkin's lymphoma, including | |
| | | patients with Rituximab refractory | |
| | | follicular non-Hodgkin's lymphoma. | |
| idarubicin | Idamycin | For use in combination with other | Adria |
| | | approved antileukemic drugs for the | Laboratories |
| | | treatment of acute myeloid leukemia | |
| | | (AML) in adults. | |
| idarubicin | Idamycin | In combination with other approved | Pharmacia & |
| | | antileukemic drugs for the treatment of | Upjohn |
| | | acute non-lymphocytic leukemia in | Company |
| | | adults. | |
| ifosfamide | IFEX | Third line chemotherapy of germ cell | Bristol-Myers |
| | | testicular cancer when used in | Squibb |
| | | combination with certain other | |
| | | approved antineoplastic agents. | |
| imatinib | Gleevec | Accel. Approv. (clinical benefit not | Novartis |
| mesylate | | established) Initial therapy of chronic | |
| | | myelogenous leukemia | |
| imatinib | Gleevec | Accel. Approv. (clinical benefit not | Novartis |
| mesylate | | established) metastatic or | |
| | | unresectable malignant gastrointestinal | |
| | | stromal tumors | |
| imatinib | Gleevec | Accel. Approv. (clinical benefit not | Novartis |
| mesylate | | established) Initial treatment of newly | |
| | | diagnosed Ph+ chronic myelogenous | |
| | | leukemia (CML). | |
| Interferon | Roferon-A | | Hoffmann-La |
| alfa-2a | | | Roche Inc. |
| Interferon | Intron A | Interferon alfa-2b, recombinant for | Schering |
| alfa-2b | | injection is indicated as adjuvant to | Corp |
| | | surgical treatment in patients 18 years | |
| | | of age or older with malignant | |
| | | melanoma who are free of disease but | |
| | | at high risk for systemic recurrence | |
| | | within 56 days of surgery. | |
| Interferon | Intron A | Interferon alfa-2b, recombinant for | Schering |
| alfa-2b | | Injection is indicated for the initial | Corp |
| | | treatment of clinically aggressive | |
| | | follicular Non-Hodgkin's Lymphoma in | |
| | | conjunction with anthracycline- | |
| | | containing combination chemotherapy | |
| | | in patients 18 years of age or older. | |
| Interferon | Intron A | Interferon alfa-2b, recombinant for | Schering |
| alfa-2b | | Injection is indicated for intralesional | Corp |
| | | treatment of selected patients 18 years | |
| | | of age or older with condylomata | |
| | | acuminata involving external surfaces | |
| | | of the genital and perianal areas. | |
| Interferon | Intron A | Interferon alfa-2b, recombinant for | Schering |
| alfa-2b | | Injection is indicated for the treatment | Corp |
| | | of chronic hepatitis C in patients 18 | |
| | | years of age or older with | |
| | | compensated liver disease who have a | |
| | | history of blood or blood-product | |
| | | exposure and/or are HCV antibody | |
| | | positive. | |
| Interferon | Intron A | Interferon alfa-2b, recombinant for | Schering |
| alfa-2b | | Injection is indicated for the treatment | Corp |
| | | of chronic hepatitis B in patients 18 | |
| | | years of age or older with | |
| | | compensated liver disease and HBV | |
| | | replication. | |
| Interferon | Intron A | Interferon alfa-2b, recombinant for | Schering |
| alfa-2b | | Injection is indicated for the treatment | Corp |
| | | of patients 18 years of age or older | |
| | | with hairy cell leukemia. | |
| Interferon | Intron A | Interferon alfa-2b, recombinant for | Schering |
| alfa-2b | | Injection is indicated for the treatment | Corp |
| | | of selected patients 18 years of age or | |
| | | older with AIDS-Related Kaposi's | |
| | | Sarcoma. The likelihood of response to | |
| | | INTRON A therapy is greater in | |
| | | patients who are without systemic | |
| | | symptoms, who have limited | |
| | | lymphadenopathy and who have a | |
| | | relatively intact immune system as | |
| | | indicated by total CD4 count. | |
| Interferon | Intron A | | Schering |
| alfa-2b | | | Corp |
| Interferon | Intron A | | Schering |
| alfa-2b | | | Corp |
| Interferon | Intron A | | Schering |
| alfa-2b | Intron A | | Corp |
| irinotecan | Camptosar | Accel. Approv. (clinical benefit | Pharmacia & |
| | | subsequently established) Treatment | Upjohn |
| | | of patients with metastatic carcinoma | Company |
| | | of the colon or rectum whose disease | |
| | | has recurred or progressed following | |
| | | 5-FU-based therapy. | |
| irinotecan | Camptosar | Follow up of treatment of metastatic | Pharmacia & |
| | | carcinoma of the colon or rectum | Upjohn |
| | | whose disease has recurred or | Company |
| | | progressed following 5-FU-based | |
| | | therapy. | |
| irinotecan | Camptosar | For first line treatment n combination | Pharmacia & |
| | | with 5-FU/leucovorin of metastatic | Upjohn |
| | | carcinoma of the colon or rectum. | Company |
| letrozole | Femara | Treatment of advanced breast cancer | Novartis |
| | | in postmenopausal women. | |
| letrozole | Femara | First-line treatment of postmenopausal | Novartis |
| | | women with hormone receptor positive | |
| | | or hormone receptor unknown locally | |
| | | advanced or metastatic breast cancer. | |
| letrozole | Femara | | Novartis |
| leucovorin | Wellcovorin, | Leucovorin calcium is indicated fro use | Immunex |
| | Leucovorin | in combination with 5-fluorouracil to | Corporation |
| | | prolong survival in the palliative | |
| | | treatment of patients with advanced | |
| | | colorectal cancer. | |
| leucovorin | Leucovorin | | Immunex |
| | | | Corporation |
| leucovorin | Leucovorin | | Immunex |
| | | | Corporation |
| leucovorin | Leucovorin | | Immunex |
| | | | Corporation |
| leucovorin | Leucovorin | In combination with fluorouracil to | Lederle |
| | | prolong survival in the palliative | Laboratories |
| | | treatment of patients with advanced | |
| | | colorectal cancer. | |
| levamisole | Ergamisol | Adjuvant treatment in combination with | Janssen |
| | | 5-fluorouracil after surgical resection in | Research |
| | | patients with Dukes' Stage C colon | Foundation |
| | | cancer. | |
| lomustine, | CeeBU | | Bristol-Myers |
| CCNU | | | Squibb |
| meclorethamine, | Mustargen | | Merck |
| nitrogen mustard | | | |
| megestrol | Megace | | Bristol-Myers |
| acetate | | | Squibb |
| melphalan, | Alkeran | | GlaxoSmithKline |
| L-PAM | | | |
| melphalan, | Alkeran | Systemic administration for palliative | GlaxoSmithKline |
| L-PAM | | treatment of patients with multiple | |
| | | myeloma for whom oral therapy is not | |
| | | appropriate. | |
| mercaptopurine, | Purinethol | | GlaxoSmithKline |
| 6-MP | | | |
| mesna | Mesnex | Prevention of ifosfamide-induced | Asta Medica |
| | | hemorrhagic cystitis | |
| methotrexate | Methotrexate | | Lederle |
| | | | Laboratories |
| methotrexate | Methotrexate | | Lederle |
| | | | Laboratories |
| methotrexate | Methotrexate | | Lederle |
| | | | Laboratories |
| methotrexate | Methotrexate | | Lederle |
| | | | Laboratories |
| methotrexate | Methotrexate | osteosarcoma | Lederle |
| | | | Laboratories |
| methotrexate | Methotrexate | | Lederle |
| | | | Laboratories |
| methoxsalen | Uvadex | For the use of UVADEX with the UVAR | Therakos |
| | | Photopheresis System in the palliative | |
| | | treatment of the skin manifestations of | |
| | | cutaneous T-cell lymphoma (CTCL) | |
| | | that is unresponsive to other forms of | |
| | | treatment. | |
| mitomycin C | Mutamycin | | Bristol-Myers |
| | | | Squibb |
| mitomycin C | Mitozytrex | therapy of disseminated | Supergen |
| | | adenocarcinoma of the stomach or | |
| | | pancreas in proven combinations with | |
| | | other approved chemotherapeutic | |
| | | agents and as palliative treatment | |
| | | when other modalities have failed. | |
| mitotane | Lysodren | | Bristol-Myers |
| | | | Squibb |
| mitoxantrone | Novantrone | For use in combination with | Immunex |
| | | corticosteroids as initial chemotherapy | Corporation |
| | | for the treatment of patients with pain | |
| | | related to advanced hormone- | |
| | | refractory prostate cancer. | |
| mitoxantrone | Novantrone | For use with other approved drugs in | Lederle |
| | | the initial therapy for acute | Laboratories |
| | | nonlymphocytic leukemia (ANLL) in | |
| | | adults. | |
| nandrolone | Durabolin-50 | | Organon |
| phenpropionate | | | |
| Nofetumomab | Verluma | | Boehringer |
| | | | Ingelheim |
| | | | Pharma KG |
| | | | (formerly Dr. |
| | | | Karl Thomae |
| | | | GmbH) |
| Oprelvekin | Neumega | | Genetics |
| | | | Institute, Inc. |
| Oprelvekin | Neumega | | Genetics |
| | | | Institute, Inc. |
| Oprelvekin | Neumega | Neumega is indicated for the | Genetics |
| | | prevention of severe thrombocytopenia | Institute, Inc. |
| | | and the reduction of the need for | |
| | | platelet transfusions following | |
| | | myelosuppressive chemotherapy in | |
| | | adult patients with nonmyeloid | |
| | | malignancies who are at high risk of | |
| | | severe thrombocytopenia. | |
| oxaliplatin | Eloxatin | Accel. Approv. (clinical benefit not | Sanofi |
| | | established) in combination with | Synthelabo |
| | | infusional 5-FU/LV, is indicated for the | |
| | | treatment of patients with metastatic | |
| | | carcinoma of the colon or rectum | |
| | | whose disease has recurred or | |
| | | progressed during or within 6 months | |
| | | of completion of first line therapy with | |
| | | the combination of bolus 5-FU/LV and | |
| | | irinotecan. | |
| paclitaxel | Paxene | treatment of advanced AIDS-related | Baker Norton |
| | | Kaposi's sarcoma after failure of first | Pharmaceuticals, |
| | | line or subsequent systemic | Inc. |
| | | chemotherapy | |
| paclitaxel | Taxol | Treatment of patients with metastatic | Bristol-Myers |
| | | carcinoma of the ovary after failure of | Squibb |
| | | first-line or subsequent chemotherapy. | |
| paclitaxel | Taxol | Treatment of breast cancer after failure | Bristol-Myers |
| | | of combination chemotherapy for | Squibb |
| | | metastatic disease or relapse within 6 | |
| | | months of adjuvant chemotherapy. | |
| | | Prior therapy should have included an | |
| | | anthracycline unless clinically | |
| | | contraindicated. | |
| paclitaxel | Taxol | New dosing regimen for patients who | Bristol-Myers |
| | | have failed initial or subsequent | Squibb |
| | | chemotherapy for metastatic | |
| | | carcinoma of the ovary | |
| paclitaxel | Taxol | second line therapy for AIDS related | Bristol-Myers |
| | | Kaposi's sarcoma. | Squibb |
| paclitaxel | Taxol | For first-line therapy for the treatment | Bristol-Myers |
| | | of advanced carcinoma of the ovary in | Squibb |
| | | combination with cisplatin. | |
| paclitaxel | Taxol | for use in combination with cisplatin, | Bristol-Myers |
| | | for the first-line treatment of non-small | Squibb |
| | | cell lung cancer in patients who are not | |
| | | candidates for potentially curative | |
| | | surgery and/or radiation therapy. | |
| paclitaxel | Taxol | For the adjuvant treatment of node- | Bristol-Myers |
| | | positive breast cancer administered | Squibb |
| | | sequentially to standard doxorubicin- | |
| | | containing combination therapy. | |
| paclitaxel | Taxol | First line ovarian cancer with 3 hour | Bristol-Myers |
| | | infusion. | Squibb |
| pamidronate | Aredia | Treatment of osteolytic bone | Novartis |
| | | metastases of breast cancer in | |
| | | conjunction with standard | |
| | | antineoplastic therapy. | |
| pegademase | Adagen | Enzyme replacement therapy for | Enzon |
| | (Pegademase | patients with severe combined | |
| | Bovine) | immunodeficiency asa result of | |
| | | adenosine deaminase deficiency. | |
| Pegaspargase | Oncaspar | | Enzon, Inc. |
| Pegfilgrastim | Neulasta | Neulasta is indicated to decrease the | Amgen, Inc. |
| | | incidence of infection, as manifested | |
| | | by febrile neutropenia, in patients with | |
| | | non-myeloid malignancies receiving | |
| | | myelosuppressive anti-cancer drugs | |
| | | associated with a clinically significant | |
| | | incidence of febrile neutropenia. | |
| pentostatin | Nipent | Single agent treatment for adult | Parke-Davis |
| | | patients with alpha interferon refractory | Pharmaceutical |
| | | hairy cell leukemia. | Co. |
| pentostatin | Nipent | Single-agent treatment for untreated | Parke-Davis |
| | | hairy cell leukemia patients with active | Pharmaceutical |
| | | disease as defined by clinically | Co. |
| | | significant anemia, neutropenia, | |
| | | thrombocytopenia, or disease-related | |
| | | symptoms. (Supplement for front - | |
| | | line therapy.) | |
| pipobroman | Vercyte | | Abbott Labs |
| plicamycin, | Mithracin | | Pfizer Labs |
| mithramycin | | | |
| porfimer | Photofrin | For use in photodynamic therapy | QLT |
| sodium | | (PDT) for palliation of patients with | Phototherapeutics |
| | | completely obstructing esophageal | Inc. |
| | | cancer, or patients with partially | |
| | | obstructing esophageal cancer who | |
| | | cannot be satisfactorily treated with | |
| | | ND-YAG laser therapy. | |
| porfimer | Photofrin | For use in photodynamic therapy for | QLT |
| sodium | | treatment of microinvasive | Phototherapeutics |
| | | endobronchial nonsmall cell lung | Inc. |
| | | cancer in patients for whom surgery | |
| | | and radiotherapy are not indicated. | |
| porfimer | Photofrin | For use in photodynamic therapy | QLT |
| sodium | | (PDT) for reduction of obstruction and | Phototherapeutics |
| | | palliation of symptoms in patients with | Inc. |
| | | completely or partially obstructing | |
| | | endobroncial nonsmall cell lung cancer | |
| | | (NSCLC). | |
| procarbazine | Matulane | | Sigma Tau |
| | | | Pharms |
| quinacrine | Atabrine | | Abbott Labs |
| Rasburicase | Elitek | ELITEK is indicated for the initial | Sanofi- |
| | | management of plasma uric acid levels | Synthelabo, |
| | | in pediatric patients with leukemia, | Inc. |
| | | lymphoma, and solid tumor | |
| | | malignancies who are receiving anti- | |
| | | cancer therapy expected to result in | |
| | | tumor lysis and subsequent elevation | |
| | | of plasma uric acid. | |
| Rituximab | Rituxan | | Genentech, |
| | | | Inc. |
| Sargramostim | Prokine | | Immunex |
| | | | Corp |
| streptozocin | Zanosar | Antineoplastic agent. | Pharmacia & |
| | | | Upjohn |
| | | | Company |
| talc | Sclerosol | For the prevention of the recurrence of | Bryan |
| | | malignant pleural effusion in | |
| | | symptomatic patients. | |
| tamoxifen | Nolvadex | | AstraZeneca |
| | | | Pharmaceuticals |
| tamoxifen | Nolvadex | As a single agent to delay breast | AstraZeneca |
| | | cancer recurrence following total | Pharmaceuticals |
| | | mastectomy and axillary dissection in | |
| | | postmenopausal women with breast | |
| | | cancer (T1-3, N1, M0) | |
| tamoxifen | Nolvadex | For use in premenopausal women with | AstraZeneca |
| | | metastatic breast cancer as an | Pharmaceuticals |
| | | alternative to oophorectomy or ovarian | |
| | | irradiation | |
| tamoxifen | Nolvadex | For use in women with axillary node- | AstraZeneca |
| | | negative breast cancer adjuvant | Pharmaceuticals |
| | | therapy. | |
| tamoxifen | Nolvadex | Metastatic breast cancer in men. | AstraZeneca |
| | | | Pharmaceuticals |
| tamoxifen | Nolvadex | Equal bioavailability of a 20 mg | AstraZeneca |
| | | Nolvadex tablet taken once a day to a | Pharmaceuticals |
| | | 10 mg Nolvadex tablet taken twice a | |
| | | day. | |
| tamoxifen | Nolvadex | to reduce the incidence of breast | AstraZeneca |
| | | cancer in women at high risk for breast | Pharmaceuticals |
| | | cancer | |
| tamoxifen | Nolvadex | In women with DCIS, following breast | AstraZeneca |
| | | surgery and radiation, Nolvadex is | Pharmaceuticals |
| | | indicated to reduce the risk of invasive | |
| | | breast cancer. | |
| temozolomide | Temodar | Accel. Approv. (clinical benefit not | Schering |
| | | established) Treatment of adult | |
| | | patients with refractory anaplastic | |
| | | astrocytoma, i.e., patients at first | |
| | | relapse with disease progression on a | |
| | | nitrosourea and procarbazine | |
| | | containing regimen | |
| teniposide, | Vumon | In combination with other approved | Bristol-Myers |
| VM-26 | | anticancer agents for induction therapy | Squibb |
| | | in patients with refractory childhood | |
| | | acute lymphoblastic leukemia (all). | |
| testolactone | Teslac | | Bristol-Myers |
| | | | Squibb |
| testolactone | Teslac | | Bristol-Myers |
| | | | Squibb |
| thioguanine, | Thioguanine | | GlaxoSmithKline |
| 6-TG | | | |
| thiotepa | Thioplex | | Immunex |
| | | | Corporation |
| thiotepa | Thioplex | | Immunex |
| | | | Corporation |
| thiotepa | Thioplex | | Lederle |
| | | | Laboratories |
| topotecan | Hycamtin | Treatment of patients with metastatic | GlaxoSmithKline |
| | | carcinoma of the ovary after failure of | |
| | | initial or subsequent chemotherapy. | |
| topotecan | Hycamtin | Treatment of small cell lung cancer | GlaxoSmithKline |
| | | sensitive disease after failure of first- | |
| | | line chemotherapy. In clinical studies | |
| | | submitted to support approval, | |
| | | sensitive disease was defined as | |
| | | disease responding to chemotherapy | |
| | | but subsequently progressing at least | |
| | | 60 days (in the phase 3 study) or at | |
| | | least 90 days (in the phase 2 studies) | |
| | | after chemotherapy | |
| toremifene | Fareston | Treatment of advanced breast cancer | Orion Corp. |
| | | in postmenopausal women. | |
| Tositumomab | Bexxar | Accel. Approv. (clinical benefit not | Corixa |
| | | established) Treatment of patients with | Corporation |
| | | CD20 positive, follicular, non-Hodgkin's | |
| | | lymphoma, with and without | |
| | | transformation, whose disease is | |
| | | refractory to Rituximab and has | |
| | | relapsed following chemotherapy | |
| Trastuzumab | Herceptin | HERCEPTIN as a single agent is | Genentech, |
| | | indicated for the treatment of patients | Inc. |
| | | with metastatic breast cancer whose | |
| | | tumors overexpress the HER2 protein | |
| | | and who have received one or more | |
| | | chemotherapy regimens for their | |
| | | metastatic disease. | |
| Trastuzumab | Herceptin | Herceptin in combination with | Genentech, |
| | | paclitaxel is indicated for treatment of | Inc. |
| | | patients with metastatic breast cancer | |
| | | whose tumors overexpress the HER-2 | |
| | | protein and had not received | |
| | | chemotherapy for their metastatic | |
| | | disease | |
| Trastuzumab | Herceptin | | Genentech, |
| | | | Inc. |
| Trastuzumab | Herceptin | | Genentech, |
| | | | Inc. |
| Trastuzumab | Herceptin | | Genentech, |
| | | | Inc. |
| tretinoin, | Vesanoid | Induction of remission in patients with | Roche |
| ATRA | | acute promyelocytic leukemia (APL) | |
| | | who are refractory to or unable to | |
| | | tolerate anthracycline based cytotoxic | |
| | | chemotherapeutic regimens. | |
| Uracil | Uracil Mustard | | Roberts Labs |
| Mustard | Capsules | | |
| valrubicin | Valstar | For intravesical therapy of BCG- | Anthra --> |
| | | refractory carcinoma in situ (CIS) of | Medeva |
| | | the urinary bladder in patients for | |
| | | whom immediate cystectomy would be | |
| | | associated with unacceptable | |
| | | morbidity or mortality. | |
| vinblastine | Velban | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vincristine | Oncovin | | Eli Lilly |
| vinorelbine | Navelbine | Single agent or in combination with | GlaxoSmithKline |
| | | cisplatin for the first-line treatment of | |
| | | ambulatory patients with unresectable, | |
| | | advanced non-small cell lung cancer | |
| | | (NSCLC). | |
| vinorelbine | Navelbine | Navelbine is indicated as a single | GlaxoSmithKline |
| | | agent or in combination with cisplatin | |
| | | for the first-line treatment of | |
| | | ambulatory patients with | |
| | | unreseactable, advanced non-small | |
| | | cell lung cancer (NSCLC). In patients | |
| | | with Stage IV NSCLC, Navelbine is | |
| | | indicated as a single agent or in | |
| | | combination with cisplatin. In Stage III | |
| | | NSCLC, Navelbine is indicated in | |
| | | combination with cisplatin. | |
| zoledronate | Zometa | the treatment of patients with multiple | Novartis |
| | | myeloma and patients with | |
| | | documented bone metastases from | |
| | | solid tumors, in conjunction with | |
| | | standard antineoplastic therapy. | |
| | | Prostate cancer should have | |
| | | progressed after treatment with at least | |
| | | one hormonal therapy | |

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which an induced senescent cells and a chemotherapeutic or radiotherapeutic agent are delivered to a subject. To achieve cell killing or stasis, both agents are delivered to a subject in a combined amount effective to kill the cancerous cells or prevent them from dividing.

It is noted that both radiation and chemotherapeutics can be used to induce senescence, and therefore, any discussion in the context of therapy may also be implemented in the context of inducing senescence in cells that then may be used in a therapy.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Adoptive Transfer of Immunity Using Senescent Cells to Activate a Subject's Own Bone Marrow Derived DCs.

Patient blood or bone marrow-dendritic cells (BM-DC) are generated by following a standard protocol (Meng 2010). BM cells are isolated from the blood and cultured in complete RPMI supplemented with 20 ng/ml hGM-CSF (R&D Systems) for 5-7 days. These DCs are co-cultured with senescent tumor cells established from individual patient, either in the transwell coculture systems, in closed contact or fused by the membrane destabilizing agent polyethylene glycol (PEG) or by electroporation. In the transwell system, DCs are stimulated to proliferate and mature in the cytokine cocktail provided by senescent tumor cells. In the attached coculture DCs may acquire broad tumor associated antigen (TAAs) by direct contact and phagocytosis while proliferating and maturing in the cytokine cocktail of senescent tumor cells. However, DCs-senescent tumor fusion vaccines may allow DCs to express the entire repertoire of TAAs of the fused tumor cell, and to process endogenously and present tumor epitopes via MHC class I and II pathways to activate both CD4⁺ and CD8⁺ T cells. After 5 days, the DCs are collected from coculture with veliparib+IR treated senescent tumor cells and infused into patients by intravascular, intraperitoneal, subcutaneousor other routes at levels of (e.g.) 5×10⁵ cells once a week for 3 weeks. Optionally, all vaccine compositions are utilized in combination with immunotherapies, such as IFNγ, IL-2, IL-12, GM-CSF or CpG.

Using Patient Peripheral Blood Mononuclear Cells (PBMCs) as Vaccine.

PBMCs are co-cultured with senescent tumor cells and/or in combination with IL-2 and/or IL-12. Each treatment cycle consists of 21 days and it includes administration of PBMCs alone, PBMCs and IL-2 or IL-12, or PBMCs and both IL-2 and IL-12. Injection with senescent-pulsed PBMCs and IL-12 takes place on day 1, followed by IL-12 injections alone on days 3 and 5, followed by a 16 day rest period. Optionally, this procedure is performed in combination with IL-2, IL-2 alone, or PBMCs alone.

Tumor Inoculation, Treatments.

Mouse melanoma cell line B16SIY (5×10⁵ cells) were implanted subcutaneously in the flank of 6 to 8-wk-old C57/B6 mice. Tumors were allowed to grow until they reached a volume of about 100 to 150 mm³ (approximately 2 wk) before treatment with PARP inhibitor veliparib (vrib, (R)-2-(2-methylpyrrolidin-2-yl)-1H-benzo[d]imidazole-4-carboxamide, ABT-888) and/or IR (Dunn 2006). Mice received 0.5 mg of veliparib in water twice daily by oral gavage as indicated. CD8⁺, CD4⁺, NK cells were depleted by anti-CD8 or anti-CD4 or anti-NK1.1, respectively, 1 d before IR. Depletion was confirmed by checking peripheral blood samples by flow cytometry. Macrophages were depleted with liposomal clodronate starting 1 d before IR. Depletion was confirmed by checking splenocyte and tumor samples by flow cytometry (Zhang 2008). Samples were collected 2 to 7 days later, and analyzed by flow cytometry using a BD LSRII flow cytometer. Blood samples were gated in forward scatter and side scatter on smaller cells for CD8, CD4 analysis, larger cells for NK and macrophage, and the percentage of different cell types were determined (Lugade 2008, Zhang 2010).

Histopathology, Immunohistochemistry and Real Time RT-PCR.

Slides were deparaffinized in xylene and hydrated with alcohol before being placed in 0.3% H₂O₂/methanol blocking solution to quench endogenous peroxidase activity followed by subsequent antigen unmasking in EDTA buffer. Incubation with the primary goat polyclonal biotinylated anti-mouse CCL2, CCL5, CXCL9, CXCL10 or IFNβ (dilution 1:5; R&D Systems), then the staining was revealed by using specific secondary antibodies conjugated to a horseradish peroxidase-labeled polymer or to an alkaline phosphatase-labeled polymer. Reactions were developed with 3,3′-diaminobenzidine chromogen or Vulcan Red, respectively, and counterstained with hematoxylin. Negative controls were obtained by using isotype-matched primary antibody IgG. The SA-βGal assay was performed using the Senescence Beta-galactosidase Staining Kit (Cell Signaling) (Dunn 2006). All images were captured using Zeiss Axiovert 200M and Zeiss Axiocam color digital camera controlled by OpenLab software with a 20× objective. All primers sequence and running protocols for RT-PCR can be found in reference Table 2 (Lugade 2008, Zhang 2008).

TABLE 2
Primer sequences

| | | SEQ | | SEQ |
|---|---|---|---|---|
| | | ID | | ID |
| GENE | FORWARD | NO | REVERSE | NO |
| CCL2 | ATTGGGATCATCTTG | 1 | CCTGCTGTTCACAGT | 2 |
| | CTGGT | | TGCC | |
| CCL3 | GTGGAATCTTCCGGC | 3 | ACCATGACACTCTGC | 4 |
| | TGTAG | | AACCA | |
| CCL4 | GAAACAGCAGGAAGT | 5 | CATGAAGCTCTGCGT | 6 |
| | GGGAG | | GTCTG | |
| CCL5 | CCACTTCTTCTCTGG | 7 | GTGCCCACGTCAAGG | 8 |
| | GTTGG | | AGTAT | |
| CXCL9 | TAGGCAGGTTTGATC | 9 | CGATCCACTACAAAT | 10 |
| | TCCGT | | CCCTCA | |
| CXCL10 | CCTATGGCCCTCATT | 11 | CTCATCCTGCTGGGT | 12 |
| | CTCAC | | CTGAG | |
| CXCL11 | CGCCCCTGTTTGAAC | 13 | CTGCTGAGATGAACA | 14 |
| | ATAAG | | GGAAGG | |
| IFNβ | CCCAGTGCTGGAGAA | 15 | CCCTATGGAGATGAC | 16 |
| | ATTGT | | GGAGA | |
| IFNγ | TGAGCTCATTGAATG | 17 | ACAGCAAGGCGAAAA | 18 |
| | CTTGG | | AGGAT | |
| TNFa | AGGGTCTGGGCCATA | 19 | CCACCACGCTCTTCT | 20 |
| | GAACT | | GTCTAC | |
| IL1α | CCAGAAGAAAATGAG | 21 | AGCGCTCAAGGAGAA | 22 |
| | GTCGG | | GACC | |
| IL1β | GGTCAAAGGTTTGGA | 23 | TGTGAAATGCCACCT | 24 |
| | AGCAG | | TTTGA | |
| IL6 | ACCAGAGGAAATTTT | 25 | TGATGCACTTGCAGA | 26 |
| | CAATAGGC | | AAACA | |
| CD8α | GCC CCG TGG CTC | 27 | CTG ACT AGC GGC | 28 |
| | AGT GAA GG | | CTG GGA CA | |
| CD8β | ACT TCT GCG CGA | 29 | TGG GGG AAC GGG | 30 |
| | CGG TTG GG | | CAT TGC TTC | |
| P16 | CGT GAA CAT GTT | 31 | CGA ATC TGC ACC | 32 |
| | GTT GAG GC | | GTA GTT GA | |
| P21 | CGG TGT CAG AGT | 33 | CGA AGT CAA AGT | 34 |
| | CTA GGG GA | | TCC ACC GT | |
| P27 | GCCAGGATGTCAGCG | 35 | AAGGCCGGGCTTCTT | 36 |
| | GGAGC | | GGGC | |
| P57 | GACCCGACTCCGGAC | 37 | AGTCGTTCGCATTGG | 38 |
| | CCGAT | | CCGCA | |
| GAPDH | AACGACCCCTTCATT | 39 | TCCACGACATACTCA | 40 |
| | GAC | | GCAC | |

BM-DC Generation, Selection of CD4+ T Cells, Coculture with Veliparib+IR or IR Alone Treated Tumors Cells.

B16SIY cells were maintained in a full medium supplemented with 10% FCS. The cells were pretreated with 10 μm veliparib then exposed to 6 or 12 Gy x-ray or IR alone (Gammacell 1000; MDS Nordion, Kanata, Ontario, Canada), then cocultured with immature BM-DC in a transwell system. 3 days later BM-DC were collected and analyzed by FACS for cell surface maturation marker and intracellular cytokines. These BM-DC were used to stimulate sorted CD4+ cells (Lugade 2008, Zhang 2008).

Senescent Cell Vaccine Preparation, Tumor Rechallenge or Adjuvant Therapy.

B16SIY cells were pretreated with veliparib/IR as above. 4 days later cell were collected and 5×10⁵ cells were injected subcutaneously at the right leg. The same number of live or irradiated B16SIY cells was injected as control vaccines. 5 days later, live B16SIY cells (5×10⁵) were injected at either the same site or the opposite leg. Tumor incidence and growth were counted and measured. Some mice received the 2nd live tumor cells injections 5 weeks later for rechallenge. Veliparib/IR pretreated B16 tumor cells were also sorted according to the cell size and granularity by FACS for pure senescent cells, then injected on the opposite leg of established B16 melanoma tumors in combination with local IR. Tumor growth was measured.

Detection and Isolation of Senescent Cells.

The procedure to enrich senescent cells from a heterogeneous population begins with procurement of cell samples. These samples may be obtained from mouse or human tissue samples, or cultured cell lines. Cells from other organisms of interest (e.g. rat; yeast) may also be used. Cells are briefly grown in culture and senescence is then induced by the addition of ABT-888 PARP inhibitor followed by IR. Alternatively, senescence may be induced by DNA damaging or oxidizing reagents, overexpression of senescence-inducing oncogenes, or by passage of cells in culture until the point of replicative senescence.

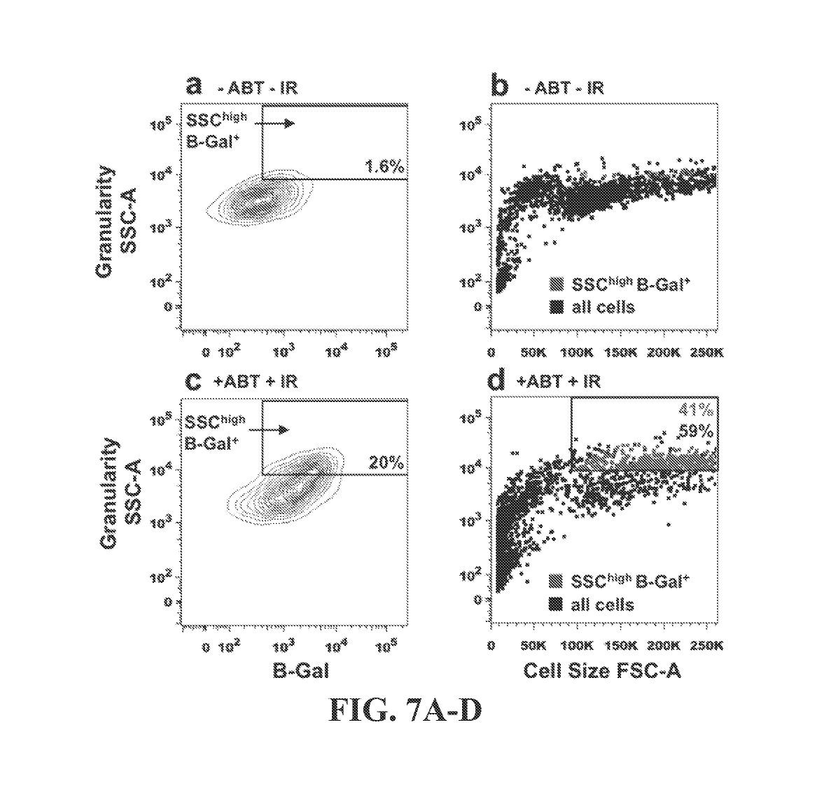

Following senescence induction, cells are collected and incubated briefly in the presence of Bafilomycin A1, a reagent known to selectively increase lysosomal pH to ˜6.0. At this pH, SA-βGal is optimally detected, while endogenous beta-galactosidase detection is minimized, reducing assay background. The fluorescent galactosidase substrate DDAO-Galactoside (DDAO-G) is then added to react with SA-βGal, emitting red fluorescence from senescent cells (FIG. 7).

The stained cells are then analyzed and sorted by flow cytometry (FACS). Cells may be sorted by FACS using DDAO (red) emission alone, DDAO vs. FSC (a proxy for size), or DDAO vs. SSC (a proxy for granularity). Using FSC or SSC as secondary sorting parameters affords an extra measure of certainty to senescent FACS, given that many, but not all, cell types show an increase in size, granularity, or both, upon induction of accelerated senescence.

Following cytometric cell sorting, the collected senescent cells can be used in a variety of assays; they can be re-introduced into culture, and stained with fluorescent probes for microscopy, and/or supernatant collected for analysis of secreted cytokines; they can be lysed, for protein or DNA analysis by electrophoresis and blotting; or, they can be injected as a vaccine into mice or humans, in which preliminary data has shown that injection of senescent cells stimulates anti-tumor activity of innate cytotoxic T-cells (CTLs).

Because cells in a heterogeneous population senesce to different extents depending on experimental conditions, populations of cells exposed to senescent accelerants rarely, if ever, approach 100% senescence. Most populations hover around 30% senescence after induction. A method to identify and enrich senescent cells to create 100% senescent, viable cell cultures using a readily available probe compatible with existing instrumentation is an extremely useful advance in methodology. The method described enables insight into mechanisms of senescence and the use of senescent cells as vaccine, which has previously been obscured by suboptimal assays and probes poorly suited to viable-cell FACS sorting.

To examine the effect of glucose metabolism on IRIF persistence in living cells, we exploited our previously described IRIF reporter consisting of GFP fused to the 53BP1 IRIF binding domain, expressed under tetracycline-inducible control. MCF7^{Tet-On} GFP-IBD cells were seeded at 3×10⁵ per Fluorodish (World Precision Instruments, Inc.) in high-glucose (4.5 g/L) DMEM (Invitrogen) media supplemented with 10% Tet system-approved fetal bovine serum (Clontech) and 1 μg/mL doxycycline (Sigma). Next day media was exchanged for either high-glucose (4.5 g/L) or low-glucose (1 g/L) DMEM with 10% FBS and 1 μg/mL doxycycline. 24 h later cells have been exposed for 6 Gy IR. Cells were fixed at 3 h and 24 h for IRIF imaging and at day 5 for SA-βGal staining. Images were captured on Zeiss Axiovert 200M and a Hammatsu Orca ER FireWire digital monochrome (IRIF) or Zeiss Axiocam color digital camera (SA-βGal staining) controlled by OpenLab software.

To investigate what step of glycolysis is critical for improvement of DNA repair we used set of well established glycolysis inhibitors including glucose transporter Glut1 inhibitor, HXKi, GAPHi, PKi, LDHi. Inhibition of any step of glycolysis resulted in IRIF persistence and induced senescence of MCF7 cells. Given that elevated glycolysis predispose tumor cells for therapeutic resistance similar experiment has been performed on IR resistant PANC02 mouse pancreatic and U87 human glioma cell lines.

PANC02^{Tet-On} GFP-IBD and U87^{Tet-On} GFP-IBD cell lines have been developed. Cells were seeded at 3×10⁵ per Fluorodish in media supplemented with 1 μg/mL doxycycline following pretreatment for 1 h with glycolysis inhibitors before 6 Gy irradiation at 48 h. Phloretin Glut1i—Glucose transporter 1 inhibitor 100 μM (Sigma), HXi—Hexokinase inhibitorII 50 μM (Calbiochem), PKi—Pyruvate kinase inhibitor oxalate 50 μM (Sigma), LDHi—Lactate dehydrogenase inhibitor, oxamate 1 mM (Sigma).

Head and neck squamous carcinomas cell line Nu61^{Tet-On} GFP-IBD with IRIF live imaging reporter have been developed. Female athymic nude mice underwent s.c. injection of 1×10⁷ Nu61 Tet-OnGFP-IBD cells in 100 μL of PBS. Nine days later, mice received 25 mg/kg veliparib (ChemieTek) by oral gavage 48 hours before and 72 hours after a single dose of 6 Gy. Mice were treated with 10 mg of 2-deoxy-D-glucose by intraperitoneal injection 5 days before and 5 days after IR. Mice were euthanized at day 5 after IR. Tumors were excised and frozen in liquid nitrogen for subsequent analysis. Frozen sections were analyzed for SA-βGal activity. The senescence-associated β-galactosidase (SA-βGal) assay was conducted as described before. Images were captured on a Zeiss Axiovert 200M and Zeiss Axiocam color digital camera controlled by OpenLab software with a ×20 objective.

In tumor exposed to irradiation alone, SA-βGal positive cells were not observed. Irradiation combined with glycolysis inhibitor 2DG induced numerous cells stained positive for SA-βGal, even more then irradiation combined with the PARP inhibitor veliparib (positive control). Strongest induction of SA-βGal we observed in irradiated tumor treated with 2DG and veliparib. We suggest that glycolysis inhibitors may cooperate with PARP inhibitors for irradiation induced senescence in IR-resistant tumor.

A Method for Flow Cytometric Identification and Enrichment of Viable Senescent Cells.

Prostate carcinoma (PCa) cell lines included 22Rv1 (human) and TRAMP-C2 (murine, ATCC). PCa cell lines were propagated in complete culture medium using sterile culture flasks in a humidified, 5% CO₂ incubator at 37° C. For 22Rv1 human PCa cells, complete media consisted of RPMI-1640 1× (modified without L-glutamine, Invitrogen) supplemented with fetal bovine serum (FBS, 10%, Gemini Biosciences), stabilized L-glutamine (2 mM, Gemini Biosciences), and penicillin-streptomycin solution (100 U/ml penicillin, 100 μg/ml streptomycin, Invitrogen). For TRAMP-C2 murine PCa cells, complete medium consisted of DMEM (modified with 4.5 g/l glucose and without L-glutamine, Invitrogen) supplemented with FBS (5%), Nu-Serum IV (5%, BD Biosciences), stabilized L-glutamine (4 mM), dehydroisoandrosterone (10 nM, Sigma-Aldrich), bovine insulin (5 μg/ml, Sigma-Aldrich), and penicillin-streptomycin solution (100 U/ml penicillin, 100 μg/ml streptomycin).

Cells were plated at low density (˜25×10³ cells/cm²) and incubated overnight to allow adherence to culture surfaces. The following day, PARP inhibitor veliparib (ABT-888, 10 μM in DMSO, ChemieTek) was added (or not) to freshly changed media to enhance uptake of the compound. Cells were incubated with veliparib for 60 minutes at 37° C. in 5% CO₂ prior to gamma irradiation (IR, 6 Gy). Treated and untreated cells were then incubated undisturbed for 5 days to allow senescence to proceed.

On day 5, cell monolayers were washed with Dulbecco's PBS (D-PBS, Ca2⁺ and Mg2⁺-free, Corning CellGro) and dissociated from culture surfaces via trypsin-EDTA (0.25% trypsin, 0.53 mM EDTA. Invitrogen) for 5 minutes at 37° C. followed by additional detachment using a sterile cell lifter to ensure collection of senescent cells, which were observed to have enhanced adherence. Cells were pelleted by centrifugation for 5 minutes at 1000×g, supernatant removed, and pellet resuspended in 1% bovine serum albumin (BSA, United States Biological) in DPBS. Cell suspensions were counted (cells/mL) using a handheld counting device (Scepter, Millipore). 500,000 cells were aliquoted per sample prior to beta-galactosidase (SA-βGal) staining via red fluorescent probe DDAO-Galactoside (DDAO-G).

In order to raise lysosomal pH to ˜6.0, the optimal pH at which to detect senescence-associated beta-galactosidase (SA-βGal), the known pH modulator Bafilomycin A1 (Baf, 100 nM, Sigma-Aldrich) was added to cell samples in 1 ml of 1% BSA-DPBS for 30 minutes. Baf incubation was carried out in a 37° C. dry incubator without CO₂. At t=30 min, DDAO-G (10 μg/ml, Invitrogen) was added directly to the Baf-modulated cell samples without an intermediate wash step. Cells were stained at 37° C. without CO₂ for 60 minutes, washed, and placed at 4° C. until analysis (<60 min).

Analysis of SA-βGal signal was conducted by flow cytometry, using an LSRII cytometer (Becton Dickinson) equipped with a 633 nm red diode laser and a 670/30 (APC) bandpass filter suitable for DDAO red fluorescent signal excitation and emission detection. 10,000 events were collected per sample and single-cell data exported as a listmode (.fcs) file to post-acquisition data analysis software (FlowJo, TreeStar). To define (gate) senescent populations, viable cell populations were visualized first on a scatter plot of FSC-A (size) vs SSC-A (granularity), and cellular debris was gated out. The whole-cell population was then visualized on a scatter plot of FSC-W vs SSC-W to discriminate doublets, which were then gated out of the analysis. Single, whole cells were then visualized on a red-fluorescence vs SSC-A plot in order to define senescent SA-βGal⁺ SSC^{high} cells. Untreated samples were used to set the senescent cell gating thresholds. The gated senescent cells were then backgated to show their distribution over total events.

### Example 2

### Results

Induction of Senescence and Inhibition of Tumor Growth by Veliparib and Radiation.

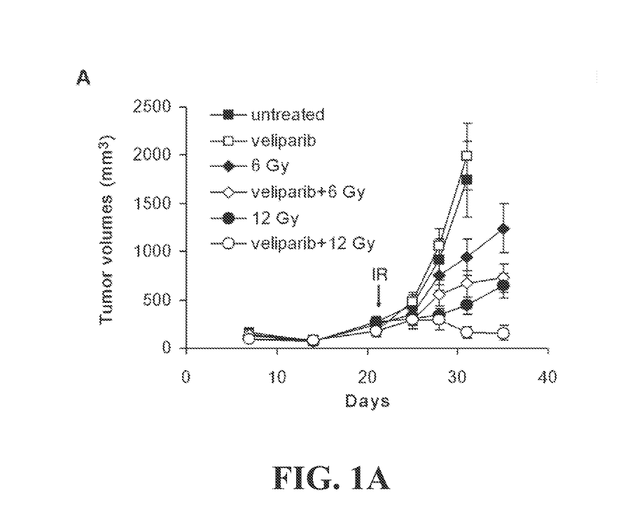

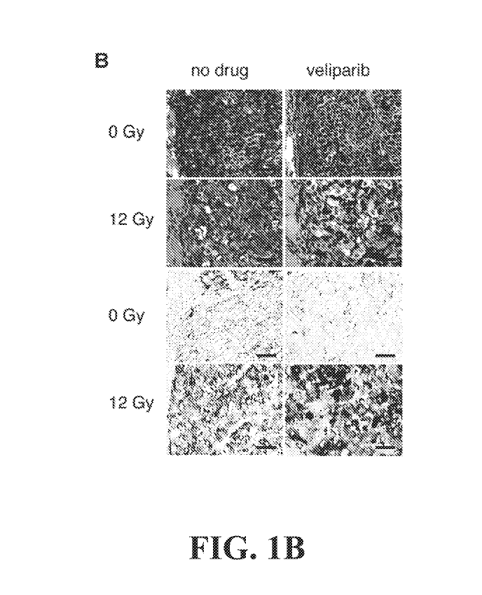

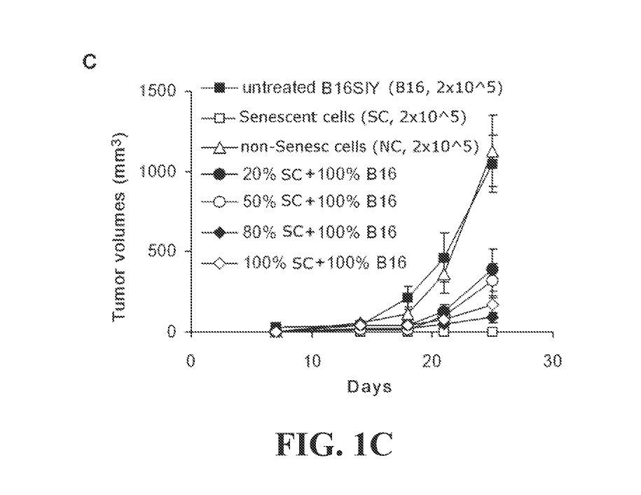

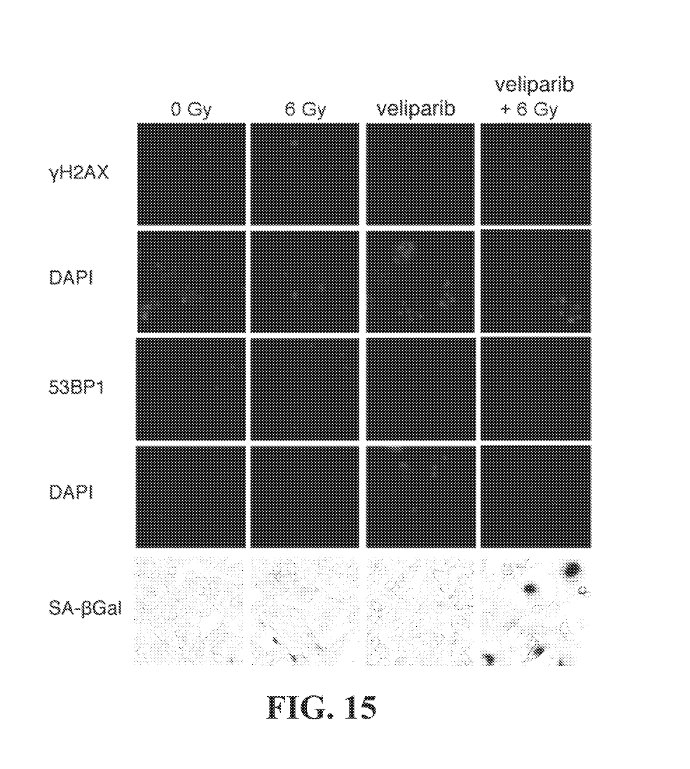

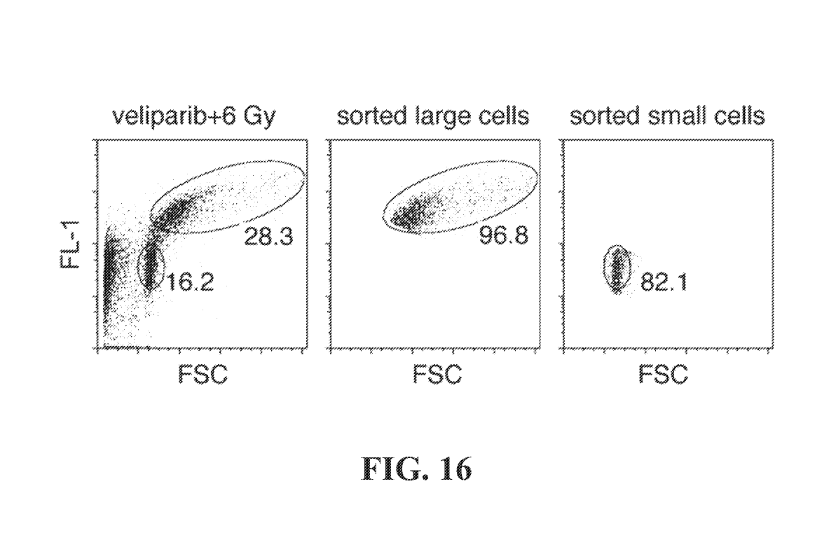

Our prior work combining PARPi with radiation (Efimova 2010, Barreto-Andrade 2011) was limited to analysis of human tumor cell lines in vitro and in xenograft tumors in immunodeficient athymic nude mice, where the role of the adaptive immune system cannot be examined. As a model, we used the mouse melanoma cell line B16SIY (Meng 2010, Lee 2009, Meng 2010), which grows rapidly after implantation in syngeneic C57/B6 mice to form radiation-resistant tumors. As with the human cell lines, treating B16 with ionizing radiation (IR) and the PARPi veliparib delayed DNA damage foci resolution marked by persistence of γH2AX and p53BP1 at 24 h, and induced accelerated senescence as shown by characteristic flattened cell morphology and enhanced senescence associated beta-galactosidase activity (SA-βGal) at day 7 (FIG. 15). While 6 or 12 Gy alone slowed tumor growth, combining IR with veliparib, 0.5 mg twice daily for 2 days prior to irradiation and then for 7 days thereafter (veliparib+IR), markedly delayed tumor regrowth (p=0.033, p=0.004, FIG. 1A). Examining the treated tumors for senescence revealed greater numbers of enlarged cells and more intense SA-βGal staining in the veliparib+IR treated tumors than those treated with IR or veliparib alone (FIG. 1B). One interpretation of these data is that the senescent B16 cells may be able to suppress recovery of surviving, non-senescent tumor cells. To test this, we treated B16 cells in vitro with veliparib+IR to induce accelerated senescence and then after 7 days, we sorted the surviving B16 cells to obtain populations of large, senescent cells and small, “non-senescent” cells, based on cell size and cell granularity (FIG. 16). The sorted senescent B16 cells failed to form tumors. However, the sorted small cells, like untreated B16 cells, readily formed growing tumors within two weeks (FIG. 1C). In turn, mixing sorted senescent cells with untreated B16 cells before coinjection into mice caused a marked tumor growth delay, suggesting that senescent cells can directly suppress proliferation of unirradiated B16 cells.

Altered Immuno-Regulatory Cytokine Components of the SASP in Irradiated Tumor Cells.

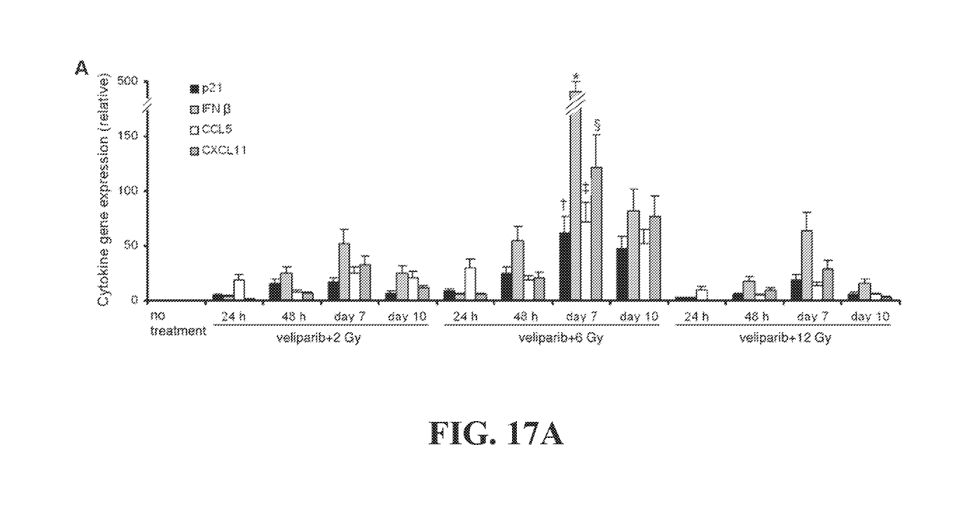

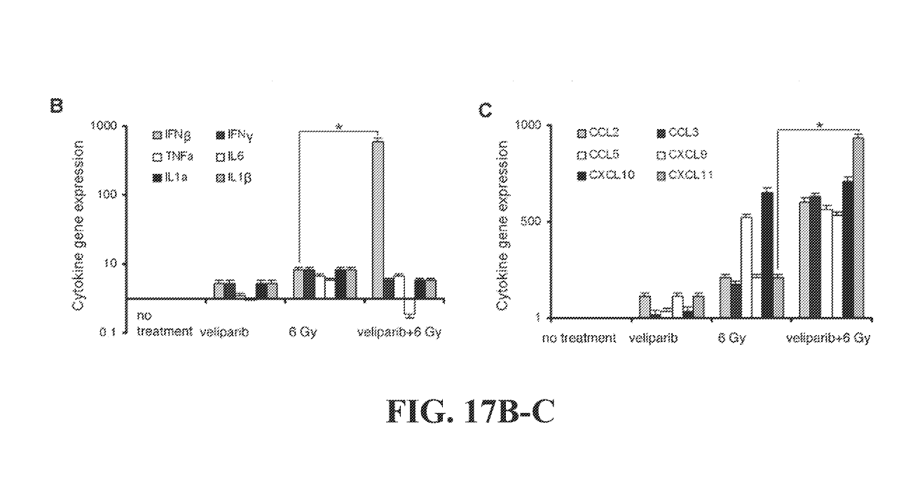

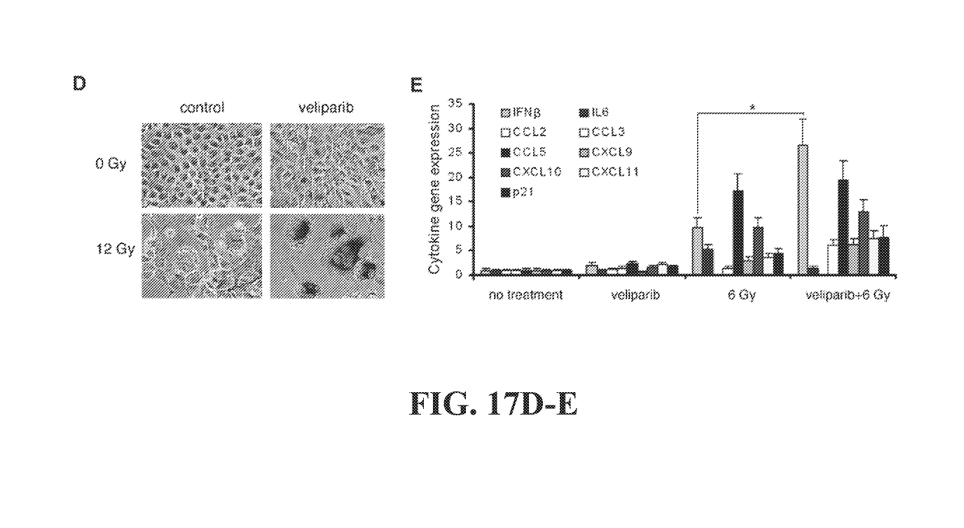

A simple model is that the senescent cells formed with valiparib and radiation were able to effect proliferation of other tumor cells via paracrine activity of the SASP. When B16 melanoma cells were treated in vitro with veliparib and/or 2 to 12 Gy IR and compared to untreated controls, RT-qPCR analysis demonstrated time- and dose-dependent changes in the radiation-induced secretome. We observe a shift in expression of multiple immuno-regulatory cytokines previously identified as part of the SASP (Rodier 2009, Orjalo 2009). The effect was greatest 7 days after a 6 Gy dose, when the B16 cells were strongly induced toward senescence, based on morphology and SA-βGal (FIG. 17A). The altered secretome displayed increased transcription of IFNβ (p=0.005) and decreased IL-6 (p=0.010) (FIG. 17B). Chemokines including monocyte-dendritic cell (DC), natural killer (NK) and CTL attractants CCL2, CCL3, CCL5, CXCL9, CXCL10, and especially CXCL11 expression (p=0.006) were also upregulated. (FIG. 17C). Similarly, treatment of the murine pancreatic cancer cell line p1048 induced SA-βGal staining and upregulation of multiple cytokines by 7 days after irradiation (FIG. 17D, 17E), suggesting a general effect of veliparib+IR.

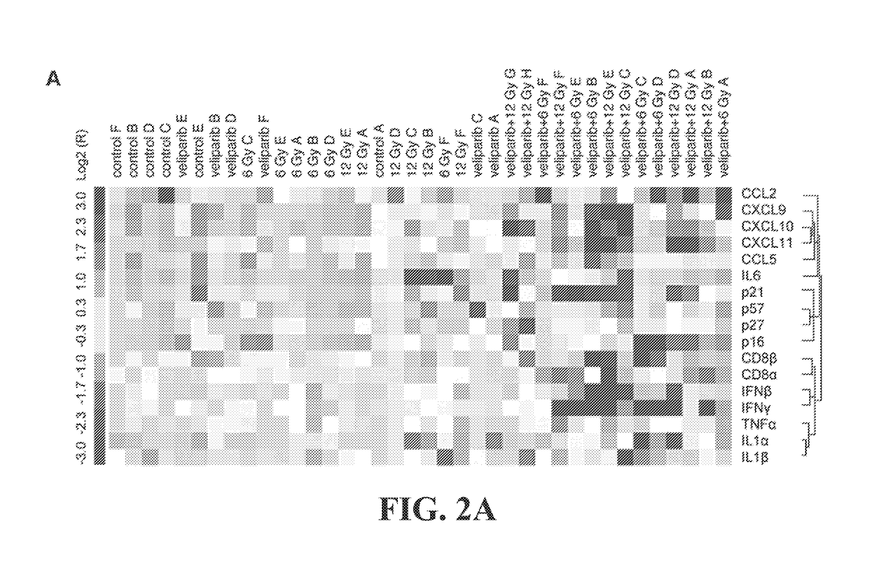

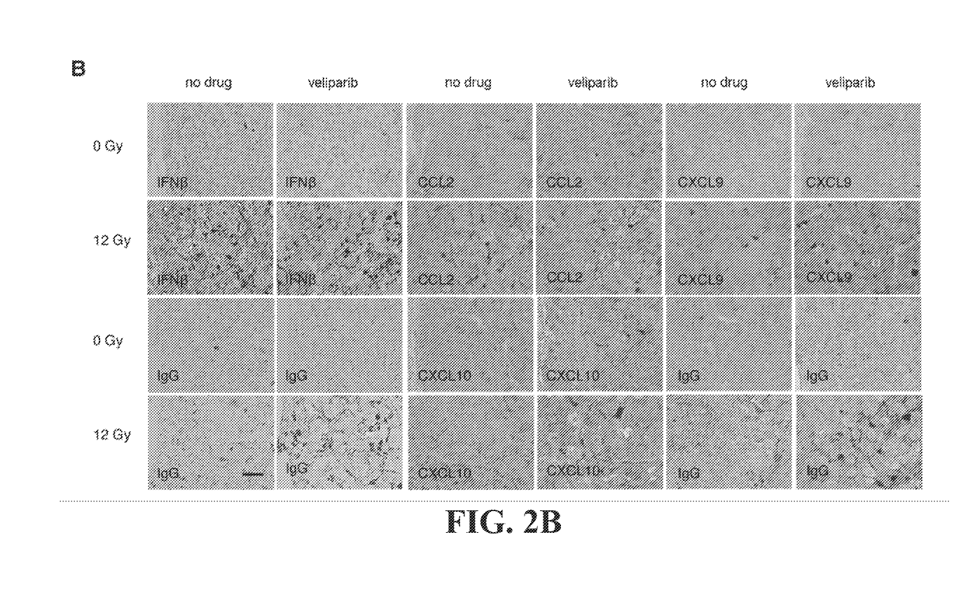

Thus, we investigated the influence of veliparib on gene expression in irradiated B16 tumors. RT-qPCR was performed on sets of lysates derived from tumors treated with veliparib and/or 0, 6 or 12 Gy to examine expression of cytokines and senescence markers. Expression was normalized to GAPDH and relative expression was compared and clustered using dChip software (FIG. 2A). Clustering revealed that most veliparib+IR-treated tumors displayed significantly increased expression of p21 and p16 compared to IR alone (p=0.024, p=0.021), consistent with enhanced accelerated senescence. Similarly, veliparib+IR increased expression of SASP genes including IFNβ (p=0.023), IFNγ (p=0.070), CCL2 (p=0.029), CCL5, CXCL9, CXCL10 and CXCL11 (p=0.004). Immunohistochemistry confirmed these changes, demonstrating a distinct strong staining of IFNβ, CCL2, CXCL9 and CXCL10 localized to the enlarged senescent tumor cells (FIG. 2B). Together, the changes to the SASP upon treatment with veliparib+IR appeared to skew expression toward immunostimulatory factors both in vitro and in vivo.

Activated Immune Response to Senescent Tumor Cells.

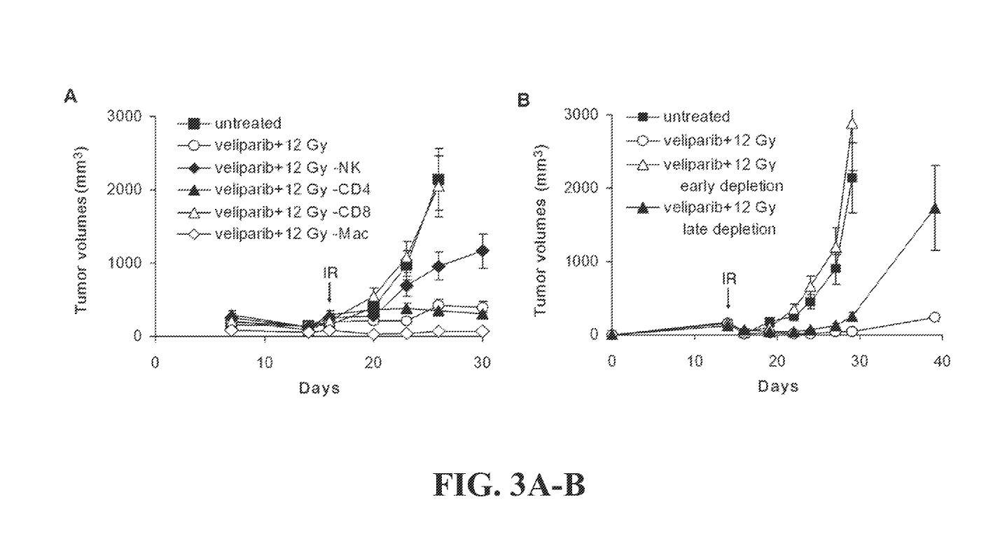

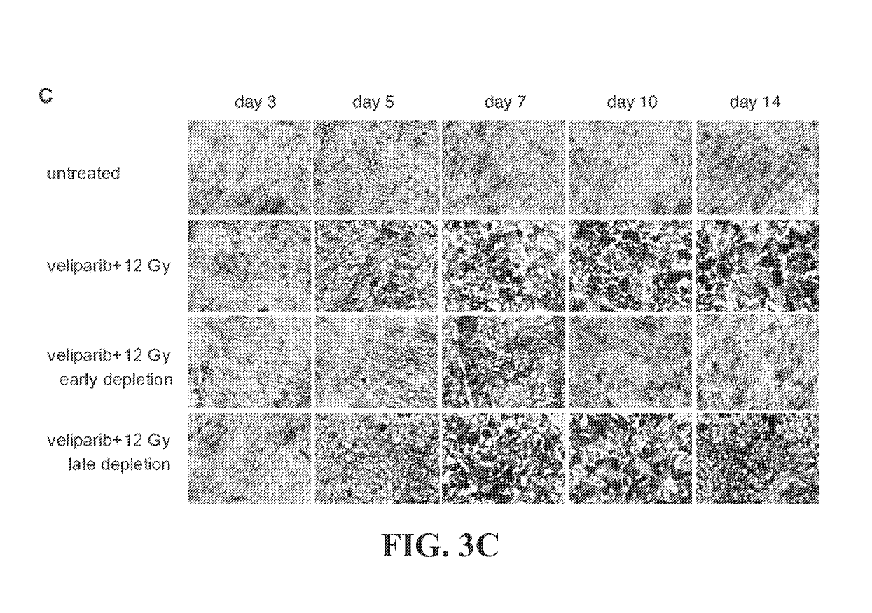

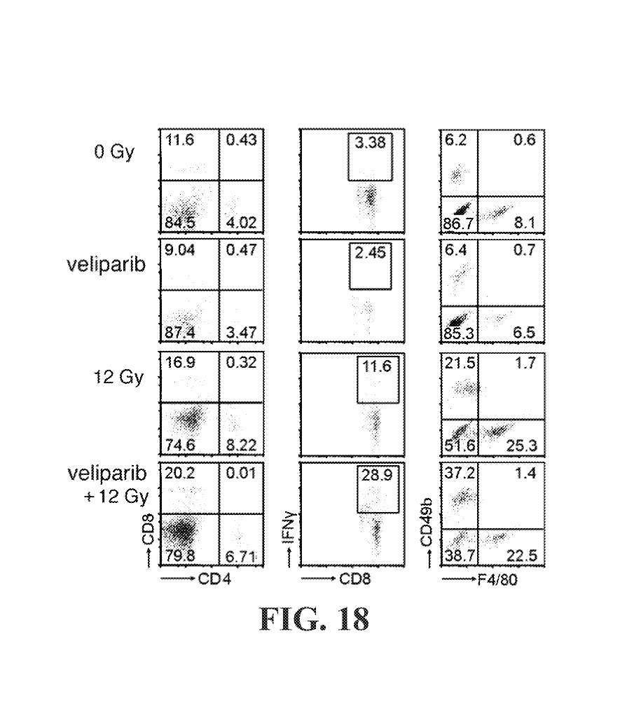

Based on our earlier observation that antigen-specific CD8+ CTLs partly mediate the benefits of radiation (Meng 2010, Lee 2009) and given the pattern of the SASP after veliparib+IR treatment, we were curious whether an anti-tumor immune response might contribute to the growth delay. Thus, before treating their tumors with veliparib+IR, we first treated tumor-bearing mice with antibodies to deplete CD4+ helper T cells, CD8+ cytotoxic T cells, or NK cells or with liposomal clodronate to deplete macrophages. Strikingly, the enhanced anti-tumor effect of veliparib+IR was abrogated by depleting CD8+ cells (p=0.003) and attenuated by loss of NK cells (p=0.009). Eliminating CD4+ cells had little effect on tumor regrowth after veliparib+IR (p=0.257) and depleting macrophages further delayed regrowth, consistent with prior studies of IR alone (Xue 2007, Rakhra 2010, Meng 2010) (FIG. 3A). Correspondingly, the proportion of IFNγ-producing CD8+ cells among tumor infiltrating lymphocytes was higher after treatment with veliparib+IR (29%) compared to IR alone (12%). NK cells were also more abundant in veliparib+IR treated tumors (FIG. 18). Taken together, these results support the hypothesis that accelerated senescence induced by veliparib+IR may exert its anti-tumor effect via an altered SASP that mediates recruitment and/or activation of CD8+ and/or NK cells.

To further characterize the role of CTLs, B16 tumor-bearing animals were injected daily with CD8+ cell depleting antibodies starting either 1 day before (early depletion) or 7 days after (late depletion) treatment with veliparib and 12 Gy. Early depletion of CD8+ cells eliminated the tumor growth delay induced by veliparib+IR (p=0.002 at day 29) while late depletion attenuated the effect (p=0.006 at day 39, FIG. 3B). Comparing tumor histology among treatments showed that early depletion decreased the accumulation or persistence of large, SA-βGal-staining senescent cells while late depletion appeared to rapidly reverse the histological changes (FIG. 3C).

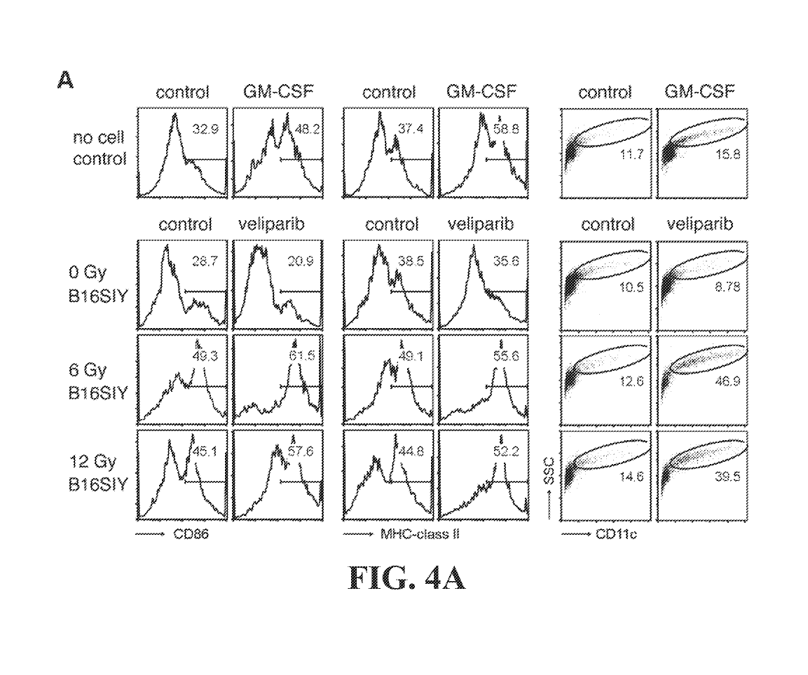

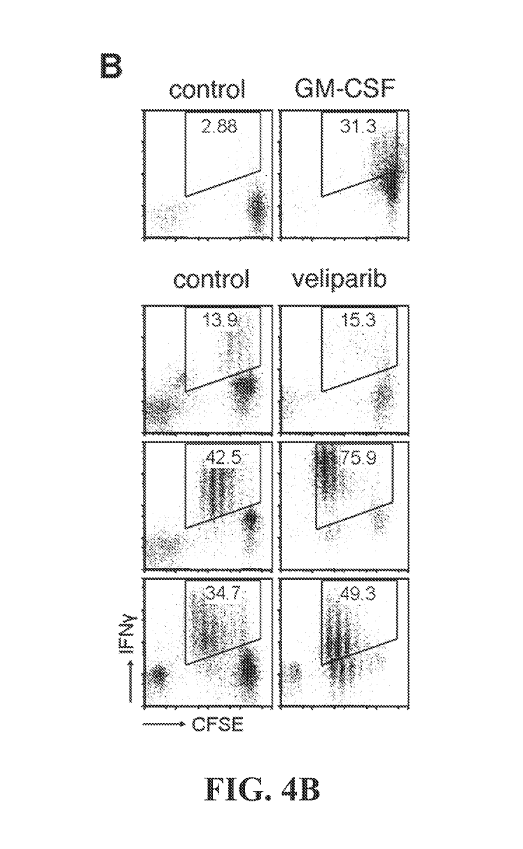

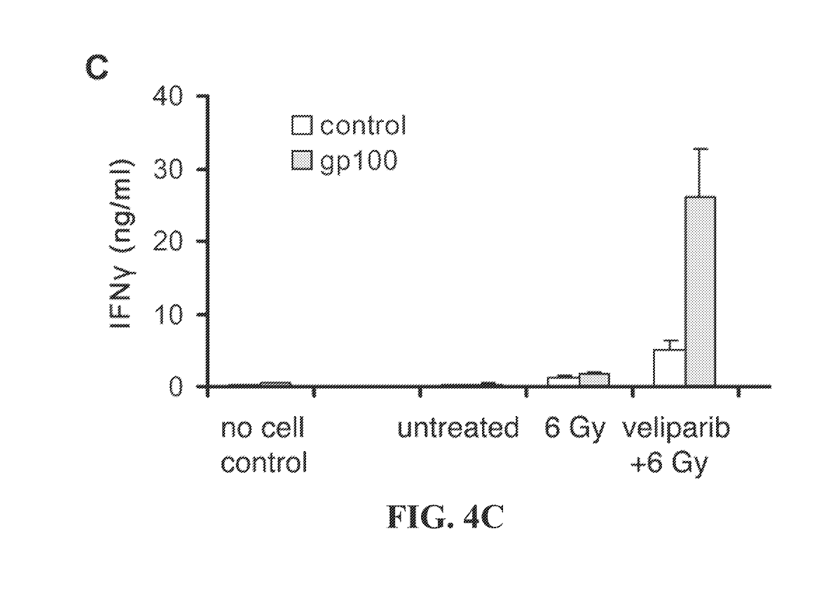

If accumulation of senescent B16 cells after veliparib+IR leads to activation of CD8+ cytotoxic T cells targeting the tumor, a likely mediator is enhanced function of antigen presenting cells (APCs). To detect signaling from senescent cells to APCs, immature bone marrow DCs were co-cultured with B16 tumor cells that had been treated with veliparib and/or 0, 6 or 12 Gy and compared to growth in GM-CSF containing medium as control. Flow cytometry demonstrated increased CD11c+ cell proliferation when cocultured with B16 cells treated with veliparib+6 Gy compared to either untreated or irradiated cells. Veliparib+IR-treated cells also enhanced CD11c+ cell maturation, as DCs displayed higher MHC-II+ and CD86+ fractions compared to coculture with cells treated only with radiation (FIG. 4A). In turn, the DCs collected from coculture with veliparib+IR treated B16 tumor cells better stimulated CD8+ cell proliferation, resulting in a higher level of IFNγ expression (FIG. 4B). These results point towards a mechanism in which DCs that enter a tumor following treatment with veliparib+IR may engulf antigens from senescent tumor cells and mature in a high IFNβ environment, providing more efficient priming of CD8+ cells to target the tumor, and driving enhanced proliferation and effector function in the draining lymph nodes (DLNs) and/or tumor microenvironment. Indeed, abundant tumor-specific T cells were present in the DLNs of mice received B16 tumor cells pretreated with veliparib+IR, as IFNγ secretion from DLN cells was readily detected upon stimulation with the B16 tumor antigen gp100 (FIG. 4C).

Senescent Cell Vaccine Blocks Tumor Formation.

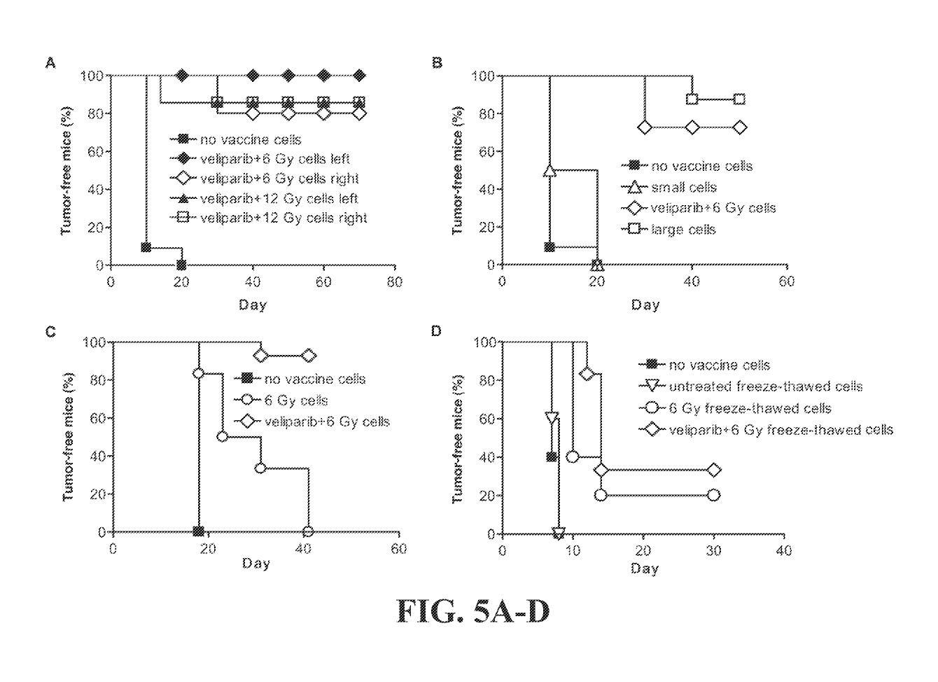

Together, these data raised the question whether veliparib+IR-induced senescent cells might be able to serve as a tumor cell vaccine and induce a CTL response sufficient to prevent tumor formation. As an initial test, we treated B16 cells with veliparib+IR in vitro and incubated for 7 days, and then injected them on the right leg of a mouse. Then, after 7 days, we injected untreated B16 cells on both the right and left legs as a challenge. While control mice all developed solid tumors on both legs within 2 weeks after challenge, over 80% of mice inoculated with senescent cells failed to grow tumors on either leg (FIG. 5A). To determine whether the senescent B16 cells provided the anti-tumor vaccine effect, we injected flow-sorted large senescent cells, small non-senescent cells, the unsorted cells as positive control, and untreated cells as negative control. After 7 days, the mice were challenged with B16 cells and tumor formation was followed (FIG. 5B). Only when senescent cells were injected was a vaccine effect observed, with the greatest effect from purified senescent cells.

These data raised the concern that the vaccine effect of senescent cells might be specific to the B16 murine melanoma model. Thus, we examined two other murine tumors, P1048 pancreatic adenocarcinoma (Stangl 2011) and TUBO breast adenocarcinoma (Masuelli 2007), which each overexpress either endogenous mouse Her2 or rat Her2/neu as a tumor antigen. When treated with veliparib and 12 Gy, while the P1048 cells respond by entering accelerated senescence and displaying an altered SASP (e.g. FIG. 17D, 17E), TUBO cells fail to display either the characteristic cell morphology or SA-βGal expression (data not shown). When injected into mice, P1048 cells treated with veliparib+IR induced Her2-specific IFNγ producing T cells in the DLNs (data not shown) and served as an effective vaccine against subsequent challenge with untreated P1048 cells (FIG. 5C). However, injection of TUBO cells treated with veliparib alone, IR alone or veliparib+IR failed to induce Her2-specific IFNγ producing T cells (data not shown) and provided little or no protection against tumor formation after challenge with untreated TUBO cells (FIG. 5D).

Potentiation of Radiation by Senescent Cells as Therapeutic Vaccine for Established Tumors.

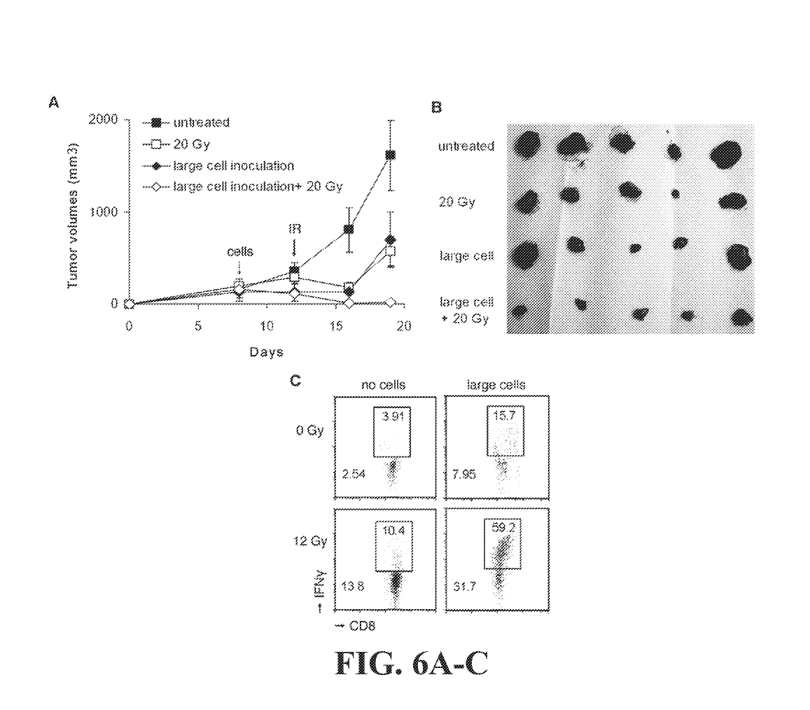

A remaining question was whether the veliparib+IR induced senescent tumor cells might be able to target established B16 tumors, serving as a therapeutic vaccine. Thus, mice were injected with untreated B16 cells on the right leg and after 7 days, when tumors could be readily detected, they were inoculated on the left leg with PBS or with sorted large, senescent cells prepared as above. After 5 days, tumors were treated with 0 or 20 Gy to evaluate the effect on regrowth after irradiation. Treating with IR alone or senescent tumor cells alone each delayed outgrowth significantly compared to the mock-treated control (p=0.016, p=0.038). However, combining inoculation with senescent cells at a remote site and local irradiation appeared to block outgrowth completely (p=0.003) (FIG. 6A, B). These data suggest the ability of senescent cells to enhance radiation, and implicate an adaptive immune response in the mechanism. Indeed, the proportion of tumor infiltrating CD8+ cells (32%) and the fraction of IFNγ-producing cells (59%) were markedly higher in tumors treated with senescent cells and IR compared to IR alone (14%, 10%) or senescent cells alone (8%, 16%, FIG. 6C). Considered together these results demonstrate that injection of senescent tumor cells can serve as a vaccine for radiation-inducible immunotherapy, suppressing tumor growth at distant sites through infiltration of IFNγ-producing CD8+ cells, targeted to the tumors by irradiation.

Senescent TUBO Cells Induced in Low Glucose Media Prevented Tumor Growth.

While injection of TUBO cells growing in high glucose medium after treatment with veliparib+IR failed to block tumor growth upon rechallenge with untreated TUBO cells, injection of veliparib+IR-treated TUBO cells growing in low glucose medium blocked tumor formation at higher rate (FIG. 11A), indicating the increased immunogenicity of TUBO cell prepared in low glucose medium where bioenergy restriction might cooperate with the DNA damage response. There was no increased cell apoptosis when comparing veliparib+IR-treated TUBO cells growing in low glucose medium to that growing in high glucose medium, surprisingly there were more cells survived IR and veliparib+IR in low glucose medium. More TUBO cells displayed enhanced senescence-associated β-galactosidase staining (SA-βGal) in low glucose media after treatment with veliparib+IR, which are in correlation with the increased immunogenicity of cells in low glucose environment (FIG. 11B).

Increased SAS and Cell Surface Antigen in Senescent TUBO Cells in Low Glucose Media when Treated with Veliparib and IR.

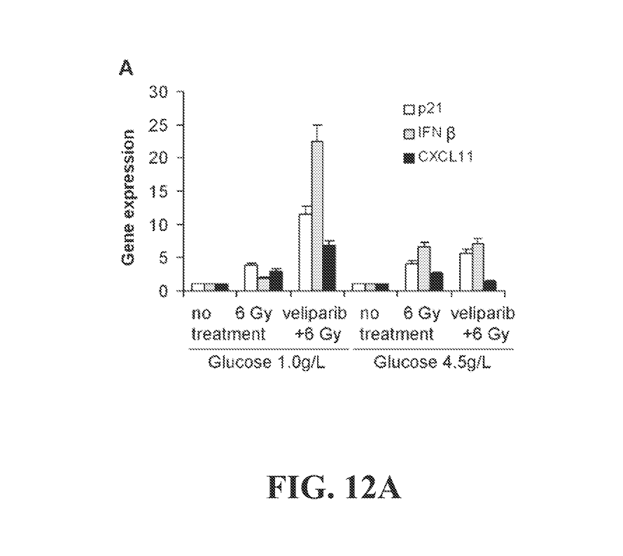

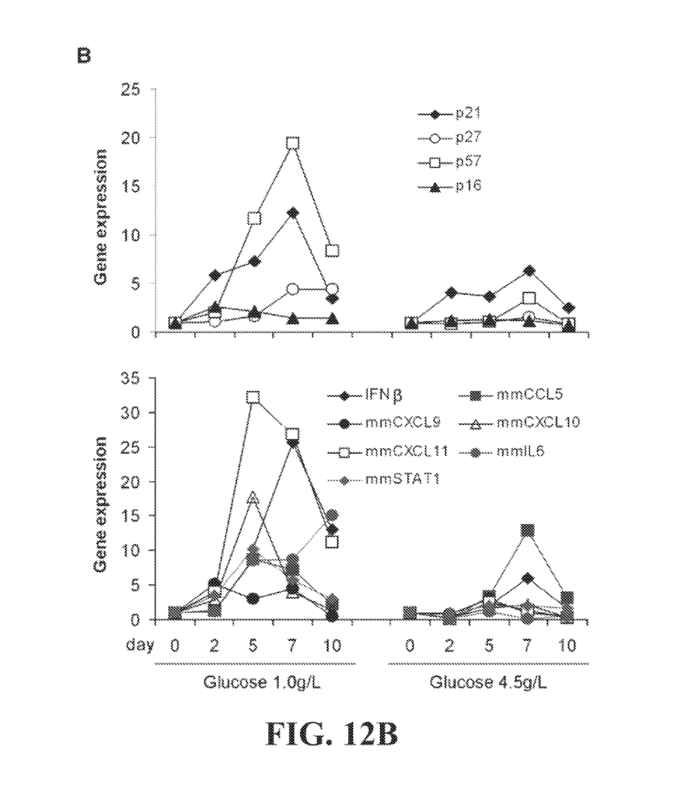

RT-qPCR show that glucose/energy restriction induced early, enhanced and persistent transcription of senescent marker p21CIP1, differentiation marker p57KIP2 and senescence-associated secretory phenotype (SASP) IFNβ, CXCL 10 and CXCL11 (FIG. 12A,B). Treatment of TUBO cells cultured in low glucose medium and treated with veliparib+IR expressed IFNβ and CXCL11 started at day 5 through day 10, while TUBO cells cultured in high glucose media only showed low level IFNβ and CCL5 at day 7 (FIG. 12B). When extracellular ATP release was analyzed, higher ATP was detected in veliparib+IR treated cells when cultured in low glucose media in higher density.

Senescent TUBO Cells Synergize with CpG and IR to Prevent Tumor Regrowth Post IR.

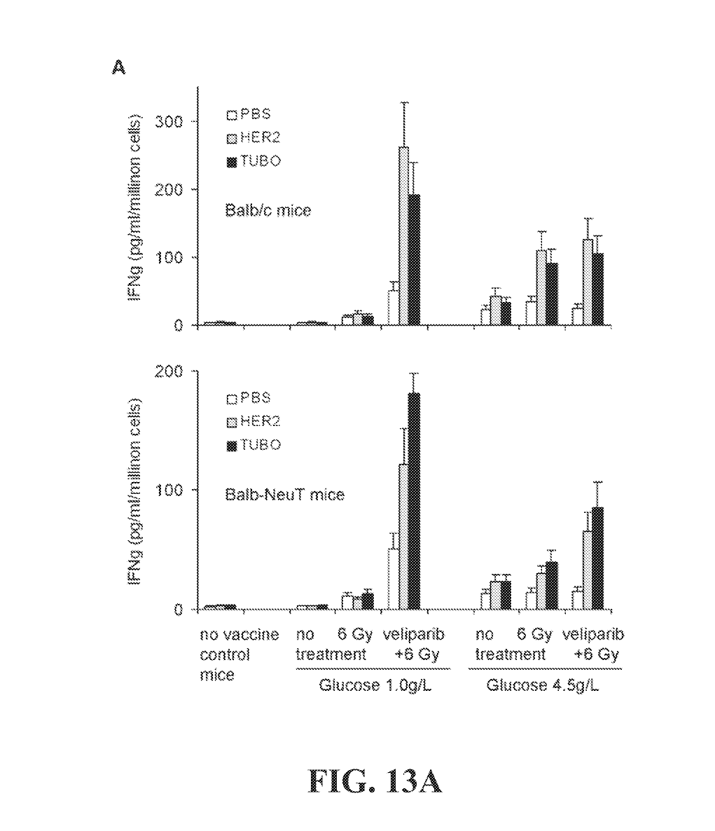

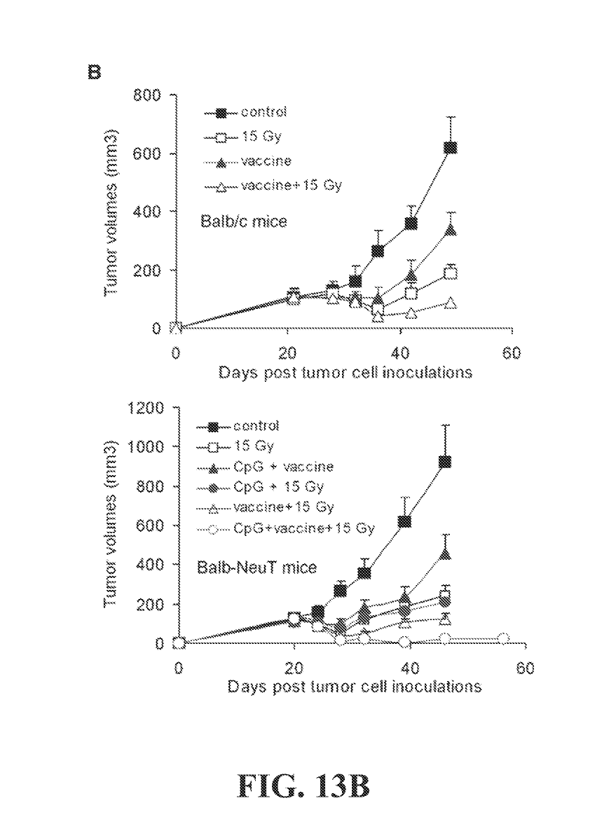

Veliparib+IR-treated TUBO cells growing in low glucose medium induce higher Her2- and tumor-specific IFNγ producing T cells in both non-tolerant Balb/c and tolerant Balb-NeuT mice (FIG. 13A). Inoculation of senescent TUBO cells induced in low glucose medium on left leg also delayed regrowth of tumors on the right leg after IR, suggesting systemic immune activation (FIG. 13B). When this TUBO cell vaccine was combined with TLR9 agonist CpG, which has been increasingly applied in preclinical and clinical studies as a therapeutic agent to enhance tumor immunity, tumor regrowth was greatly delayed post IR when compared to cell vaccine+15 Gy in Balb-NeuT mice (FIG. 13B).

Synergy of Local IR with CpG Based Senescent TUBO Cells Vaccine in a Spontaneous Tolerant Balb/NeuT Model of Breast Cancer.

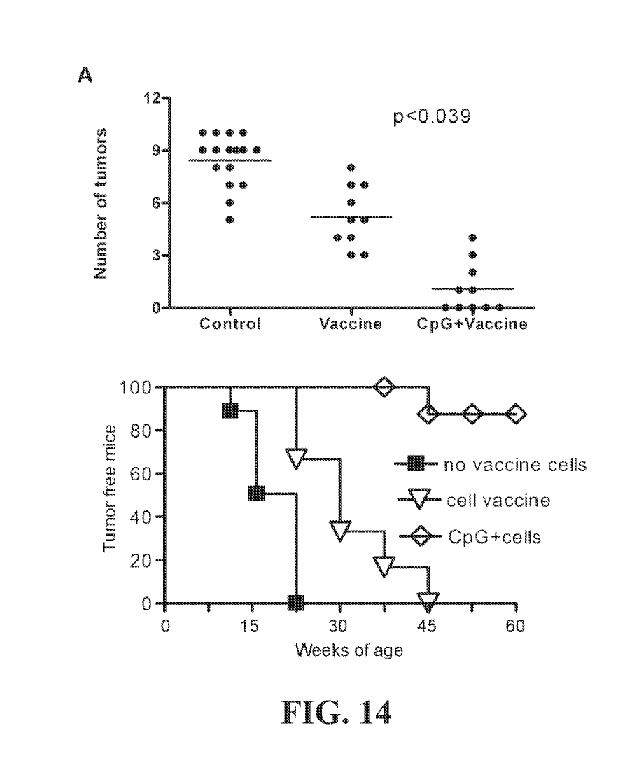

To test if the senescent TUBO cells generated immune response sufficient to prevent mammary carcinogenesis, we inoculated senescent TUBO cells generated in low glucose media treated with veliparib+IR, with or without the adjuvant CpG, in the right leg of 6 week old Balb-NeuT mice, once a week for 3 weeks, after 1 week of rest this course was repeated for another 3 cycles. All mammary glands were inspected, tumor incidence, number of tumors in each mice and mean tumor volumes were measured. All control mice developed their first mammary tumor within 22 wk and tumors in all 10 mammary glands within 30 weeks (FIG. 14A). When mice treated with senescent TUBO cells alone, tumor number and size were significantly decreased, however, when mice were treated with senescent TUBO cells plus CpG, 80% of mice were completely tumor free at 52 weeks (end of the study) and their lifetime was more than doubled (FIG. 14A).

To test if senescent TUBO cell vaccines may increase the effectiveness of anti-tumor effects of local IR in Balb-NeuT spontaneous tumors, we inoculated senescent TUBO cells generated in low glucose media treated with veliparib+IR, in the right leg of 5 month old Balb-NeuT mice which had already developed multiple spontaneous tumors, weekly for 3 weeks. On the 2nd week, the biggest tumor on a single side of the mammary glands received 15 Gy, all tumors in all 10 mammary glands were measured and compared between the irradiated one and non-irradiated ones. Vaccination of senescent TUBO cells delayed irradiated tumor regrowth post local IR, while unirradiated tumors showed short term growth control. This abscopal effect indicated the systemic immune activation. Tumor samples were collected and TILs in the irradiated tumor and unirradiated tumors were analyzed by FACS. The frequency of CD8⁺, CD4⁺CD25⁺FoxP3⁺ Treg and CD11b⁺Gr1⁺ MDSC in CD45⁺ TILs were calculated and the ratios of CD8⁺ T-cell/Treg, CD8⁺ T-cell/MDSC were calculated.

TABLE 3
Senescence inducing compounds and conditions

| | | Cell | | Cell |
|---|---|---|---|---|
| Compound | Condition 1 | line 1 | Condition 2 | Line 2 |
| Trazodone | 10 μM + 7 Gy | B16 | 10 μM + 6 Gy | MCF7 |
| Ketotifen | 25 μM + 7 Gy | B16 | 5 μM + 5 Gy | MCF7 |
| Cephalexin | 50 μM + 7 Gy | B16 | 10 μM + 6 Gy | MCF7 |
| Nisoldipine | 2.5 μM + 7 Gy | B16 | 10 μM + 6 Gy | MCF7 |
| CGS15943 | 25 μM + 7 Gy | B16 | 0.05 μM + 5 Gy | MCF7 |
| Clotrimazole | 2.5 μM + 7 Gy | B16 | not tested | not tested |
| 5-Nonyl- | 5 μM + 7 Gy | B16 | not tested | not tested |
| tryptamine | | | | |
| Doxepin | 2.5 μM + 7 Gy | B16 | 2.5 μM + 5 Gy | MCF7 |
| Pergolide | 10 μM + 7 Gy | B16 | not tested | not tested |
| Paroxetine | 25 μM + 7 Gy | B16 | not tested | not tested |
| Resveratrol | 25 μM + 7 Gy | B16 | not tested | not tested |
| Quercetin | 25 μM + 7 Gy | B16 | 2.5 μM + 5 Gy | MCF7 |
| Honokiol | 5 μM + 7 Gy | B16 | not tested | not tested |
| 7-nitro- | 50 μM + 7 Gy | B16 | not tested | not tested |
| indazole | | | | |
| Megestrol | 25 μM + 7 Gy | B16 | not tested | not tested |
| Fluvoxamine | 10 μM + 7 Gy | B16 | not tested | not tested |
| Etoposide | 1.25 μM + 7 Gy | B16 | not tested | not tested |
| Veliparib | 25 μM + 7 Gy | B16 | not tested | not tested |
| Rucaparib | 25 μM + 7 Gy | B16 | not tested | not tested |
| Olaparib | 10 μM + 7 Gy | B16 | not tested | not tested |
| Campto- | 1.25 μM + 7 Gy | B16 | not tested | not tested |
| thecin | | | | |
| Terbinafine | 2.5 μM + 5 Gy | MCF7 | not tested | not tested |
| Cefaclor | 25 μM + 6 Gy | MCF7 | not tested | not tested |
| Rolipram | 10 μM + 6 Gy | MCF7 | not tested | not tested |
| Pitavastatin | 10 μM + 6 Gy | MCF7 | not tested | not tested |

### Example 3

### Coculturing of DCs with Senescent Cells—Immunostimulation Assay

The bone marrow (BM) cells were isolated and propagated for 5 days as was previously described (Lutz 1999). Briefly 2×10⁶ of collected BM cells were resuspended in 10 ml of complete medium (CM) (RPMI, 10% FBS, pen/strep, HEPES) +20 ng/ml mouse granulocyte-macrophage colony-stimulating factor (GM-CSF). BM were transferred to uncoated plastic Petri dish. On a third day 10 ml of fresh CM+20 ng/ml GM-CSF were added. Immature dendritic cells (DC) were harvested on day 5, were suspended at 5×106 cells/ml in ice-cold freezing medium (CM+10 ng/ml GM-CSF+10% DMSO) and frozen for future experiments. Freezing and thawing of BMDCs was shown to not affect their properties (Sai 2002).

Assayed cells (TRAMP-C2) were plated 2.5×10⁵ p100 plates and next day irradiated (6Gy) and 25 μM veliparib to obtained senescent phenotype. For TRAMP-C2 the coculture was started 6 days after cell irradiation and veliparib treatment. DCs were thawed and plated in CM+10 ng/ml GM-CSF 24 hours before they were cocultured with senescent TRAMP-C2 cells. In a day when co-culture started DCs were detached with trypsin and washed twice with phosphate buffered saline (PBS) to remove GM-CSF. Senescent TRAMP-C2 cells culture was washed with PBS and medium also changed to remove veliparib. Washed DCs and senescent cells were cocultured in fresh medium for 2-3 days.

After coculture medium was collected to harvest non-adherent cells fraction. Adherent cells were detached with trypsin, collected and combined with medium containing non adherent cells, spun down and washed using FACS Buffer (PBS without Ca²⁺ & Mg²⁺, 2 mM EDTA, 2% FBS). Samples were vortexed to break up the cell pellet. Each sample was treated for 10 minutes at room temperature with 50 μL of culture supernatant from 2.4G2 hybridoma to block non-specific Fc receptor binding. Directly after blocking cells were stained for 45 min in 4° C. by adding 100 ul of FACS Buffer containing 0.5 μg of each of PerCP/Cy5.5 anti-mouse CD45 Antibody (Biolegend clone 30-F11) and APC anti-mouse CD11c Antibody (Biolegend clone N418).

After staining cells were washed twice by adding at least 10× volume of FACS buffer and spun down to remove not bound antibody. After the last wash step cell pellets were resuspended in 250 uL of FACS buffer and processed for acquisition with flow cytometry.

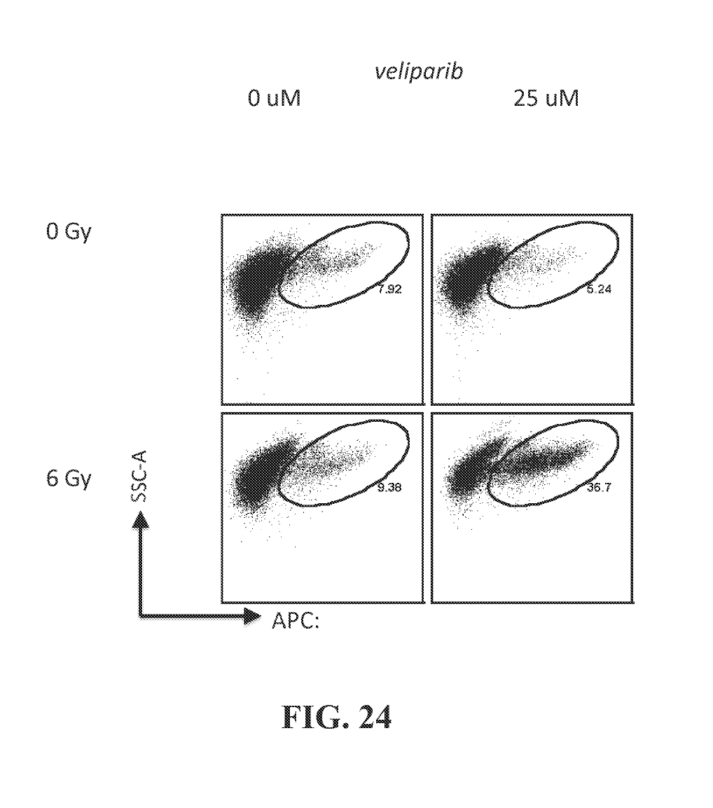

As CD45 is marker of immune cells, only CD45 positive cells were analyzed and TRAMP-C2 cells could be excluded from analysis as CD45 negative. The presented result (FIG. 24) is showing highly enriched population of CD11c positive cells in a sample cocultured with senescent TRAMP-C2 cells obtained with IR (6Gy)+25 μM veliparib treatment (36.7%) comparing to other three conditions (7.9%−0 Gy+O μM veliparib, 5.2%−0 Gy+25 μM veliparib, 9.38%−6Gy+0 μM veliparib). CD11c is a marker of differentiated DC and its higher content can be attributed to an immunostimulatory effect of senescent cells obtained with IR+veliparib treatment.

### Example 4

### Induced Senescence Experimental Data

Inhibition of poly(ADP-ribose) polymerase (PARP) combined with ionizing radiation (IR) delays tumor growth via inducing accelerated senescence of the tumor cells. 5×10⁵ B16SIY murine melanoma tumor cells (B16) derived from C57BL/6 mice were inoculated subcutaneously, and after twenty-one days, the established tumors were treated with the PARP inhibitor veliparib (ABT-888, Abbott) twice daily starting 1 day before, and then daily after irradiation with IR at a dose of 6 Gray (Gy) or 12 Gy. Veliparib+IR treated tumors showed significant growth delay when compared to those treated with 6 Gy or 12 Gy IR alone, p=0.033, p=0.004. n=5-25/group FIG. 1a). Tumors treated as above were collected at 7 days following IR, either fixed/embedded for H/E staining (upper four images) or snap frozen for senescence-associated betagalactosidase (SA-β-Gal) staining (lower four images) (FIG. 1b). Scale bars, 50 μm. B16 cells were treated with veliparib+12 Gy in vitro and incubated 7 days, and then subjected to sorting via flow cytometry, based on separating populations with distinct forward scatter (size, FSC) and side scatter (granularity, SSC). When mice were injected with large (high FSC, high SSC) senescent cells in comparison to the small (low FSC, low SSC) non-senescent, proliferative cells, the large senescent cells (SC) failed to form tumors, while small non-senescent cells (NC) formed tumors readily (FIG. 1c). Coinjection of increasing proportions of senescent cells increasingly inhibited the growth of untreated cells. n=5-10/group.

PARP inhibition modifies immuno-regulatory cytokine components in irradiated B16 tumor cells. Correlation of expression of interferons, chemokines and other immune cell to cell signaling genes with senescent cell cycle arrest associated genes in tumor samples collected from experimental mice analyzed by RT-PCR and normalized with GAPDH. (FIG. 2a) Immunohistochemistry showing IFNβ, CXCL9, CXCL10 and CCL2 staining in large senescent tumor cells present in tumors treated with veliparib+IR. Data are representative of 5 experiments. (FIG. 2b) Scale bars, 50 μm.

CD8⁺ cells inhibit the growth of bystander non-senescent cells. CD8⁺ cells contribute to irradiation effect and tumor growth delay following veliparib+IR. Mice bearing established tumors were treated with veliparib and 12 Gy and with reagents to deplete CD4⁺ T cell, CD8⁺ T cell, NK or macrophage cells. Depletion of CD8⁺ T cells abrogated the tumor growth delay following veliparib+12 Gy, p=0.003. Depletion of NK cells partially reduced the anti-tumor effect of veliparib+12 Gy, p=0.009. n=5-15/group. (FIG. 3a) CD8⁺ cells contribute to IR effect and tumor growth delay post veliparib+IR treatments. n=6-15/group. (FIG. 3b) CD8⁺ T cells maintain the tumor remission following veliparib+IR treatment, as illustrated by the decreased SA-βGal staining and increasing cellularity in CD8⁺ T cell depleted tumors. (FIG. 3c)

Senescent B16 tumor cells enhanced murine bone marrow-derived dendritic cell precursor (BMDC) proliferation, maturation and function to stimulate Th1 response. Coculture with veliparib+IR induced senescent B16 tumor cells promoted BMDC proliferation and maturation, demonstrated by the increased expression of MHC-II and CD86 on CD11c⁺ cells. More larger cells were expanded from smaller immature bone marrow cells which gave rise to CD11c⁺ DC. Data are representative of 4 experiments. (FIG. 4a) BMDC cultured with veliparib+IR induced senescent cells stimulated CD8⁺ cell proliferation as detected by CFSE dilution assay and increased IFNγ production. Data are representative of 3 experiments. (FIG. 4b) Veliparib+IR induced senescent B16 cell elicited an antigen specific antitumor response in draining lymph node (DLN) cells as analyzed by ELISA of IFNγ production after exposure to melanoma antigen gp100. (FIG. 4c) Results are means of duplicate culture with DLN cells collected from 3 individual mice.

PARP inhibition enhanced vaccine potency of irradiated tumor cells. Vaccine effect of B16 cells treated with 6 or 12 Gy alone, veliparib alone or veliparib+6 or 12 Gy compared. Treated B16 cells were injected subcutaneously on the right leg of syngeneic C57BL/6 mice and 7 days later untreated B16 tumor cells were injected in the left leg and tumor formation was followed. Like untreated B16 tumor cells, B16 cells treated with veliparib alone displayed no vaccine effect. While injection of B16 cells treated with 6 or 12 Gy blocked tumor formation in a majority of mice, the veliparib+IR treated B16 cells displayed the strongest vaccine effect. (FIG. 5a) When cells treated with veliparib+IR were subjected to sorting via flow cytometry, based on populations with distinct forward scatter (size, FSC) and side scatter (granularity, SSC), the vaccine effect was specific to the large (high FSC, high SSC) senescent cells and absent from the small (low FSC, low SSC) proliferative cells. (FIG. 5b) Veliparib+IR induced senescent p1048 murine pancreatic tumor cell elicited a more robust vaccine effect compared to p1048 tumor cells IR alone or untreated. (FIG. 5c) Veliparib+IR treated non-senescent TUBO murine mammary tumor cells failed to prevent tumor formation after injection of untreated TUBO cells. (FIG. 5d)

Senescent tumor cells delay the outgrowth of transplanted tumors and potentiate the effects of irradiation, by delaying tumor relapse after IR. 5×10⁵ B16 tumor cells were inoculated subcutaneously on the right leg of syngeneic C57BL/6 mice. After 7 days, the emerging tumors were treated with injection of sorted large senescent tumor cells on the left leg. Significant growth delay was observed when compared to control (p=0.038). Some tumors were treated with 20 Gy, the addition of senescent tumor cells in a remote site delayed tumor growth following IR (p=0.003, n=5/group). (FIG. 6a) The size of tumors surgically removed from different treatment groups can be visualized. (FIG. 6b) FACS analysis of tumor infiltrating CD8⁺ cells reveals increased proportion of IFNγ positive cells when tumors were treated with senescent cell vaccine or IR, and a compound effect when treated with senescent cell vaccine and then IR. (FIG. 6c)

Identification of human cells induced to perform accelerated senescence via detection of senescence associated beta-galactosidase (SA-βGal) by DDAO-G red fluorescent substrate. Flow cytometry of viable cells comparing SA-βGal (B-Gal) vs. side scatter (SSC-A), with senescent gate shown (1.6%). (FIG. 7a) Untreated cells; senescent gated cells (grey) overlaid with total cell population (black) showing forward scatter (size, FSC) vs. side scatter (granularity, SSC) distribution. (FIG. 7b) Viable veliparib+IR treated cells; B-Gal vs. SSC, with senescent gate shown (20%). (FIG. 7c) Veliparib+IR cells; senescent gated cells (grey) overlaid with total cell population (black), FSC vs. SSC distribution. Within the region shown by the black rectangle, 41% of cells are B-Gal^{high} and 59% are B-Gal- or B-Gal^{low}. (FIG. 7d)

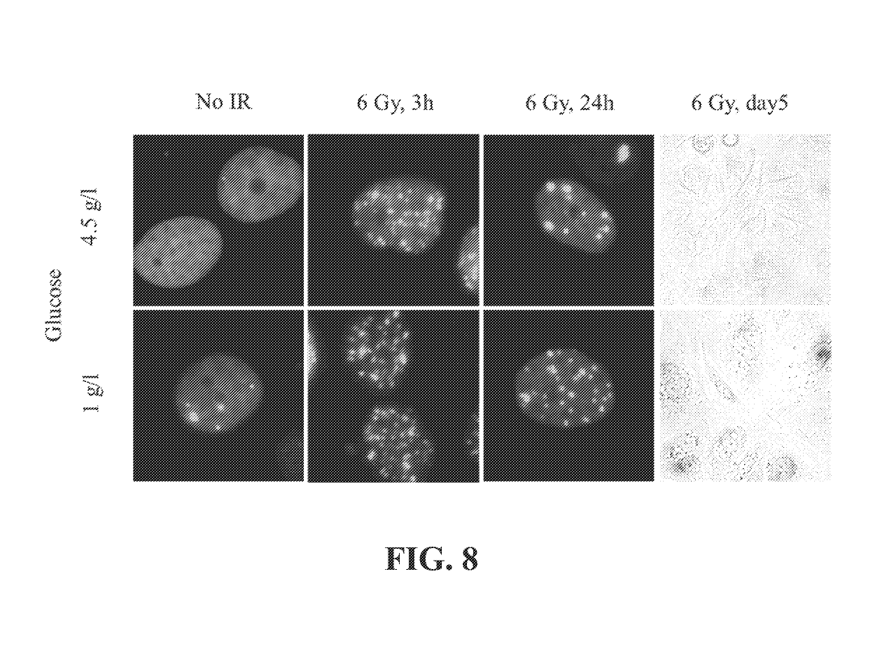

Glucose limitation affects IR-induced foci (IRIF) persistence and senescence in MCF7 cells expressing a GFP fusion to the 53BP1 IRIF binding domain as a reporter (MCF7^{Tet-On} GFP-IBD). Using GFP fluorescence to detect IRIF, cells displayed IRIF at 3 hours after 6 Gy irradiation that resolved more rapidly by 24 hours in cells growing in high glucose (4.5 g/l) media than in low glucose (1 g/l) media. Glucose limitation significantly increased IRIF persistence at 24 hours, based on measuring number of IRIF per cell. Mean IRIF per cell±SEM at 24 h were 8±0.3 for high glucose media and 17±0.9 for low glucose media, P value<0.0001. As shown in left-most images, irradiated cells growing in low glucose media develop senescent morphology and increased SA-βGal activity. (FIG. 8)

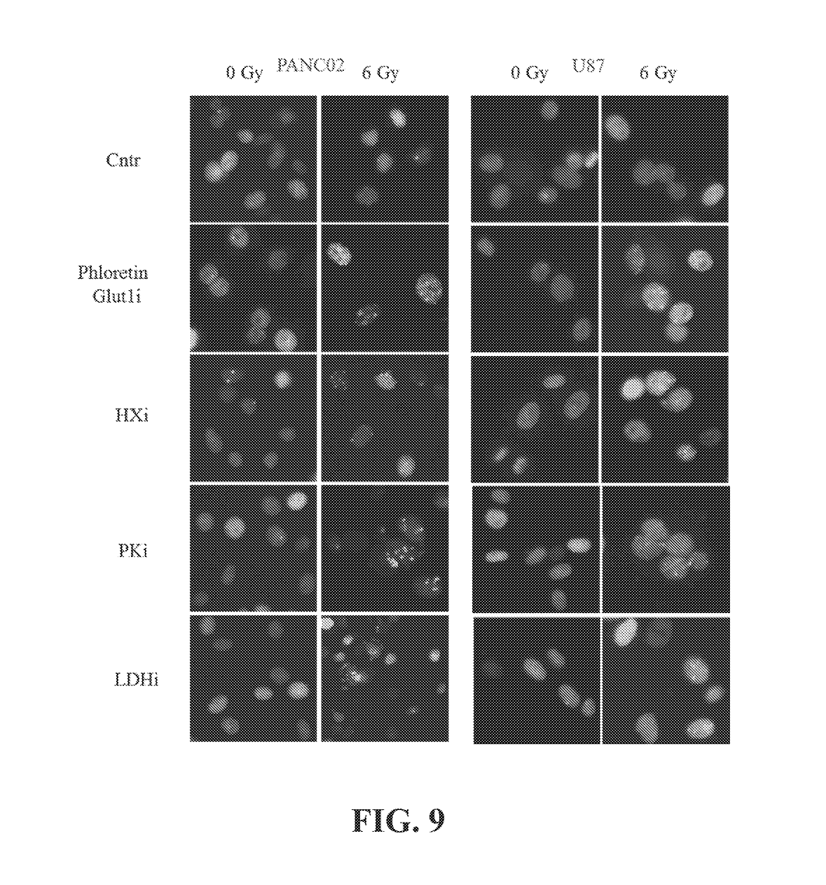

Glycolysis inhibitors overcame the intrinsic radioresistance and induced IRIF persistence in radiation resistant PANC02 mouse pancreatic and U87 human glioma cell lines. PANC02^{Tet-On} GFP-IBD and U87^{Tet-On} GFP-IBD cells expressing the GFP-53BP1 IRIF reporter show pan-nuclear fluorescence before IR treatment and resolve most of the IRIF at 24 h after 6 Gy irradiation. Treating the cells with small molecule glycolysis inhibitors targeting glucose transport (Glut1i), hexokinase (HXi), pyruvate kinase (PKi), and lactate dehydrogenase (LDHi) markedly increased IRIF persistence at 24 hours in both IR resistant cell lines. (FIG. 9)

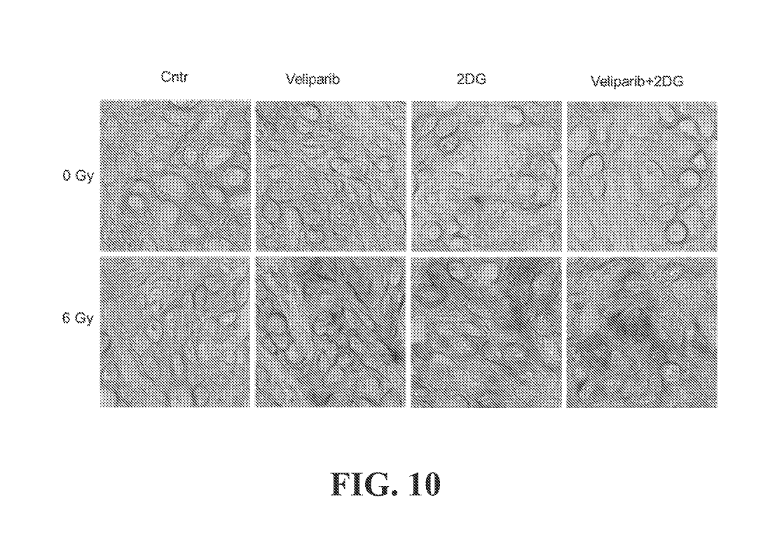

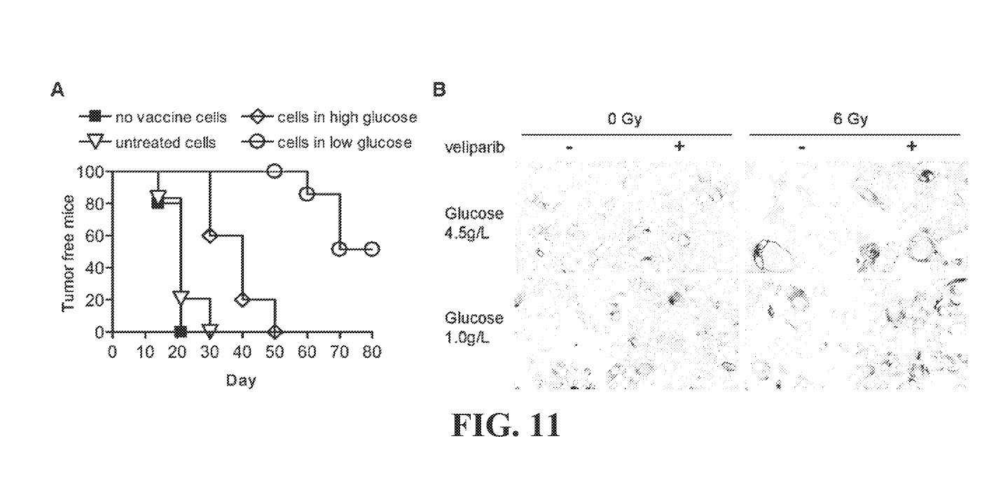

Glycolysis inhibitor 2-deoxy-D-glucose (2DG) combined with irradiation increases cancer cell senescence in vivo in IR-resistant tumor xenografts. In tumors exposed to irradiation alone we did not observe any SA-βGal positive cells. Irradiation combined with glycolysis inhibitor 2DG induced numerous cells that stained positive for SA-βGal, even more then irradiation combined with PARP inhibitor veliparib (positive control). The strongest induction of SA-βGal was observed in irradiated tumors treated with 2DG and veliparib. These data indicate that glycolysis inhibitors may cooperate with PARP inhibitors to promote accelerated senescence in IR-resistant tumors. (FIG. 10)

TUBO murine mammary tumor cells propagated in 1 g/l glucose cell culture media and treated with veliparib+IR prevented tumor growth in mice. (FIG. 11a) TUBO cells growing in 1 g/l glucose media showed enhanced SA-βGal staining when treated with veliparib+IR over cells grown at 4.5 g/l glucose. (FIG. 11b)

Glucose restriction induced an altered senescence associated secretory phenotype pattern (SASP) and cell surface antigen expression in senescent TUBO cells induced in low (1 g/l) glucose media. TUBO cells cultured in low or high glucose media were treated with veliparib+6 Gy or 6 Gy alone. At day 7 tumor cells were analyzed for senescent marker p21 and cytokine/chemokine expression by qRT-PCR. Relative gene expression was compared. (FIG. 12a) Kinetics of gene expression of TUBO cells treated with veliparib+6 Gy which were cultured in low or high glucose media. (FIG. 12b)

FIG. 13. Irradiated senescent TUBO cell vaccine synergized with synthetic adjuvant CpG and IR to prevent tumor growth post IR in syngeneic Balb/c and autochthonous tumor-forming, tolerized Balb-NeuT mice. TUBO cells cultured in low or high glucose media were treated with veliparib+6 Gy or 6 Gy alone and inoculated subcutaneously on the leg. Cells from draining lymph nodes (DNLs) were isolated and cultured with HER2 peptide or TUBO lysate for 5 days. Culture supernatants were collected and IFNγ secretion was tested using ELISA. (FIG. 13a) TUBO tumors were established in syngeneic mice on the right leg. Senescent TUBO cells were obtained by treatment cells with veliparib+6 Gy in low glucose media. At day 21 and 28 after tumor cell inoculations, 5×10⁵ senescent cells were inoculated in the left leg as vaccine. At day 28, tumors on the right leg also received 15 Gy IR. Tumors were measured and calculated as tumor volume (n=5). Arrows indicated times when vaccine cells and/or IR were given. (FIG. 13b)

Irradiated senescent TUBO cell vaccine prevents tumor growth in Balb/NeuT mice. Vaccination of young Balb-NeuT mice with senescent TUBO cells propagated in low glucose media and treated with veliparib+IR in mice reduced the number of tumors developed. (FIG. 14a) Combination with CpG further enhanced the vaccine effect in this model. Combination of vaccine cells+CpG with local IR enhanced the tumor growth delay. Ratios of CD8⁺ cytotoxic T cells to CD4⁺CD25⁺FoxP3⁺ regulatory T cells or CD11b⁺Gr1⁺ myeloid derived suppressor cells in CD45⁺ tumor infiltrating lymphocytes were shown. Values shown are sums of individually analyzed mice.

Enhanced ionizing radiation induced foci (IRIF) formation as detected by immunofluorescence detection of phosphorylated H2AX (γH2AX) and of localization of 53BP1 protein and detection of accelerated senescence by senescence associated beta-galactosidase (SA-βGal) assay in B16SIY murine melanoma cells treated by veliparib and/or 6 Gy ionizing radiation (FIG. 15).

Flow cytometry based sorting of large senescent cells versus small non-senescent cells. B16 cells were treated with veliparib+6 Gy in vitro for 5 days and then subjected to sorting via flow cytometry, based on separating populations with distinct forward scatter (size, FSC) and side scatter (granularity, SSC). Sorted cell were reanalyzed by flow cytometry for their purity (FIG. 16).

Veliparib modifies the SASP in irradiated B16 tumor cells. Kinetics of expression of cell to cell immune signaling mediators IFNβ, CCL5, and CXCL11 correlated with induction of p21 as an indication of senescence development in B16 tumor cells treated with veliparib+IR. (FIG. 17a) Induced expression of IFNβ and chemokine genes in B16 tumor cells induced by veliparib+IR treatment in vitro. (FIG. 17b), (FIG. 17c) Veliparib accelerated cellular senescence in irradiated p1048 cells visualized by SA-βGal staining. (FIG. 17d) Higher IFNβ and chemokine gene expression in p1048 cells at 7 days after treatment with veliparib+IR. (FIG. 17e)

Flow cytometry analysis of tumor infiltrating lymphocytes (TILs) from B16 tumors treated with veliparib with or without irradiation. Greater numbers of IFNγ expressing CD8⁺ and NK cells were detected in veliparib+12 Gy treated tumors, suggesting an anti-tumor immune response (FIG. 18)

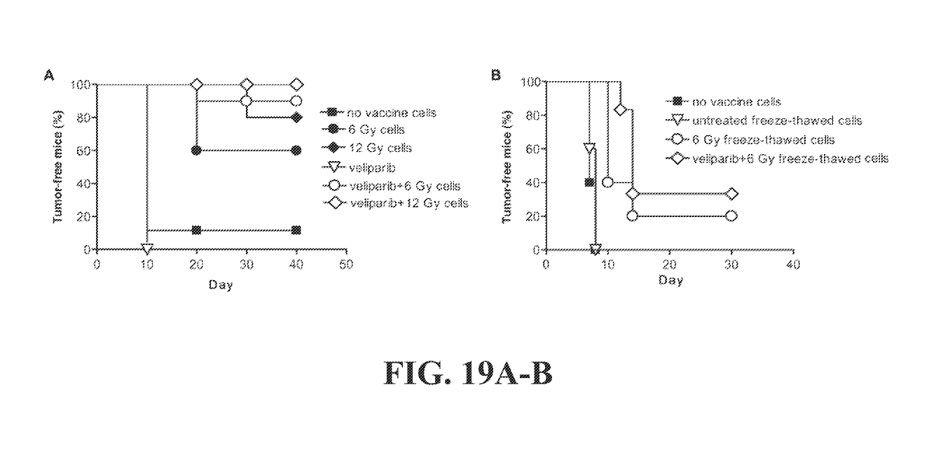

Veliparib+IR treated senescent B16 tumor cell vaccines provide protection against tumor formation after challenge by injection of untreated B16 tumor cells, compared to vaccines prepared from B16 cells that were treated with either veliparib alone, IR alone or untreated. 5 days following vaccination, mice were injected with B16 tumor cells on the left leg. The percentage of tumor-free mice was followed. (FIG. 19a) Freeze thawed tumor cells have also been used in vaccine trials. To investigate the effect of freeze-thawing, untreated B16 cells, B16 cells treated only with IR and cells treated with veliparib+IR as for (FIG. 19a) were transferred between room temperature and liquid nitrogen for 5 cycles and then injected into the right leg. After 7 days, the mice were challenged with untreated B16 cells. Multiple cycles of freeze-thaw treatment markedly decreased the vaccine effect of both the IR and veliparib+IR treated cells. (FIG. 19b)

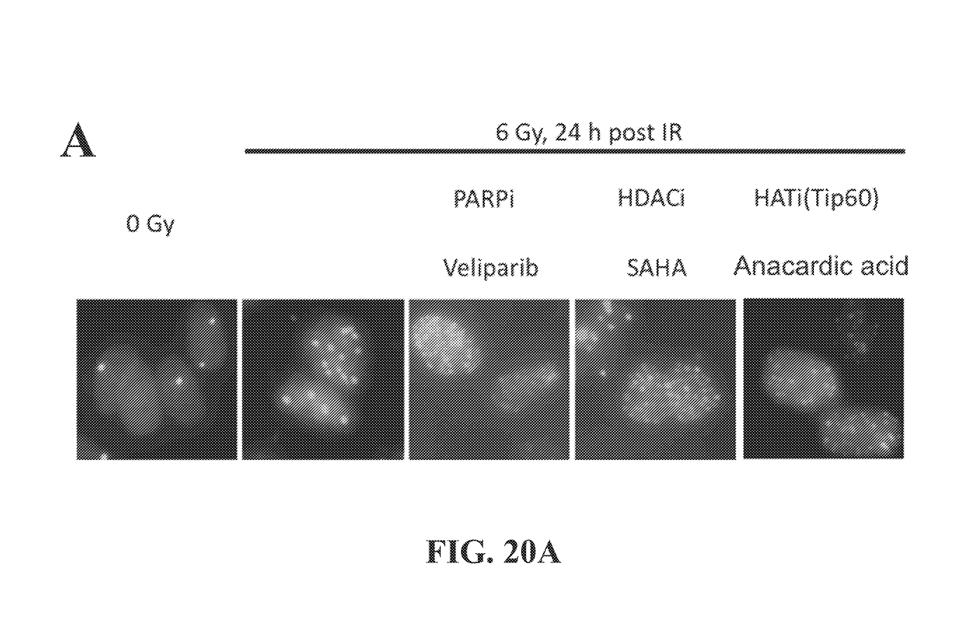

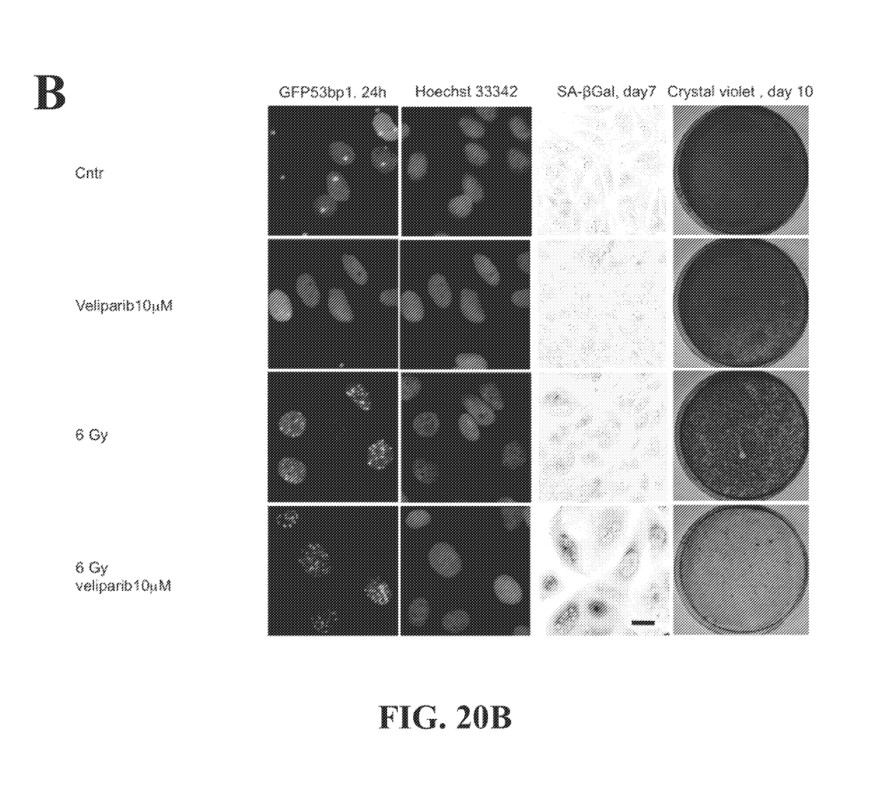

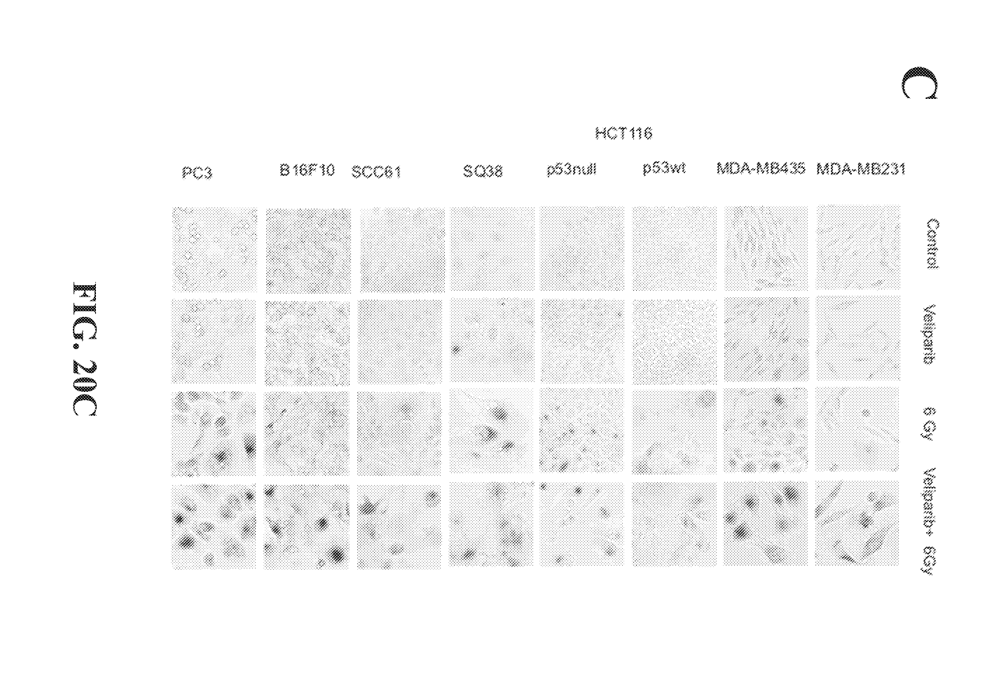

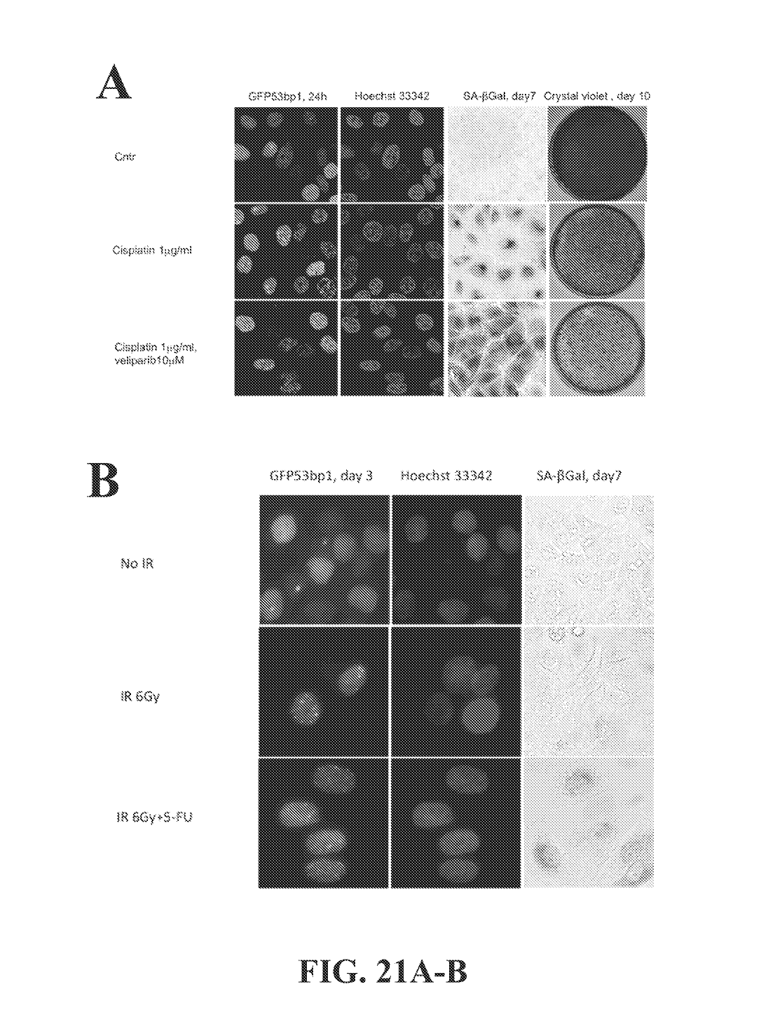

Drugs targeting chromatin modification and DNA repair enhanced radiation induced persistence of GFP-53BP1 foci as a reporter of IRIF in MCF7^{Tet-on} GFP-IBD human breast cancer cell line. PARP inhibitor (PARPi) veliparib, histone deacetylase inhibitor (HDACi) SAHA (vorinostat, suberoylanilide hydroxamic acid), and histone acetyl transferase (Tip60) inhibitor (HATi) anacardic acid enhance radiation induced persistence of GFP-53BP1 foci MCF7 cells. (FIG. 20a) Compared to veliparib or radiation alone, veliparib+6 Gy promotes persistence of GFP-53BP1 foci, induces accelerated senescence and causes growth suppression in MCF7. (FIG. 20b) Veliparib enhances radiation induced senescence in different human cancer cell lines, including breast, prostate, melanoma and head and neck squamous cell cancer cell lines. (FIG. 20c)

Combining chemotherapy agents with veliparib induced accelerated senescence. Cisplatin induced persistence of GFP-53BP1 foci in MCF7^{Tet-on} GFP-IBD cell line, resulting in accelerated senescence and growth suppression. Veliparib enhances this effect. (FIG. 21a) Fluorouracil (5-FU) enhances IRIF persistence and accelerates senescence in MCF7 cell line. (FIG. 21b)

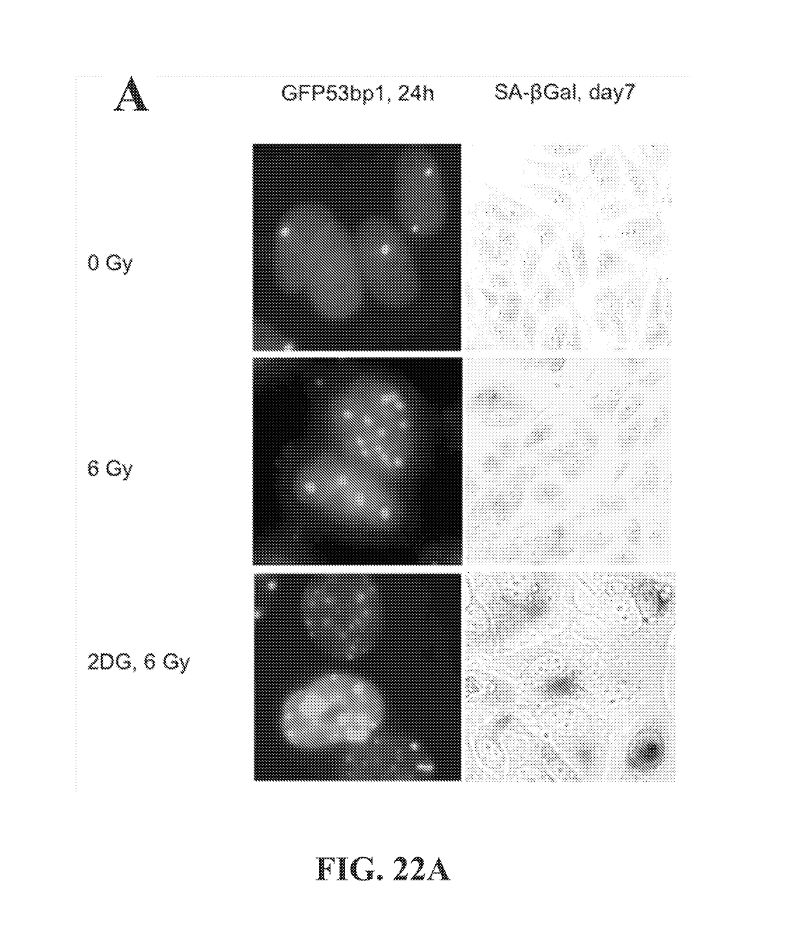

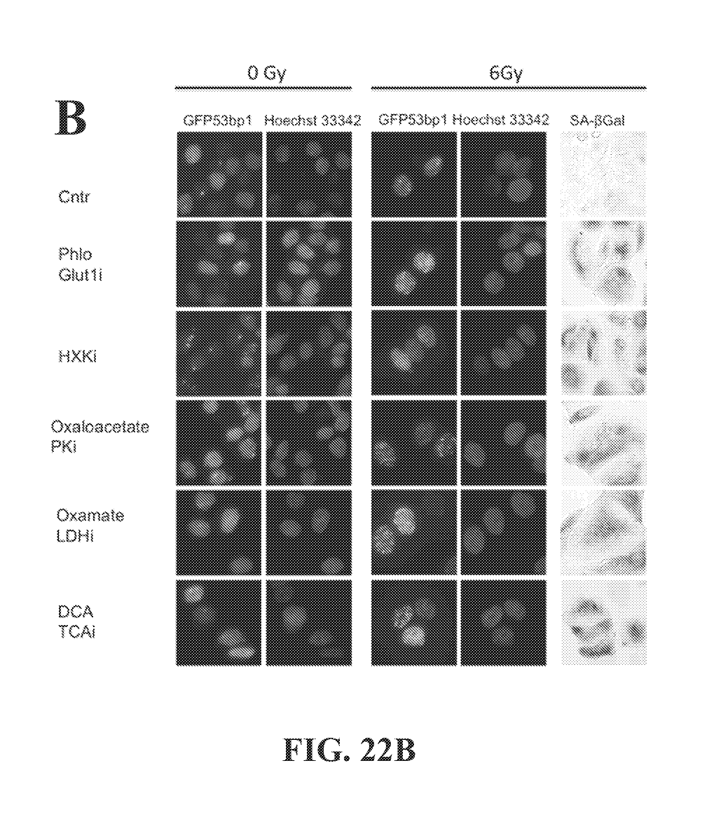

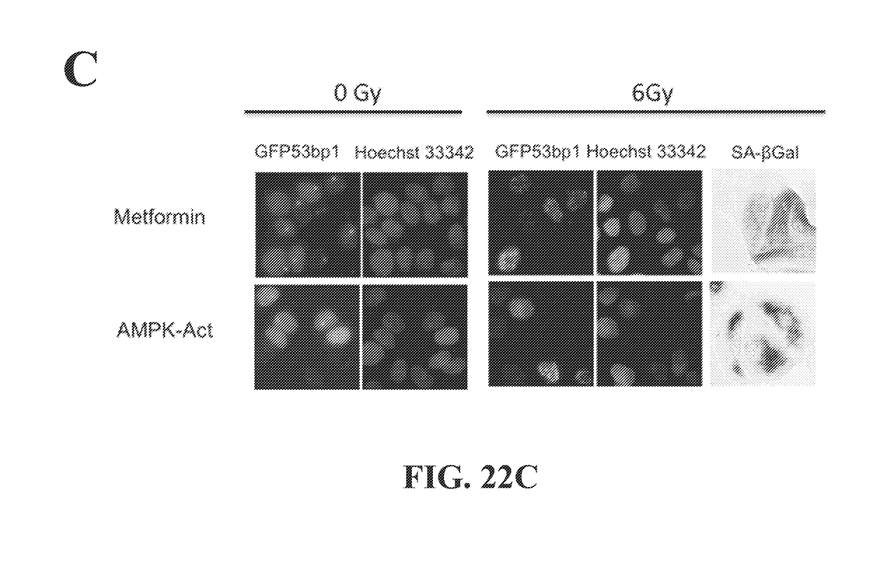

Glucose metabolism inhibitors induced senescence in irradiated tumor cells. 2-deoxyglucose induced persistence of GFP-53BP1 foci following senescence in irradiated MCF7^{Tet-on} GFP-IBD cells. (FIG. 22a) Glycolysis inhibitors including Glut1 inhibitor (Glut1i) phloretin (Phlo), hexokinase inhibitor (HXKi), pyryuvate kinase inhibitor (PKi) oxaloacetate, lactate dehydrogenase inhibitor (LDHi) oxamate and TCA cycle inhibitor (TCAi) dichloroacetic acid (DCA) all induced persistence of GFP-53BP1 foci following irradiation and promoted accelerated senescence in MCF7 cells. (FIG. 22b) Adenosine Monophosphate-Activated Protein Kinase (AMPK) activators metformin and compound C induced persistence of GFP-53BP 1 foci after irradiation and promoted accelerated senescence in MCF7 cells. (FIG. 22c)

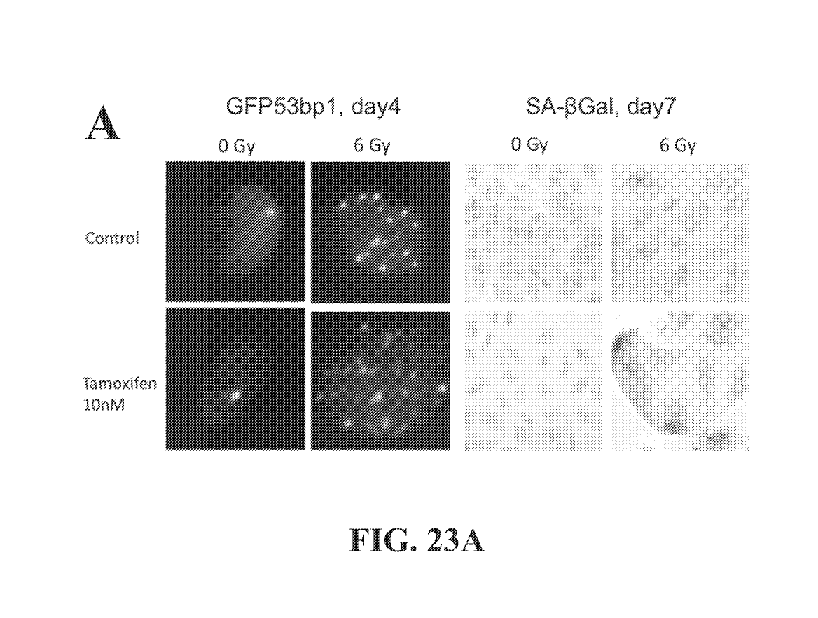

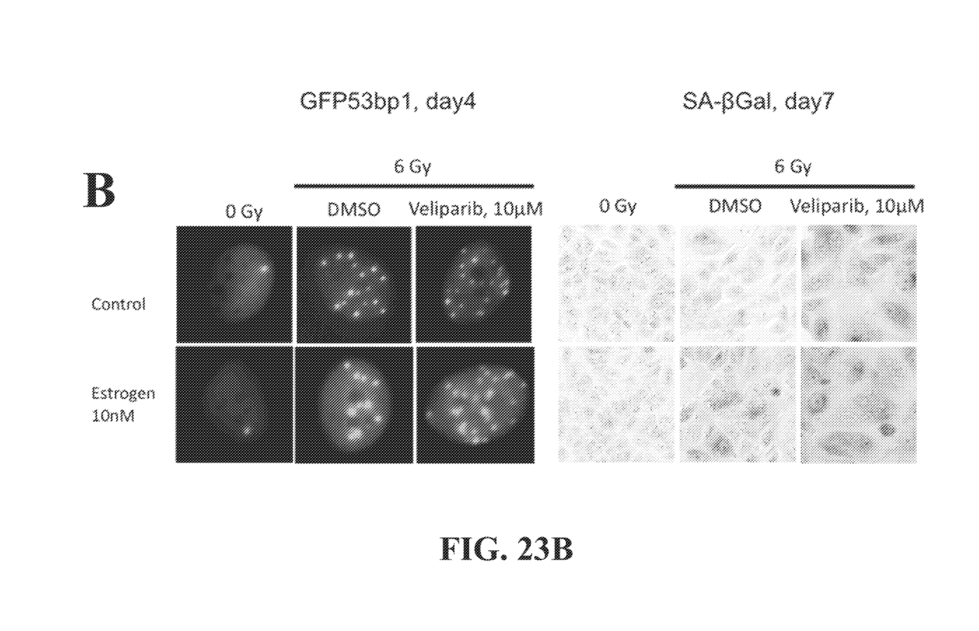

Senescence in hormone dependent tumors. Tamoxifen induced persistence of GFP-53BP1 foci after irradiation and promoted accelerated senescence in MCF7 cell line. (FIG. 23a) Veliparib overcomes the activity of estrogen by promoting persistence of GFP-53BP1 foci and inducing accelerated senescence in irradiated MCF7 cells. (FIG. 23b)

Immunostimolatory effect of senescent TRAMP-C2 cells obtained with combined IR(6Gy)+25 μM veliparib assessed as increased population of Cd11c positive cells—characteristics of differentiated DC. (FIG. 24)

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barreto-Andrade, J. C., Mol Cancer Ther. 10(7): 1185-93, 2011.
Burnette, B. C., et al., Cancer Res. 71(7): 2488-96, 2011.
Campisi, J., Annu Rev Physio. 75: 17.1-17.21, 2013.
Coppe, et al., Annu Rev Pathol. 5: 99-118, 2010.
Dunn, G. P., et al., Nat Rev Immunol. 6(11): 836-48, 2006.
Efimova E V, et al., Cancer Res. 70(15): 6277-82, 2010.
Efimova, E. V., et al., Cancer Res. 70(15): 6277-82, 2010.
El Mjiyad, N., et al., Oncogene. 30(3): 253-64, 2011.
Fowler, J. F., et al., In Stereotactic Body Radiation Therapy. USA, 2005.
Knight, D., et al., Prostate. 69(2): 142-8, 2009.
Kwak, H. et al., Cancer Res. 64(2): 572-580, 2004.
Lee. B. Y., et al., Aging Cell. 5(2): 187-195, 2006.
Lee, Y., et al., Blood. 114(3): 589-95, 2009.
Liu, et al., Am Journ Pathol. 180(2): 599-607, 2012.
Lugade, A. A., et al., J Immunol. 180(5): 3132-9, 2008.
Lutz, M. B., et al., J Immunol Methods. 223(1): 77-92, 1999.
Masuelli, L., et al., Int J Oncol. 30(2): 381-92, 2007.
Meng, Y., et al., Cancer Res. 70(4): 1534-43, 2010.
Meng, Y., et al., Mol Ther. 18(5): 912-20, 2010.
Novakova, Z., et al., Oncogene. 29(2): 273-84, 2010.
Orjalo, A. V., et al., Proc Natl Acad Sci USA. 106(40): 17031-6, 2009.
Penning, T. D., Curr Opin Drug Discov Devel. 13(5): 577-86, 2010.
Rakhra, K., et al., Cancer Cell. 18(5): 485-98, 2010.
Reu, F. J., et al., J Clin Oncol. 24(23): 3771-9, 2006.
Rodier, F., et al., Nat Cell Biol. 11(8): 973-9, 2009.
Sai, T., et al., J Immunol Methods. 264(1-2): 153-62, 2002.
Stangl, S., et al., Proc Natl Acad Sci USA. 108(2): 733-8, 2011.
Tang, X., et al., Cell. 131(1): 93-105, 2007.
Toussaint, O., et al., Exp Gerontol. 35(8): 927-945, 2000.
Xue, W., et al., Nature. 445(7128): 656-60, 2007.
Zhang, B., et al., J Clin Invest. 118: 1398-404, 2008.

1. A method for preparing a pharmaceutical composition of induced senescent cells comprising:
a) exposing cancer cells removed from a patient to an effective amount of radiation and/or at least one senescence inducing agent to induce senescence;
b) purifying or enriching for induced senescent cells; and
c) preparing a pharmaceutical composition of induced senescent cells.

2. The method of claim 1, wherein between about 10⁴ to about 10⁷ cancer cells are exposed to an effective amount of radiation and/or at least one senescence inducing agent.

3. (canceled)

4. The method of claim 1, wherein the cancer cells are exposed to between about 2 and about 20 Gy of radiation.

5. The method of claim 1, wherein the cancer cells are exposed to an effective amount of at least one senescence inducing agent.

6. (canceled)

7. The method of claim 1, wherein the at least one senescence inducing agent is a tumor suppressor inducer, mitotic inhibitor, nucleic acid damaging agent, antitumor antibiotic, topoisomerase inhibitor, hormone inhibitor, growth factor inhibitor, or PARP inhibitor.

8. The method of claim 1, wherein the at least one senescence inducing agent is Trazodone, Ketotifen, Cephalexin, Nisoldipine, CGS15943, Clotrimazole, 5-Nonyltryptamine, Doxepin, Pergolide, Paroxetine, Resveratrol, Quercetin, Honokiol, 7-nitroindazole, Megestrol, Fluvoxamine, Etoposide, Veliparib, Rucaparib, Olaparib, Camptothecin, or Terbinafine.

9. The method of claim 1, wherein the cancer cells are exposed to radiation and at least one senescing inducing agent.

10. (canceled)

11. The method of claim 1, wherein the induced senescent cells are enriched or purified by sorting induced senescent cells from non-induced senescent cells.

12. (canceled)

13. The method of claim 11, wherein the sorting comprises using flow cytometry.

14.-16. (canceled)

17. The method of claim 1, wherein the induced senescent cells are enriched to produce a population of induced senescent cells that are at least about 80% pure.

18. The method of claim 1, further comprising obtaining the cancer cells from the patient.

19. (canceled)

20. The method of claim 1, wherein the induced senescent cells have a least one of the following characteristics compared to cancer cells not exposed to radiation and/or a senescence inducing agent: reduced cell proliferation rate; increased β-galactosidase activity; increased size; reduced expression of p16INK4a; increased expression of p21Cip1p; increased lyosomal mass; nuclear loci of persistent DNA damage response; and, altered expression or secretion of amphiregulin, growth-related oncogene (GRO) γ, interleukin 6 (IL-6), IL-8, VEGF, and/or matrix metalloproteinase.

21. (canceled)

22. A method for treating a patient for cancer comprising administering to the patient induced senescent cells, wherein the induced senescent cells are derived from cancer cells obtained from the patient.

23. The method of claim 22, wherein the induced senescent cells were prepared by exposing cancer cells from the patient to an effective amount of radiation and/or at least one senescence inducing agent.

24.-45. (canceled)

46. A pharmaceutical composition comprising induced senescent cells, wherein the induced senescent cells have a least one of the following characteristics compared to cancer cells not exposed to radiation and/or a senescence inducing agent: reduced cell proliferation rate; increased β-galactosidase activity; increased size; reduced expression of p16INK4a; increased expression of p21Cip1p; increased lyosomal mass; nuclear loci of persistent DNA damage response; and, altered expression or secretion of amphiregulin, growth-related oncogene *GRO) γ, interleukin 6 (IL-6), IL-8, VEGF, and/or matrix metalloproteinase.

47.-50. (canceled)

51. A method for preparing a pharmaceutical composition comprising antigen presenting cells comprising exposing antigen presenting cells to induced senescent cells; and, preparing a pharmaceutical composition comprising exposed antigen presenting cells.

52. The method of claim 51, further comprising preparing induced senescent cells from a patient.

53. The method of claim 52, wherein preparing induced senescent cells comprises
a) exposing cancer cells removed from a patient to an effective amount of radiation and/or at least one senescence inducing agent to induce senescence;
b) purifying or enriching for induced senescent cells; and

54.-113. (canceled)
